(19) United States
(12) Patent Application Publication
(10) Pub. No.: US 2026/0284421 A1
(43) Pub. Date: Sep. 24, 2026

(54) Double Core-Shell Nanodisc Synthesis and Application to Magnetoelectric Bio-Signaling

(71) Applicant: Massachusetts Institute of Technology, Cambridge, MA (US)

(72) Inventors: Polina Olegovna ANIKEEVA, Lexington, MA (US); Ye Ji KIM, Cambridge, MA (US); Florian KOEHLER, Somerville, MA (US)

(73) Assignee: Massachusetts Institute of Technology, Cambridge, MA (US)

(21) Appl. No.: 19/474,027

(22) PCT Filed: Apr. 15, 2024
(86) PCT No.: PCT/US2024/024604

§ 371 Date: Oct. 9, 2025

Related U.S. Application Data
(60) Provisional application No. 63/496,107, filed on Apr. 14, 2023.

(51) Int. Cl. A61N 1/40; A61N 2/00; B82Y 5/00; B82Y 25/00; B82Y 40/00

(52) U.S. Cl. CPC A61N 1/40; A61N 2/006; B82Y 5/00; B82Y 25/00; B82Y 40/00

(57) ABSTRACT

Deep-brain stimulation (DBS) with implanted electrodes revolutionized treatment of movement disorders and empowered neuroscience studies. Identifying less invasive alternatives to DBS may further extend its clinical and research applications. Nanomaterial-mediated transduction of magnetic fields into electric potentials offers an alternative to invasive DBS. Core-double shell g magnetoelectric nanodiscs (MENDs), such as Fe₃O₄—CoFe₂O₄—BaTiO₃, with efficient magnetoelectric coupling are disclosed. The MENDs may exhibit a robust response to magnetic field stimulation in neurons decorated with MENDs at a density of 1 μg/mm² even though individual-particle potentials may be below the neuronal excitation threshold. MENDs injected into the ventral tegmental area of genetically intact mice at concentrations of 1 mg/mL may enable remote control of reward behavior and provide a mechanistic optimization of magnetoelectric neuromodulation.

### CROSS-REFERENCE TO RELATED PATENT APPLICATION(S)

This patent application claims the priority benefit, under 35 U.S.C. 119(e), of U.S. Application No. 63/496,107, filed Apr. 14, 2023, which is incorporated herein by reference in its entirety for all purposes.

### GOVERNMENT SUPPORT

This invention was made with government support under AT011991 awarded by the National Institutes of Health. The government has certain rights in the invention.

### BACKGROUND

The prevalence of psychiatric and neurodegenerative disorders, resulting in more than 8 million deaths per year, has increased the demand for innovative tools to study and modulate the billions of neurons in the human body. The 1.3-liter human brain comprises thousands of genetically and structurally defined subtypes of neurons, communicating with each other through quadrillions of synapses using electrical signals. Artificial stimulations can be used to assist natural neuronal communication for disease therapies, disability accommodation, or neuronal network studies and can be realized through microfabricated neural probes, electrodes, or fibers. To enhance cellular specificity for improved understanding of basic mechanisms and therapeutic outcomes of electrical stimulations, optogenetics has been adopted to probe and perturb the activity of genetically defined neuronal populations. However, wire connectivity and genetic modification to express the stimulation-sensitive factor limit the application of neural modulation techniques in human clinical approaches.

For example, deep brain stimulation (DBS) via surgically implanted electrodes is a powerful therapeutic tool for neurological and psychiatric conditions. However, the invasiveness of DBS often makes it a last-resort therapy and motivates investigation of less-invasive brain stimulation alternatives. Non-invasive neuromodulation approaches, such as transcranial magnetic stimulation, temporal interference electrical stimulation, and focused ultrasound, eliminate the need for implantable hardware but offer limited resolution especially in deeper brain structures.

Methods combining biologically imperceptible optical, magnetic, or acoustic signals with a genetic or synthetic transducer decouple penetration depth from the resolution serving as a middle ground between invasive DBS and non-invasive low-resolution alternatives. Advances in sensitive opsins recently enabled optogenetic neuromodulation with external light sources. However significant scattering at tissue interfaces still precludes the deployment of these tools in the deep brain, especially in larger organisms. For contactless neural stimulation, optical, acoustic, and magnetic stimuli have been developed. Magnetic fields have larger penetration depths (e.g., >cm) than optical and acoustic fields and can securely reach deep brain regions in the human body without attenuation due to the low magnetic susceptibility of body tissue. Weak magnetic fields (MFs) take advantage of the low conductivity and magnetic permeability of biological matter to deliver signals to deep brain structures. MFs have been transduced into mechanical torque, heat, and chemical release enabling modulation of cells expressing mechano-, thermo-, and chemo-receptors, respectively. Although genetic sensitization of identifiable cells to specified stimuli empowers fundamental neuroscience research, the need for transgenes impedes the implementation of these methods in translational studies and in clinic.

To eliminate the need for transgenes and match the effects of clinical DBS, acoustic fields and MFs have been converted directly into electrical signals via synthetic piezoelectric and magnetoelectric (ME) transducers, respectively. Compared to acoustic signals, MFs offer greater penetration depth, and ME transduction at the millimeter scale has been leveraged to power miniature electrical stimulators with the added benefit of multiplexing. To permit ME neuromodulation with transducers that can be delivered via an injection rather than a surgical placement, an MF must be converted into electrical polarization at the nanoscale. However, achieving stimulation effects comparable to clinical DBS with ME nanomaterials is challenging due to their low generated potentials. Magnetic fields can be transduced into electrical signals that actuate neurons in the body with magneto-electric nanoparticles (MENPs) that are suspended in solution and injected into the body. MENPs controlled by magnetic fields enable neural modulation without genetic modification. Moreover, neurons respond to stimulation with MENPs at rates comparable to those for wire-connected electric stimulation.

Unfortunately, it is not clear whether conventional MENPs can modulate neurons effectively. As currently understood, neurons only respond to and transmit electrical signals larger than the action potential, which is in the range of tens of millivolts. Theoretically, a single conventional CoFe₂O₄—BaTiO₃ core-shell MENP with a 200-300 nm diameter can generate a potential of up to about 1 mV under a 13 mT alternating current (AC) magnetic field, which is lower than the action potential and possibly too low to modulate neurons. However, these values are 2-3 orders of magnitude lower than the changes in membrane voltage necessary to trigger action potentials in neurons. Consequently, conventional MENPs need to be employed at high concentrations, resulting in the formation of microscale aggregates with variable properties.

### SUMMARY

Herein, MENPs that can generate higher potentials under a given magnetic field thanks to an inventive core-double shell structure are disclosed. Unlike a conventional MENP, an inventive MENP may include a hexagonal magnetite (Fe₃O₄) nanodisc with a magnetostrictive (e.g., CoFe₂O₄) inner shell and a piezoelectric (e.g., BaTiO₃) outer shell. MENPs with this double core-shell structure may also be called magnetoelectric nanodiscs (MENDs). MENDs may have an enhanced potential and may enable robust, minimally invasive DBS at low concentrations of synthetic materials.

A MEND may have a core-double shell architecture (e.g., Fe₃O₄—CoFe₂O₄—BaTiO₃) and may have layers of magnetostrictive CoFe₂O₄ and piezoelectric BaTiO₃ grown on Fe₃O₄ templates or magnetite nanodiscs (MND). MENDs may exhibit an enhanced ME coupling coefficient as compared to isotropic MENPs, such as isotropic CoFe₂O₄—BaTiO₃ nanoparticles. For example, a MEND may have a ME coupling coefficient of approximately 160 mV/mT-cm whereas an isotropic nanoparticle may have a ME coupling coefficient of approximately 30 mV/mT-cm. When decorated onto neuronal membranes in vitro at densities of approximately 0.5-1 μg/mm², the MENDs may mediate robust and rapid excitation with combined OMF (220 mT) and AMF (10 mT) at frequencies up to 150 Hz. The voltages generated by individual MENDs may be integrated across the periods of the driving AMF and across multiple particles. The change in membrane potential may increase with increasing AMF frequency commensurate with the scaling of the ME coefficient.

Additionally, decreasing the inter-particle spacing on neuronal membranes may reduce the latency for potential threshold crossing and action potential firing. MEND- mediated stimulation to experiments in vivo may achieve remote magnetic control of neuronal activity in the VTA of wild-type mice at particle concentrations as low as approximately 0.5 mg/mL (1.5 μL injections). Robust neural excitation in the VTA mediated by MENDs may enable remote magnetic control of behavior, driving conditioned place preference in genetically intact mice, suggesting MENDS may provide a minimally invasive alternative to electrical DBS. MEND technology may empower applications where implementation of genetic neuromodulation methods or invasive hardware is challenging, such as experiments in translational non-rodent models or in peripheral nervous system targets.

MENDs may have shape anisotropy and interfacial strain in the Fe₃O₄—CoFe₂O₄ magnetostrictive interior, which may yield gains in strain-mediated ME coupling with the BaTiO₃ shell and lead to an induced robust neuronal response to MF stimuli. MENDs can be used for in vitro and in vivo neural modulation in a solution with a MEND concentration of about 1 mg/mL, which is 100 times lower than the concentration for other magnetic-particle-based neural modulation. For example, the theoretical magnetoelectric (ME) coefficient of a CoFe₂O₄—BaTiO₃ MEND is about 10 V cm⁻¹ Oe⁻¹, which suggests that by preventing charge leakage between cores of adjacent nanoparticles with isolating magnetostrictive cores, about 1 mV can be generated from every single particle under 10 mT and 100 Hz of an AC magnetic field. The MENDs disclosed herein may provide a minimally invasive alternative for DBS.

A MEND's magnetite core can have a maximum lateral dimension of about 170 nm to about 550 nm and a maximum thickness of up to 50 nm. The magnetostrictive shell and the piezoelectric shell of a MEND can each have a thickness of about 5 nm. The magnetostrictive shell can include AₓFe₃₋ₓO₄, where A is one of Co, Mn, Ni, Fe, Zn, Mg, or Cu and x may be any value between 0 and 1. The piezoelectric shell can include BaTiO₃. And the anisotropic magnetoelectric nanoparticle can have a magnetoelectric coupling coefficient of about 130-180 mV/mT-cm under a 220 mT, 150 Hz alternating current magnetic field.

An anisotropic magnetoelectric nanoparticle can be made by forming a magnetite nanodisc via reduction of hexagonal nanodisc hematite, forming a magnetostrictive shell on the magnetite nanodisc, and forming a piezoelectric shell on the magnetostrictive shell to yield the anisotropic magnetoelectric nanoparticle. Forming the magnetostrictive shell on the magnetite nanodisc may include growing a plurality of layers of magnetostrictive material on the magnetite nanodisc. If desired, anisotropic magnetoelectric nanoparticles can be dispersed in a biocompatible solvent (e.g., phosphate buffered saline).

In some aspects, the techniques described herein relate to a method of making an anisotropic magnetoelectric nanoparticle the method including forming a magnetite nanodisc via reduction of hexagonal nanodisc hematite, forming a magnetostrictive shell on the magnetite nanodisc, and forming a piezoelectric shell on the magnetostrictive shell to yield the anisotropic magnetoelectric nanoparticle.

In some aspects, the techniques described herein relate to a method wherein the magnetite nanodisc has a maximum lateral dimension of about 170 nm to about 550 nm and a maximum thickness of up to about 50 nm.

In some aspects, the techniques described herein relate to a method wherein forming the magnetite nanodisc via reduction of hexagonal nanodisc hematite includes controlling a percentage of water in the reduction.

In some aspects, the techniques described herein relate to a method wherein the percentage of water is 6-9%.

In some aspects, the techniques described herein relate to a method wherein forming the magnetostrictive shell on the magnetite nanodisc includes growing a plurality of layers of magnetostrictive material on the magnetite nanodisc.

In some aspects, the techniques described herein relate to a method wherein the magnetostrictive shell includes AxFe3−xO4, where A is one of Co, Mn, Ni, Fe, Zn, Mg, or Cu.

In some aspects, the techniques described herein relate to a method wherein forming the magnetostrictive shell on the magnetite nanodisc includes growing magnetostrictive material to a thickness of up to about 5 nm on the magnetite nanodisc.

In some aspects, the techniques described herein relate to a method wherein forming the piezoelectric shell on the magnetostrictive shell includes depositing piezoelectric material on the magnetostrictive shell to a thickness of up to about 5 nm.

In some aspects, the techniques described herein relate to a method wherein the piezoelectric shell includes one of barium titanate (BaTiO3), lead zirconate titanate (PZT) (Pb[ZrxTi1−x]O3 (0≤x≤1)), lead titanate (PbTiO3), or barium-calcium zirconium titanate (BCZT).

In some aspects, the techniques described herein relate to a method wherein the anisotropic magnetoelectric nanoparticle is one of a plurality of anisotropic magnetoelectric nanoparticles and further including dispersing the plurality of anisotropic magnetoelectric nanoparticles in a biocompatible solvent.

In some aspects, the techniques described herein relate to an anisotropic magnetoelectric nanoparticle including a magnetite core having a maximum lateral dimension of about 170 nm to about 550 nm and a maximum thickness of up to 50 nm, a magnetostrictive shell on the magnetite core, and a piezoelectric shell on the magnetostrictive shell.

In some aspects, the techniques described herein relate to an anisotropic magnetoelectric nanoparticle wherein the magnetostrictive shell has a thickness of about 5 nm and the piezoelectric shell has a thickness of about 5 nm.

In some aspects, the techniques described herein relate to an anisotropic magnetoelectric nanoparticle wherein the magnetostrictive shell includes an AxFe3−xO4, where A is one of Co, Mn, Ni, Fe, Zn, Mg, or Cu.

In some aspects, the techniques described herein relate to an anisotropic magnetoelectric nanoparticle wherein A is Co.

In some aspects, the techniques described herein relate to an anisotropic magnetoelectric nanoparticle wherein the piezoelectric shell includes one of barium titanate (BaTiO3), lead zirconate titanate (PZT) (Pb[ZrxTi1−x]O3 (0≤x≤1)), lead titanate (PbTiO3), or barium-calcium zirconium titanate (BCZT).

In some aspects, the techniques described herein relate to an anisotropic magnetoelectric nanoparticle wherein the piezoelectric shell is barium titanate (BaTiO3).

In some aspects, the techniques described herein relate to an anisotropic magnetoelectric nanoparticle wherein the anisotropic magnetoelectric nanoparticle has a magnetoelectric coupling coefficient of about 130-180 mV/mT-cm under a 220 mT, 150 Hz alternating current magnetic field.

In some aspects, the techniques described herein relate to an anisotropic magnetoelectric nanoparticle wherein the magnetite core is hexagonal.

In some aspects, the techniques described herein relate to an anisotropic magnetoelectric nanoparticle including an anisotropic magnetite core, a layer of AxFe3−xO4 disposed on the magnetite core, where A is one of Co, Mn, Ni, Fe, Zn, Mg, or Cu, and a layer of piezoelectric material disposed on the layer of AₓFe3−xO4.

In some aspects, the techniques described herein relate to an anisotropic magnetoelectric nanoparticle wherein the anisotropic magnetoelectric nanoparticle has a magnetoelectric coupling coefficient of about 130-180 mV/mT-cm under a 220 mT, 150 Hz alternating current magnetic field.

All combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. Terminology explicitly employed herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1A is an illustration of neuromodulation mediated by a magnetoelectric nanodisc (MEND).

FIG. 1B is a transmission electron microscopy (TEM) image of a magnetite (Fe₃O₄) nanodisc (MND) that forms the core in a MEND. The scale bar is 100 nm. The inset shows a selected area electron diffraction (SAED) pattern revealing epitaxial growth. The scale bar on the inset is 10 nm⁻¹.

FIG. 1C is a TEM image of an MND surrounded by a CoFe₂O₄ shell (a core-shell Fe₃O₄—CoFe₂O₄ nanodisc, or CFOND). The inset shows a SAED pattern revealing epitaxial growth. The scale bar on the inset is 10 nm⁻¹.

FIG. 1D is a scanning electron microscopy (SEM) image of core-double shell Fe₃O₄—CoFe₂O₄—BaTiO₃ MENDs. Scale bar=100 nm.

FIG. 1E is an illustration of the electrochemical measurement apparatus that employs surface charge variation of MENDs in response to applied magnetic field (MF) to determine the magnetoelectric coefficient (α_{ME}).

FIG. 1F is an illustration showing simulated magnetostriction constant (bars) and magnetostrictive displacement maps (framed insets) for hexagonal CFONDs (left), hexagonal MNDs (middle), and Fe₃O₄—CoFe₂O₄ core-shell spherical nanoparticles (right). The volume of the particles was fixed across conditions. Inset: Color index for direction in the magnetostrictive displacement maps.

FIG. 1G shows an image of an electric polarization generated in BaTiO₃ shells deposited onto CFONDs upon exposure to an offset magnetic field (OMF) of 220 mT and an alternating magnetic field (AMF) with an amplitude of 10 mT.

FIG. 1H is a graph of the α_{ME} at an AMF with a frequency f_{AMF}=150 Hz and amplitude H_{AMF}=10 mT measured at varying magnitudes of OMF for MENDs (circles), isotropic magnetoelectric nanoparticles (MENPs, squares), and CFONDs (triangles).

FIG. 1I is a graph of α_{ME} for MENDs as a function of AMF frequency at H_{AMF}=10 mT and OMF magnitude 220 mT.

FIG. 1J is a graph of the α_{ME} for MENDs as a function of AMF amplitude for f_{AMF}=150 Hz and OMF magnitude 220 mT.

FIG. 1K is a drawing of a cross-section of a CFOND.

FIG. 1L is a drawing of a cross-section of an inner shell with multiple layers suitable for use with a CFOND (e.g., in the dotted rectangle in FIG. 1C) and/MEND.

FIG. 1M is a drawing of an inner shell with multiple layers with individual layers having different stoichiometries suitable for use with a CFOND (e.g., in the dotted rectangle in FIG. 1C) and/MEND.

FIG. 1N is another image of a cross-section of the inner shell marked with the dotted square in FIG. 1C illustrating an inner shell with multiple layers with individual layers having different compositions.

FIG. 2A shows relative GCaMP6s fluorescence change (ΔF/F₀) in hippocampal neurons decorated with MENDs before (left) and after (right) magnetic field application. Scale bars: 150 μm.

FIG. 2B shows individual (top) and average (bottom) of the relative GCaMP6s fluorescence change (ΔF/F₀) of the hippocampal neurons in response to 10 mT AMF with 1 kHz frequency and 220 mT OFM.

FIG. 2C shows individual traces of GCaMP6s ΔF/F₀ in 300 hippocampal neurons decorated with MENDs in response to 10 mT AMF with frequencies 100, 150, 250, 500, and 1000 Hz (OMF magnitude 220 mT). The first and second dashed lines for each frequency indicate the beginning and end of MF stimulation, respectively.

FIG. 2D shows a graph of the average traces of GCaMP6s ΔF/F₀ in 300 hippocampal neurons decorated with MENDs in response to 10 mT AMF with frequencies 100, 150, 250, 500, and 1000 Hz (OMF magnitude 220 mT). The first and second dashed lines indicate the beginning and end of MF stimulation, respectively.

FIG. 2E shows individual cell (top) and mean (bottom) GCaMP6s fluorescence changes in 300 neurons in response to 2-second MF epochs applied at varying intervals (OMF 220 mT; AMF 150 Hz, 10 mT).

FIG. 2F shows a graph of the number of GCaMP6s fluorescence peaks as a function of stimulation epoch length for rest intervals of 10, 30, 60, 90, and 120 seconds (OMF 220 mT; AMF 150 Hz, 10 mT).

FIG. 2G shows individual cell (top) and mean (bottom) GCaMP6s fluorescence changes in response to 2-second MF epochs at 40 second intervals for 0.75 μg mm⁻² MEND density.

FIG. 2H is a fluorescent image of a Live-Dead assay in neurons before and after 3 cycles of MF at a 1 μg mm⁻² MEND density.

FIG. 2I is a fluorescent image of a Live-Dead assay in neurons before and after 3 cycles of MF at a 0.75 μg mm⁻² MEND density.

FIG. 2J is a graph showing the change in live cell ratio (normalized to the total number of the cells counted with Hoest staining) following 3 cycles of MF for neurons decorated with different MEND densities. Statistical significance was tested via one-way ANOVA and Tukey's multiple comparison tests (n=5 plates per condition, P=3.79×10⁻⁷ for 1 μg mm⁻². P=0.79 for 0.794 μg mm⁻²; P=0.998 for 0.75 μg mm⁻²; ****P<0.0001, n.s. P>0.05).

FIG. 3A is an illustration of the stimulation mechanism combining cable model and repeated excitation of neural activity, where d is spacing between MEND particles, a is cell radius, ΔV is the change in membrane potential per half-period of an AMF, and V₀ is the voltage generated by a single MEND.

FIG. 3B shows the calculated membrane potential change ΔV for every half-period of an AMF, as a function of V₀ and d.

FIG. 3C is a graph of the simulated membrane potential V(t) after AMF onset to activate MENDs with V₀=0.03 mV for varying a values. The threshold potential for action potential firing, −55 mV, is indicated with the dashed line.

FIG. 3D is a graph of the simulated membrane potential at a time t=2 seconds after onset of magnetic field as a function of d, for varying V₀ values. V₀=0.03 mV is the measured value for MEND particles in this study.

FIG. 3E is a graph showing the time to reach threshold membrane potential (−55 mV) from the resting potential (−75 mV) as a function of AMF frequency f_{AMF} for varying V₀ values.

FIG. 3F are SEM images showing MENDs decorating cultured hippocampal neurons. (ii—middle panel) A higher magnification image of the area marked by a box in the panel (i—left panel). (iii—right panel) MENDs on the neuron surface are identified by the presence of iron, titanium, and barium in energy dispersive X-ray spectroscopy. Scale bars: 20 μm (I—left panel), 5 μm (ii—middle panel), 100 nm (iii—right panel).

FIG. 3G shows GCaMP6s fluorescence changes in neurons decorated with MEND following 2-second stimulations (OMF 220 mT; AMF 150 Hz, 10 mT, marked by vertical grey bars) in the presence of tetrodotoxin (TTX, 1 μM).

FIG. 3H shows GCaMP6s fluorescence changes in neurons decorated with MEND following 2-second stimulations (OMF 220 mT; AMF 150 Hz, 10 mT, marked by vertical grey bars) in the presence of a cocktail of (2R)-amino-5-phosphonovaleric acid (AP5, 100 μM) and 6-cyano-7-nitroquinoxaline-2,3-dione (CNQX, 20 μM).

FIG. 4A is a schematic of MEND-mediated neuromodulation. MEND particles were injected into the mouse ventral tegmental area (VTA), and the mice were placed inside a permanent magnet field providing OMF of 220 mT and a surrounding solenoid providing AMF with an amplitude of 10 mT and a frequency of 150 Hz.

FIG. 4B are confocal images of c-Fos-expressing neurons among DAPI-marked cells in the VTA. Top left: MENDs (1.5 mg/ml) with (+) magnetic stimulation (ON); top right: MEND particles (1.5 mg/ml) without (−) magnetic stimulation (OFF); bottom left: control MNDs (1.5 mg/ml)+magnetic stimulation; bottom right: MENDs (0.5 mg/ml)+magnetic stimulation. Scale bars are 25 μm.

FIG. 4C is a graph showing the quantification of c-Fos positive neurons for the conditions shown in (FIG. 4B) as well as the subjects injected with phosphate buffered saline (PBS) and exposed to magnetic field. Statistical significance was tested via one-way ANOVA and Tukey's multiple comparison tests (n=6 mice per condition, VTA F_{3,20}=93.2 P=1.21×10⁻¹²; mPFC F_{3,20}=60.81 P=1.21×10⁻¹; VTA F_{3,20}=62.03 P=1.29×10⁻¹²; ****P<0.0001).

FIG. 4D shows confocal images of c-Fos positive neurons in the medial prefrontal cortex (mPFC) for the same conditions shown in (FIG. 4B) and (FIG. 4C). Scale bars are 100 μm.

FIG. 4E is a graph showing the quantification of c-Fos positive neurons in the medial prefrontal cortex (mPFC) for the same conditions shown in (FIG. 4B) and (FIG. 4C). Statistical significance was tested via one-way ANOVA and Tukey's multiple comparison tests (n=6 mice per condition, VTA F_{3,20}=93.2 P=1.21×10⁻¹²; mPFC F_{3,20}=60.81 P=1.21×10⁻¹¹; VTA F_{3,20}=62.03 P=1.29×10⁻¹²; ****P<0.0001).

FIG. 4F shows confocal images of c-Fos positive neurons in the nucleus accumbens (NAc) for the same conditions shown in FIG. 4B and FIG. 4C. Scale bars are 100 μm. Statistical significance was tested via one-way ANOVA and Tukey's multiple comparison tests (n=6 mice per condition, VTA F_{3,20}=93.2 P=1.21×10⁻¹²; mPFC F_{3,20}=60.81 P=1.21×10⁻¹¹; VTA F_{3,20}=62.03 P=1.29×10⁻¹²; ****P<0.0001).

FIG. 4G is a graph showing the quantification of c-Fos positive neurons in the nucleus accumbens (NAc) for the same conditions shown in FIG. 4B and FIG. 4C.

FIG. 4H shows representative confocal images showing expression of GCaMP6s in the VTA. Scale bar: 50 μm.

FIG. 4I is a fiber photometry recording of relative GcaMP6s fluorescence change (ΔF/F₀) in the VTA of anaesthetized mice injected with MENDs in the same region. (Top) Individual trial ΔF/F₀. (Bottom) Average ΔF/F₀ across trials shown above. Solid line represents mean and shaded areas mark s.e.m. (n=4 mice, 10-15 trials per mouse). The box indicates magnetic field epochs (2 seconds, OMF 220 mT, AMF 10 mT, 150 Hz).

FIG. 4J is a schematic of the place preference behavioral apparatus (top) and experimental timeline (bottom).

FIG. 4K is a graph of the time spent in the stimulation chamber out of a total test time of 600 seconds, for pre- (Day 1, open markers) and post- (Day 5, solid markers) learning. Paired t-test was performed for MEND (n=11) and MND (n=7) groups, and Wilcoxon signed-rank test was performed for PBS (n=7) group because the data did not follow normal distribution. ***P≤0.001, **P≤0.01, *P≤0.05, n.s. P>0.05).

FIG. 4L is a graph of the change in time spent in stimulation chamber between Day 1 and Day 5. (One-way ANOVA with Tukey's post-hoc comparison test, ***P≤0.001, **P≤0.01, *P≤0.05, n.s. P>0.05.)

FIG. 5A is a transmission electron microscopy (TEM) image of an ensemble of hematite (Fe₂O₃) nanodiscs produced via a hydrothermal process prior to reduction.

FIG. 5B is an electron diffraction pattern of an area on a single hematite nanodisc.

FIG. 6 is an X-ray diffraction spectra of Fe₃O₄ magnetic nanodiscs (MNDs, bottom trace), Fe₃O₄—CoFe₂O₄ core-shell nanodiscs (CFONDs, middle trace), and co-double shell magnetoelectric Fe₃O₄—CoFe₂O₄—BaTiO₃ nanodiscs (MENDs, top trace) shown together with the reference spectra for Fe₃O₄ (middle bottom vertical lines, cubic inverse spinel structure), CoFe₂O₄ (left bottom vertical lines, cubic inverse spinel structure) and BaTiO₃ (right bottom vertical lines, tetragonal crystal system with perovskite structure).

FIG. 7A is a cross-sectional TEM image of a core-shell Fe₃O₄—CoFe₂O₄ nanodisc (CFOND). Scale bar=50 nm.

FIG. 7B is a cross-sectional scanning TEM (STEM) image of a CFOND at different magnifications demonstrating the epitaxial interface between Fe₃O₄ and CoFe₂O₄ layers. Scale bar is 10 nm.

FIG. 7C is a cross-sectional scanning TEM (STEM) image of a CFOND at different magnifications demonstrating the epitaxial interface between Fe₃O₄ and CoFe₂O₄ layers. Scale bar is 5 nm.

FIG. 7D is a higher magnification SEM image of the area marked with the top left rectangle in FIG. 7C. Scale bar is 2 nm.

FIG. 7E is a higher magnification SEM image of the area marked with the bottom right rectangle in FIG. 7C. Scale bar is 2 nm.

FIG. 8A is a cross-sectional TEM image of a core-double shell Fe₃O₄—CoFe₂O₄—BaTiO₃ magnetoelectric nanodisc (MEND). Scale bar is 100 nm.

FIG. 8B is a magnified view of the area marked by a rectangle in (FIG. 8A). Scale bar is 20 nm.

FIG. 8C is a STEM image demonstrating the crystal structure of BaTiO₃, (small right rectangle) which is distinct from that of Fe₃O₄ or CoFe₂O₄ (inverse spinel, large left rectangle). Scale bar is 100 nm.

FIG. 8D is a fast Fourier transform of an image of a Fe₂O₄—CoFe₂O₄ layer (large left rectangle in FIG. 8C) of the corresponding STEM image in FIG. 8C. Scale bar is 20 nm.

FIG. 8E is a fast Fourier transform of an image of a BaTiO₃ layer (small right rectangle in FIG. 8C) of the corresponding STEM image in FIG. 8C. Scale bar is 5 nm.

FIG. 9 is a schematic of a magnetoelectric coefficient (α_{ME}) measurement apparatus. To generate the magnetic field, TEMCo 14 AWG copper magnet wire was wound around a horseshoe-shaped magnetic core with a 0.5-inch gap. The coil was connected to a power supply (CROWN DC-300A Series II) and signal generator (PICOSCOPE 2204A) to generate a compound magnetic field combining an offset magnetic field (OMF, 0-320 mT) with an alternating magnetic field (AMF, 0-1 kHz, 0-14 mT). A nuclear magnetic resonance (NMR) glass tube (8 mm diameter) was used as the electrochemical cell, which was placed into the 8 mm gap of the horseshoe coil. In the cell, the Ag/AgCl reference electrode, Pt counter electrode, and the working electrode were immersed in the Tyrode solution.

FIG. 10A is a magnetic force microscopy (MFM) image of isolated CFONDs. Isolated CFONDs assume the magnetic vortex ground state magnetization.

FIG. 10B is a magnetic force microscopy (MFM) image of overlapping CFONDs. Overlapping CFONDs lose the vortex state in favor of in-plane magnetization.

FIG. 10C is an atomic force microscopy (AFM) image of isolated CFONDs corresponding to the MFM image in FIG. 10A.

FIG. 10D is an atomic force microscopy (AFM) image of the overlapping CFONDs corresponding to the MFM image in FIG. 10B.

FIG. 11A is a finite element simulation (COMSOL Multiphysics) of the electric polarization in the piezoelectric BaTiO₃ shell based on the implementation of the RMS deformation shown in FIG. 1F to the saturation magnetization parameter of the core for a hexagonal nanodisc with a 100% Fe₃O₄ magnetostrictive core and a 5 nm piezoelectric BaTiO₃ shell.

FIG. 11B is a finite element simulation (COMSOL Multiphysics) of the electric polarization in the piezoelectric BaTiO₃ shell based on the implementation of the RMS deformation shown in FIG. 1F to the saturation magnetization parameter of the core for an isotropic nanoparticle with a magnetostrictive core with a volumetric ratio 92% Fe₃O₄ and 8% CoFe₂O₄ and a 5 nm piezoelectric BaTiO₃ shell.

FIG. 12A is an SEM image of isotropic CoFe₂O₄ nanoparticles with an average diameter of 25 nm. Scale bar=100 nm.

FIG. 12B is an SEM image core-shell CoFe₂O₄—BaTiO₃ nanoparticles produced from cores in FIG. 12A. Scale bar=100 nm.

FIG. 13A is an image of topography of MENDs observed via piezoresponse force microscopy. the

FIG. 13B is an image of piezoresponse amplitude of MENDs observed via piezoresponse force microscopy. the

FIG. 13C is an image of phase of MENDs observed via piezoresponse force microscopy. the

FIG. 13D is a Piezoresponse phase curve.

FIG. 13E is a Piezoresponse amplitude curve.

FIG. 14A shows a vibrating sample magnetometry (VSM) measurement of MNDs, CFONDs, and MENDs dispersed in water.

FIG. 14B shows a VSM measurement of MNDs, CFONDs, and MENDs in a dried pellet form.

FIG. 15A is a graph showing the changes in potential corresponding to pulses of AC magnetic field with an amplitude of 10 mT and frequencies 25, 50, 75, 100, 125, 150, 250, 500, 1000, and 2000 Hz.

FIG. 15B is a graph showing the changes in potential corresponding to pulses of AC magnetic field with a frequency of 150 Hz and amplitudes 1, 2, 3, 4, 5, 6, 7, 8, 9, 10, 11, and 12 mT. Multiple repetitions (n=3) of these measurements were conducted to generate data displayed in FIGS. 11 and 1J. The boxes indicate when the magnetic field was applied.

FIG. 15C is a graph of α_{ME} at an AMF with a frequency f_{AMF}=1 kHz and amplitude H_{AMF}=10 mT measured at varying magnitudes of OMF for MENDs in Tyrode (circle points), PBS 1× (triangle points), and PBS 10× (square points).

FIG. 15D is a graph of the non-faradaic current in cyclic voltammetry (CV) at 25 mV/s scan rate, which implies the electric double layer capacitance within different electrolytes from the Equation I=dQ/dt=d(C_{dl}−E)/dt=C_{dl}ν, where I is non-faradaic current, Q is charge, t is time, C_{dl} is electric-double layer capacitance, E is potential, and v is the scan rate of Cv.

FIG. 16A is a trace of GCaMP6s ΔF/F₀ of individual (top) and average (bottom) of hippocampal neurons (n=5 culture plates) decorated with MENDs in response to 10 mT AMF with frequencies 30, 60, 100, 150, 250, 500, and 1000 Hz (OMF magnitude 220 mT). The shaded bars indicate MF epochs.

FIG. 16B is a graph showing the latency of the peak of GCaMP6s fluorescence transient relative to the start of MF epoch. The lines represent the average and standard deviation.

FIG. 16C is a graph showing the extent of neuronal response to the MF application.

FIG. 17A is a trace of population (top panel, 300 neurons from 3 plates) and average ΔF/F₀ (bottom panel) of GCaMP6s signals for neurons decorated with MENDs and subjected to magnetic field epochs of 0.5 seconds at intervals of 120 seconds, 90 seconds, 60 seconds, 30 seconds, 10 seconds). The vertical lines mark magnetic field epochs (220 mT OMF; 150 Hz, 10 mT AMF).

FIG. 17B is a trace of population (top panel, 300 neurons from 3 plates) and average ΔF/F₀ (bottom panel) of GCaMP6s signals for neurons decorated with MENDs and subjected to magnetic field epochs of 1 second at intervals of 120 seconds, 90 seconds, 60 seconds, 30 seconds, 10 seconds). The vertical lines mark magnetic field epochs (220 mT OMF; 150 Hz, 10 mT AMF).

FIG. 17C is a trace of population (top panel, 300 neurons from 3 plates) and average ΔF/F₀ (bottom panel) of GCaMP6s signals for neurons decorated with MENDs and subjected to magnetic field epochs of 5 seconds at intervals of 120 seconds, 90 seconds, 60 seconds, 30 seconds, 10 seconds). The vertical lines mark magnetic field epochs (220 mT OMF; 150 Hz, 10 mT AMF).

FIG. 17D is a trace of population (top panel, 300 neurons from 3 plates) and average ΔF/F₀ (bottom panel) of GCaMP6s signals for neurons decorated with MENDs and subjected to magnetic field epochs of 10 seconds at intervals of 120 seconds, 90 seconds, 60 seconds, 30 seconds, 10 seconds). The vertical lines mark magnetic field epochs (220 mT OMF; 150 Hz, 10 mT AMF).

FIG. 18A is a GCaMP6s calcium (Ca²⁺) image of 300 primary hippocampal neurons (n=3 culture plates) with a MEND density of 0.5 μg/mm² Each row in the top panel represents a neuron, the horizontal bar marks fluorescence intensity change ΔF normalized to baseline fluorescence F₀ (averaged between 10-30 seconds). The bottom panels show the average ΔF/F₀ (line) and standard error (shaded area). Vertical boxes mark magnetic field epochs (MF, 2 s, 220 mT DC, 150 hz and 10 mT AC).

FIG. 18B is a GCaMP6s calcium (Ca²⁺) image of 300 primary hippocampal neurons (n=3 culture plates) with a MEND density of 0.25 μg/mm². Each row in the top panel represents a neuron, the horizontal bar marks fluorescence intensity change ΔF normalized to baseline fluorescence F₀ (averaged between 10-30 seconds). The bottom panels show the average ΔF/F₀ (line) and standard error (shaded area). Vertical boxes mark magnetic field epochs (MF, 2 s, 220 mT DC, 150 hz and 10 mT AC).

FIG. 18C is a GCaMP6s calcium (Ca²⁺) image of 300 primary hippocampal neurons (n=3 culture plates) with a MEND density of 0 μg/mm². Each row in the top panel represents a neuron, the horizontal bar marks fluorescence intensity change ΔF normalized to baseline fluorescence F₀ (averaged between 10-30 seconds). The bottom panels show the average ΔF/F₀ (line) and standard error (shaded area). Vertical boxes mark magnetic field epochs (MF, 2 s, 220 mT DC, 150 Hz and 10 mT AC).

FIG. 19A is a live-dead assay of neurons decorated with 1 μg mm⁻²before three cycles of MFs. Top left—linve cell, Top right—dead cells, Bottom left—nuclei, Scale bar=150 μm.

FIG. 19B is a live-dead assay of neurons decorated with 1 μg mm⁻² after three cycles of MFs. Top left—linve cell, Top right—dead cells, Bottom left—nuclei, Scale bar=150 μm.

FIG. 19C is a live-dead assay of neurons decorated with 0.75 μg mm⁻² before three cycles of MFs. Top left—linve cell, Top right—dead cells, Bottom left—nuclei, Scale bar=150 μm.

FIG. 19D is a live-dead assay of neurons decorated with 0.75 μg mm⁻² after three cycles of MFs. Top left—linve cell, Top right—dead cells, Bottom left—nuclei, Scale bar=150 μm.

FIG. 19E is a live-dead assay of neurons decorated without MENDs before three cycles of MFs. Top left—linve cell, Top right—dead cells, Bottom left—nuclei, Scale bar=150 μm.

FIG. 19F is a live-dead assay of neurons decorated without MENDs and after three cycles of MFs. Top left—linve cell, Top right—dead cells, Bottom left—nuclei, Scale bar=150 μm.

FIG. 19G is a graph of live cell numbers normalized with the number of total cells before MF application on neurons decorated with MENDs at densities of 1 μg mm⁻², 0.75 μg mm⁻², and without MENDs. Statistical significance was tested via Kruskal-Wallis ANOVA and Tukey's multiple comparison tests (n=5 plates per condition, P=0.515 (before) and 6.98×10⁻⁵ (after) for 1 vs 0.75 μg mm⁻²; P=0.278 (before) and 1.78×10⁻⁵ (after) for 0.75 vs 0 μg mm⁻², P=0.881 (before) and 0.598 (after) for 0.75 vs 0 μg mm⁻²; ****P≤0.0001, n.s. P>0.05).

FIG. 19H is a graph of live cell numbers normalized with the number of total cells after MF application on neurons decorated with MENDs at densities of 1 μg mm⁻², 0.75 μg mm⁻², and without MENDs. Statistical significance was tested via Kruskal-Wallis ANOVA and Tukey's multiple comparison tests (n=5 plates per condition, P=0.515 (before) and 6.98×10⁻⁵ (after) for 1 vs 0.75 μg mm⁻²; P=0.278 (before) and 1.78×10⁻⁵ (after) for 0.75 vs 0 μg mm⁻², P=0.881 (before) and 0.598 (after) for 0.75 vs 0 μg mm⁻²; ****P≤0.0001, n.s. P >0.05).

FIG. 20A shows GCaMP6s calcium (Ca²⁺) imaging of 300 primary hippocampal neurons (n=3 culture plates) transfected to express TRPV1 thermoreceptors.

FIG. 20B is an image of neurons expressing GCaMP6s (top left), TRPV1 (top right), and their overlay (bottom left). Scale bars are 150 μm.

FIG. 20C shows Ca imaging with neurons transfected to express TRPV4 putative mechanoreceptors.

FIG. 20D is an image of neurons expressing GCaMP6s (top left), TRPV4 (top right), and their overlay (bottom left). Scale bars are 150 μm.

FIG. 21A is an illustration of the classic cable model in the presence of multiple voltage sources. Here d is the inter-electrode or inter-particle distance, a is the axon radius, and V₀ is the potential applied by an electrode or generated by an individual MEND.

FIG. 21B is an illustration of an analogous model using MENDs as voltage sources. Here d is the inter-electrode or inter-particle distance, a is the axon radius, and V₀ is the potential applied by an electrode or generated by an individual MEND.

FIG. 22A is an SEM image of primary hippocampal neurons (at 10 days in vitro) without any nanoparticles at 200× (Scale bar=50 μm)

FIG. 22B is an SEM image of primary hippocampal neurons (at 10 days in vitro) without any nanoparticles at 4000× magnification. (Scale bars=2 μm).

FIG. 23A is an SEM image of hippocampal neurons (10 days) decorated with MENDs. Scale bar=500 nm.

FIG. 23B is a merged image of carbon (C, also shown in FIG. 23C), iron (Fe, also shown in FIG. 23D), barium (Ba, also shown in FIG. 23E), and titanium (Ti, also shown in FIG. 23F) atomic maps of the image shown in FIG. 23A. Scale bar=500 nm.

FIG. 23C is an energy-dispersive X-ray spectroscopy atomic map of the carbon (C) portion of the image shown in FIG. 23A. Scale bar=500 nm.

FIG. 23D is an energy-dispersive X-ray spectroscopy atomic map of iron (Fe) portion of the image shown in FIG. 23A. Scale bar=500 nm.

FIG. 23E is an energy-dispersive X-ray spectroscopy atomic map of barium (Ba) portion of the image shown in FIG. 23A. Scale bar=500 nm.

FIG. 23F is an energy-dispersive X-ray spectroscopy atomic map of titanium (Ti) portion of the image shown in FIG. 23A. Scale bar=500 nm.

FIG. 24A is a plot of GCaMP6s ΔF/F₀ in 300 neurons (n=3 plates) treated with tetrodotoxin (TTX, 1 μM) corresponding to the average ΔF/F₀ plots shown in FIGS. 3G and 3H. Vertical bars mark magnetic field epochs (MF, 2 seconds, 220 mT OMF; 10 mT, 150 Hz AMF).

FIG. 24B is a plot of GCaMP6s ΔF/F₀ in 300 neurons (n=3 plates) treated with 6-a cyano-7-nitroquinoxaline-2,3-dione (CNQX, 20 μM) and (2R)-amino-5-phosphonovaleric acid (AP5, 100 μM) cocktail corresponding to the average ΔF/F₀ plots shown in FIGS. 3G and 3H.Vertical bars mark magnetic field epochs (MF, 2 seconds, 220 mT OMF; 10 mT, 150 Hz AMF).

FIG. 25A is a confocal micrograph of c-Fos expression visible across the entire brain slice, with distinct increase in the VTA on the side injected with MENDs (1.5 μL at 1.5 mg/mL, marked with an arrow) following exposure to magnetic field (220 mT DC, 150 Hz, 10 mT AC; three 2-second epochs separated by 90-second rest epochs). Scale bar=3 mm.

FIG. 25B shows higher resolution (20×) confocal images of nuclear stain DAPI (left), c-Fos (middle), and their overlay (right) in brain slices a mouse injected with MENDs and exposed to magnetic field. Scale bar=25 μm.

FIG. 25C shows higher resolution (20×) confocal images of nuclear stain DAPI (left), c-Fos (middle), and their overlay (right) in brain slices from a mouse injected with MENDs and not exposed to magnetic field. Scale bar=25 μm.

FIG. 25D shows higher resolution (20×) confocal images of nuclear stain DAPI (left), c-Fos (middle), and their overlay (right) in brain slices from a mouse injected with control Fe₃O₄ nanodiscs (MNDs, 1.5 μL at 1.5 mg/mL) and exposed to magnetic field. Scale bar=25 μm.

FIG. 25E shows higher resolution (20×) confocal images of nuclear stain DAPI (left), c-Fos (middle), and their overlay (right) in brain slices from a mouse injected with PBS (1.5 μL) and exposed to magnetic field. Scale bar=25 μm.

FIG. 25F shows higher resolution (20×) confocal images of nuclear stain DAPI (left), c-Fos (middle), and their overlay (right) in brain slices from a mouse injected with a low concentration of MENDs (1.5 μL at 0.5 mg/mL) and exposed to magnetic field. Scale bar=25 μm.

FIG. 26A shows confocal images of c-Fos expression in the nucleus accumbens (NAc) for a mouse injected with MENDs (1.0 mg/mL, 1.5 μL) and exposed to magnetic field (220 mT OMF; 150 Hz, 10 mT AMF, three 2-second epochs separated by 90-second rest epochs). Scale bar=100 μm.

FIG. 26B shows confocal images of c-Fos expression in the NAc for a mouse injected with MENDs and not exposed to magnetic field. Scale bar=100 μm.

FIG. 26C shows confocal images of c-Fos expression in the NAc for a mouse injected with a low concentration of MENDs (0.5 mg/mL, 1.5 μL) and exposed to magnetic field. Scale bar=100 μm.

FIG. 26D shows confocal images of c-Fos expression in the NAc for a mouse injected with PBS (1.5 μL) and exposed to magnetic field. Scale bar=100 μm.

FIG. 26E shows confocal images of c-Fos expression in the NAc for a mouse injected with control MNDs (1.0 mg/mL, 1.5 μL) and exposed to magnetic field. Scale bar=100 μm.

FIG. 27A shows confocal images of c-Fos expression in the medial prefrontal cortex (mPFC) for a mouse injected with MENDs (1.0 mg/mL, 1.5 μL) and exposed to magnetic field (220 mT OMF; 150 Hz, 10 mT AMF, three 2-second epochs separated by 90-second rest epochs). Scale bar=100 μm.

FIG. 27B shows confocal images of c-Fos expression in the mPFC for a mouse injected with MENDs and not exposed to magnetic field. Scale bar=100 μm.

FIG. 27C shows confocal images of c-Fos expression in the mPFC for a mouse injected with a low concentration of MENDs (0.5 mg/mL, 1.5 μL) and exposed to magnetic field. Scale bar=100 μm.

FIG. 27D shows confocal images of c-Fos expression in the mPFC for a mouse injected with PBS (1.5 μL) and exposed to magnetic field. Scale bar=100 μm.

FIG. 27E shows confocal images of c-Fos expression in the mPFC for a mouse injected with control MNDs (1.0 mg/mL, 1.5 μL) and exposed to magnetic field. Scale bar=100 μm.

FIG. 28A is an illustration of the fiber photometry system.

FIG. 28B is an illustration of magnetic field generation system.

FIG. 28C is a photograph of a front view of the experimental setup used for fiber photometry recordings during magnetoelectric stimulation (220 mT OMF; 150 Hz, 10 mT AMF) mediated with MENDs and the corresponding control experiments. Scale bar is 2 cm.

FIG. 28D is a photograph of a back view of the experimental setup used for fiber photometry recordings during magnetoelectric stimulation (220 mT OMF; 150 Hz, 10 mT AMF) mediated with MENDs and the corresponding control experiments. Scale bar is 2 cm.

FIG. 29A is a confocal image of the axons VTA projecting to mPFC for DaPI-stained cells Scale bar=150 μm.

FIG. 29B is a confocal image of the axons VTA projecting to mPFC (for the GCaMP6s-expressing axons. Scale bar=150 μm.

FIG. 29C is a confocal image of an overlay of DAPI and GCaMP6s signals. Scale bar=150 μm.

FIG. 29D is a confocal image of the axons VTA projecting to NAc for dAPI-stained cells. Scale bar=150 μm.

FIG. 29E is a confocal image of GCaMP6s expression of the axons VTA projecting to Nac for the GCaMP6s-expressing axons. Scale bar=150 μm.

FIG. 29F is a confocal image of an overlay of DAPI and GCaMP6s signals for the axons VTA projecting to NAc. Scale bar=150 μm.

FIG. 30A shows photometric recordings of average relative GCaMP6s fluorescence in the VTA of the mice injected with MND (1.5 mg/mL). Lines represent average and shading represents standard deviation (n=3 mice per condition; 10 trials per mouse). Vertical bars denote magnetic field epochs (2 seconds, 220 mT OMF; 150 Hz, 10 mT AMF).

FIG. 30B shows photometric recordings of average relative GCaMP6s fluorescence in the VTA of the mice injected with PBS. Lines represent average and shading represents standard deviation (n=3 mice per condition; 10 trials per mouse). Vertical grey bars denote magnetic field epochs (2 seconds, 220 mT OMF; 150 Hz, 10 mT AMF).

FIG. 31 is a photograph of the custom apparatus employed for place-preference assays enabling application of magnetic fields for magnetoelectric neuromodulation. Scale bar is 10 cm.

FIG. 32 is a summary of time spent by each subject in the stimulation chamber of the arena, out of a total test time of 600 seconds for each trial. Scale bars denote standard deviation. (n=11 for MEND group, n=7 for MND and PBS, Kruskal-Wallis test and Tukey's post- hoc comparison test, ***P<0.001, **P<0.01, *P<0.05, n.s. P>0.05). The concentration of MENDs and MNDs was 1.5 mg/mL, all injections were 1.5 μL.

FIG. 33A shows representative confocal images of expression of Iba1 in the brains of mice 2 weeks following surgery for unilateral injection with MENDs. 90 minutes before the perfusion, the animals were applied to the magnetic field (220 mT DC and 150 Hz, 10 mT AC; three 2-second epochs separated by 90-second rest epochs). Scale bars=50 μm. Two-sample t-test was performed (n=4 mice per group).

FIG. 33B shows representative confocal images of expression of Iba1 in the brains of mice 2 weeks following surgery for contralateral injection with PBS. 90 minutes before the perfusion, the animals were applied to the magnetic field (220 mT DC and 150 Hz, 10 mT AC; three 2-second epochs separated by 90-second rest epochs). Scale bars=50 μm. Two-sample t-test was performed (n=4 mice per group).

FIG. 33C is a graph showing the average percentages of expression of Iba1 in the brains of mice 2 weeks following surgery for unilateral injection with MENDs and contralateral injection with PBS. 90 minutes before the perfusion, the animals were applied to the magnetic field (220 mT DC and 150 Hz, 10 mT AC; three 2-second epochs separated by 90-second rest epochs). Two-sample t-test was performed (n=4 mice per group, P values are noted in the plots).

FIG. 33D shows representative confocal images of expression of GFAP CD68 in the brains of mice 2 weeks following surgery for unilateral injection with MENDs and after a magnetic field was applied. 90 minutes before the perfusion, the animals were applied to the magnetic field (220 mT DC and 150 Hz, 10 mT AC; three 2-second epochs separated by 90-second rest epochs). Scale bars=50 μm. Two-sample t-test was performed (n=4 mice per group).

FIG. 33E shows representative confocal images of expression of GFAP CD68 in the brains of mice 2 weeks following surgery for contralateral injection with PBS and after a magnetic field was applied. 90 minutes before the perfusion, the animals were applied to the magnetic field (220 mT DC and 150 Hz, 10 mT AC; three 2-second epochs separated by 90-second rest epochs). Scale bars=50 μm. Two-sample t-test was performed (n=4 mice per group).

FIG. 33F is a graph showing the average percentages of expression of GFAP in the brains of mice 2 weeks following surgery for unilateral injection with MENDs and contralateral injection with PBS. 90 minutes before the perfusion, the animals were applied to the magnetic field (220 mT DC and 150 Hz, 10 mT AC; three 2-second epochs separated by 90-second rest epochs). Two-sample t-test was performed (n=4 mice per group, P values are noted on the plot).

FIG. 33G is a graph showing the average percentages of expression of CD68 in the brains of mice 2 weeks following surgery for unilateral injection with MENDs and contralateral injection with PBS. 90 minutes before the perfusion, the animals were applied to the magnetic field (220 mT DC and 150 Hz, 10 mT AC; three 2-second epochs separated by 90-second rest epochs). Two-sample t-test was performed (n=4 mice per group, P values are noted on the plot).

FIG. 34A shows representative confocal images of expression across conditions of Iba1 in the brain of mice 2 weeks following surgery for unilateral MENDs injection. Scale bars=50 μm. Two-sample t-test was performed (n=4 mice per group).

FIG. 34B shows representative confocal images of expression across conditions of Iba1 in the brain of mice 2 weeks following surgery for contralateral implantation of 300 μm stainless steel microwire. Scale bars=50 μm. Two-sample t-test was performed (n=4 mice per group).

FIG. 34C is a graph showing the average percentages of expression across conditions of Iba1 in the brain of mice 2 weeks following surgery for unilateral MENDs injection and contralateral implantation of 300 μm stainless steel microwire. Two-sample t-test was performed (n=4 mice per group, P values are noted on the plot).

FIG. 34D shows representative confocal images of expression across conditions of GFAP and CD68 in the brain of mice 2 weeks following surgery for unilateral MENDs injection without application of a magnetic field. Scale bars=50 μm. Two-sample t-test was performed (n=4 mice per group).

FIG. 34E shows representative confocal images of expression across conditions of GFAP and CD68 in the brain of mice 2 weeks following surgery for contralateral implantation of 300 μm stainless steel microwire without application of a magnetic field. Scale bars=50 μm. Two-sample t-test was performed (n=4 mice per group).

FIG. 34F is a graph showing the average percentages of expression across conditions of GFAP in the brain of mice 2 weeks following surgery for unilateral MENDs injection and contralateral implantation of 300 μm stainless steel microwire. Two-sample t-test was performed (n=4 mice per group, P values are noted on the plot).

FIG. 34G is a graph showing the average percentages of expression across conditions of CD68 in the brain of mice 2 weeks following surgery for unilateral MENDs injection and contralateral implantation of 300 μm stainless steel microwire. Two-sample t-test was performed (n=4 mice per group, P values are noted on the plot).

FIG. 35A shows a simulation of a magnetic field from a helmet for magnetic generation in a clinical application. The large rectangle represents the static magnetic field and the two smaller rectangles represent AMF coils. A static magnetic field of 220 mT offset and an AMF is applied.

FIG. 35B shows a simulation of an AMF from a helmet for magnetic generation in a clinical application. The large rectangle represents the static magnetic field and the two smaller rectangles represent AMF coils. The magnetic static field is zero.

FIG. 35C is a plot of the magnetic field along the middle line of the helmet for the clinical application shown in FIG. 35A.

FIG. 36 shows a plurality of MENDs dispersed in a biocompatible solvent.

### DETAILED DESCRIPTION

### Magnetoelectric Nanodiscs (MENDs)

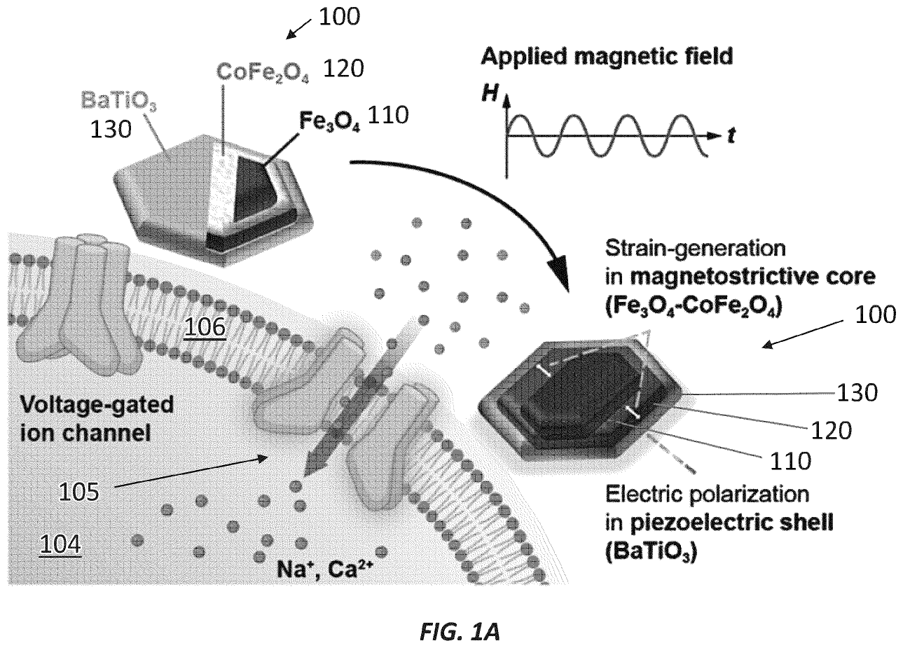

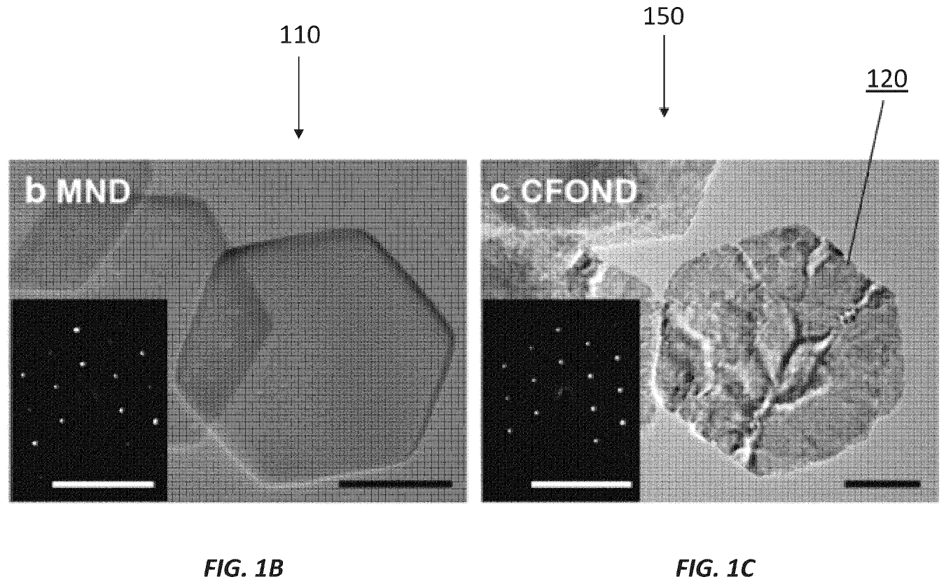

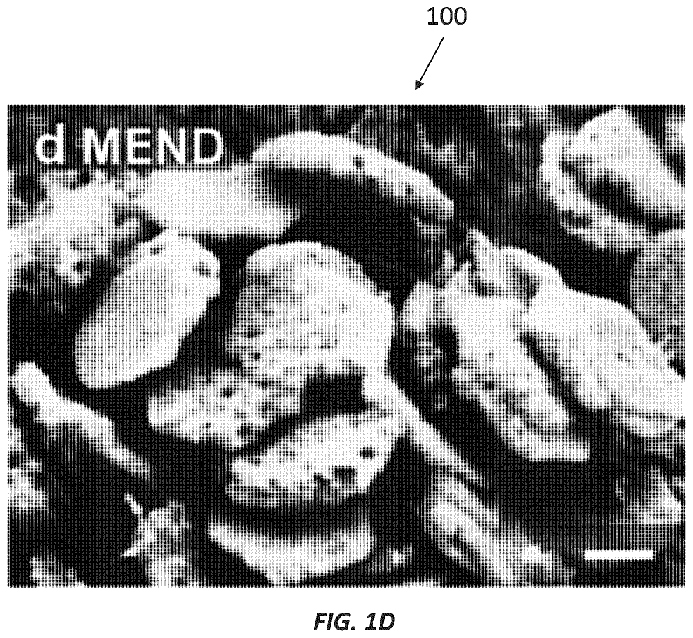

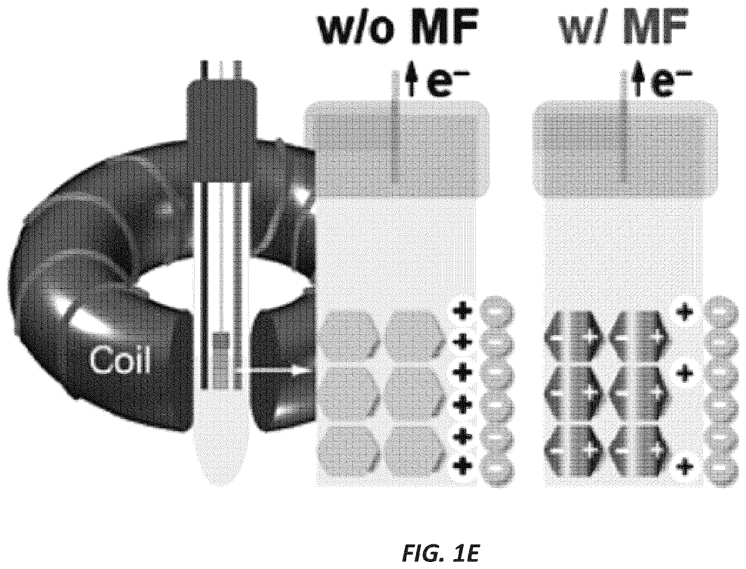

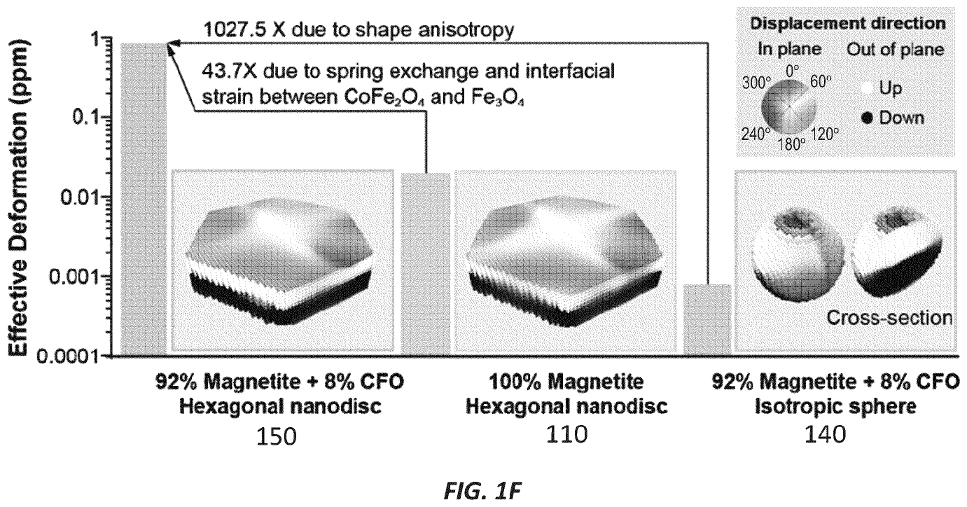

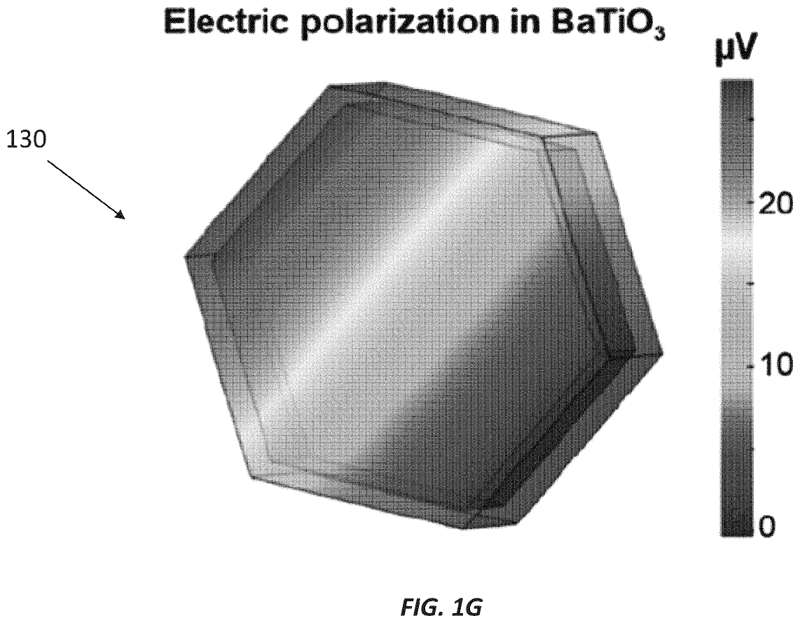

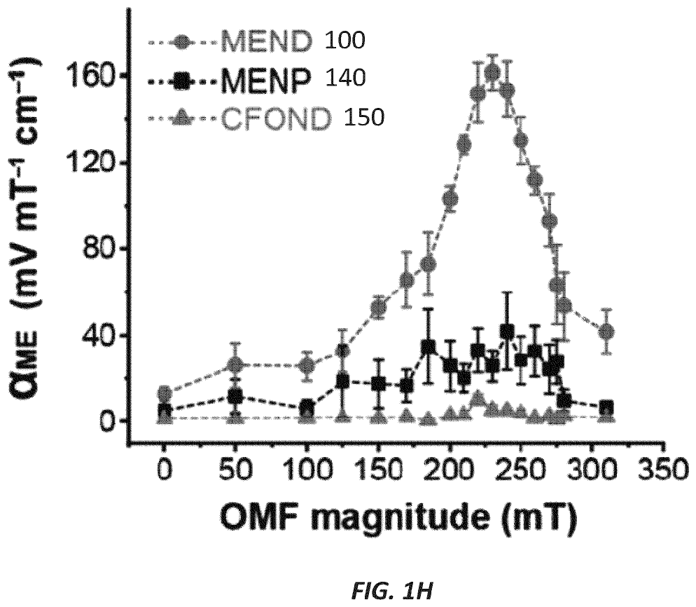

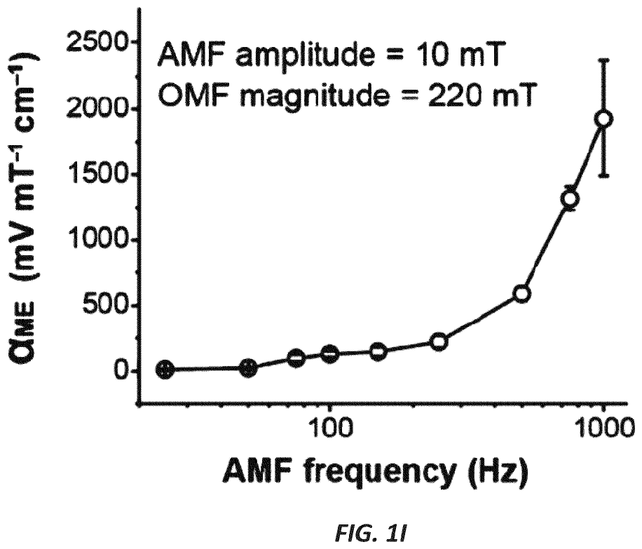

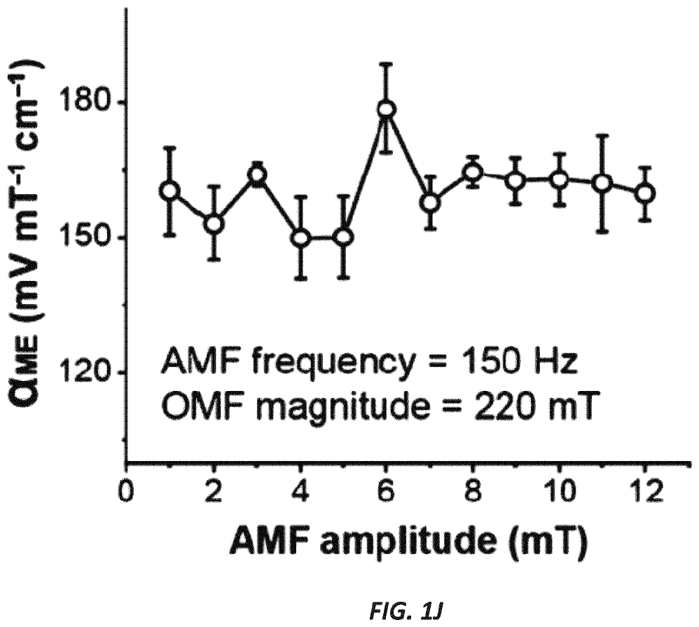

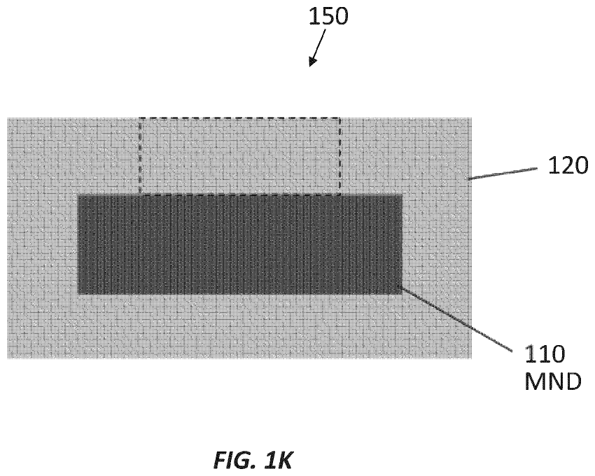

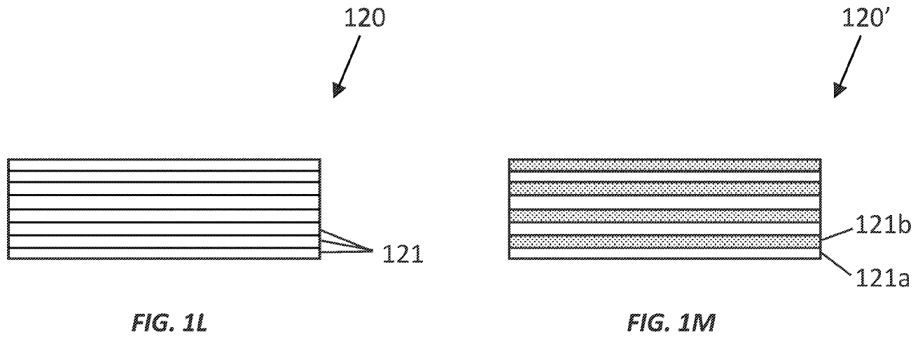

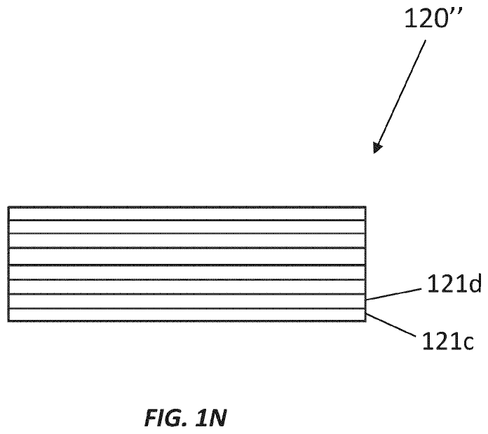

FIGS. 1A-1J illustrate inventive magnetoelectric nanodiscs (MENDs) 100 for bio-signaling and other applications. FIG. 1A shows how a MEND 100, which includes a hexagonal magnetic core (also referred to as a magnetite (Fe₃O₄) nanodisc (MND) or MND core 110) surrounded a magnetostrictive inner shell 120 and a piezoelectric outer shell 130, can be used to generate an electric polarization for opening or closing a voltage-gated ion channel 105 in a cell membrane 106.

Typically, each MND core 110 is a hexagonal disc about 20-50 nm thick with short diagonals (minimum lateral dimensions) of between about 100 nm and about 300 nm and long diagonals (maximum lateral dimensions) between about 170 and about 550 nm. The MND core's diameter can be regulated during synthesis of the hematite nanodiscs by adjusting the amount of water used for the hydrothermal process. The MND core 110 may have a maximum thickness of up to about 50 nm. The inner shell 120 and outer shells 130 may each be about 3-20 nm thick (e.g., about 5 nm thick).

Applying an AC magnetic field to the MEND 100 causes the magnetostrictive inner shell 120 to strain the piezoelectric outer shell 130, generating an electrical potential that opens the ion channel 105, allowing ions to flow through the cell membrane 106 and modulate the cell 104. This strain-mediated transduction of the magnetic field to an electrical potential at the interface between the magnetostrictive inner shell 120 (magnetic field strain) and the piezoelectric outer shell 130 (strain electric polarization) enhances the MEND's 100 magnetoelectric (ME) coupling by several orders of magnitude. For example, a MEND 100 may have an AC magnetoelectric (ME) coupling coefficient of 130-180 mV/mT-cm under a 220 mT, 150 Hz AC magnetic field. In addition, the magnetostrictive inner shell 120 prevents charge from leaking between the cores 110 of neighboring MENDs 100 through their piezoelectric outer shells 130.

Magnetoelectric (ME) modulation relies on effective transduction of an applied magnetic field (MF) into a time-varying electric field. Although multiferroic materials (e.g. Cr₂O₃, BiFeO₃) can exhibit direct magnetoelectricity, the effect is enhanced by orders of magnitude via strain-mediated coupling at the interface between a magnetostrictive material (MF to strain, e.g., Metglas alloys, Terfenol-D, or CoFe₂O₄) and a piezoelectric material (strain to electric polarization, e.g. lead zirconate titanate (PZT) or BaTiO₃).The coupling-mediated strain can be enhanced by increasing the crystalline anisotropy of the magnetostrictive material in the inner shell 120 and the MEND's 100 shape anisotropy. Ferrites (MFe₂O₄, where M=Ni, Mg, Fe, Co, Mn, or Bi) are bio-compatible magnetostrictive materials, so the crystalline and shape anisotropies can be increased at the same time by forming a crystalline-anisotropic ferrite layer—providing crystalline anisotropy—on a hexagonal magnetite (Fe₃O₄) nanodisc (MND, shown in FIG. 1B) core 110—providing crystalline anisotropy. Core-shell architectures may be advantageous in ensemble applications of ME nanomaterials, since the separation between the more conductive magnetostrictive cores may prevent leakage of charge generated in each piezoelectric shell. Additionally, magnetostriction scales with magnetic anisotropy, which is a combination of magnetocrystalline anisotropy and the shape anisotropy of the particles. Thus, anisotropic nanodiscs may exhibit greater ME coefficients than spherical counterparts.

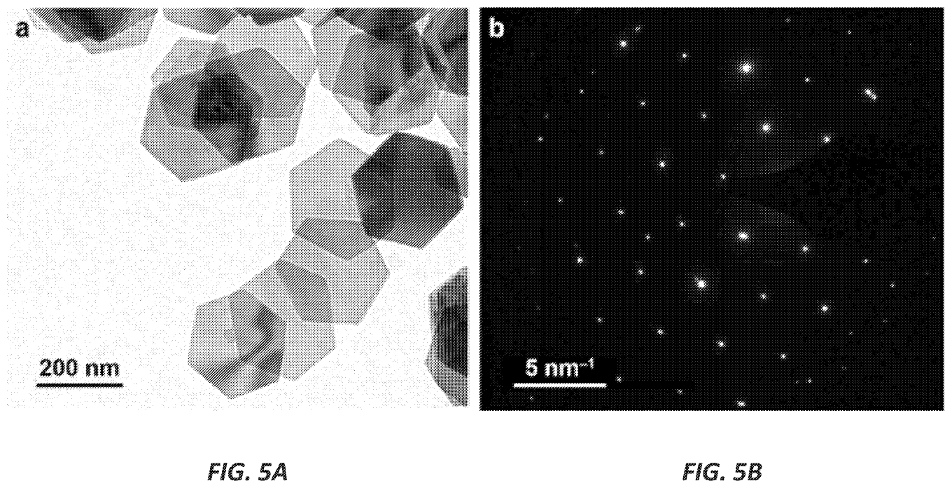

FIG. 1B illustrates an anisotropic (here, hexagonal) MND core 110. The MND core 110 may be made of magnetite (Fe₃O₄). The shape anisotropy of the MND core 110 may include, but is not limited to, oblate (e.g., disc shaped), prolate (e.g., cigar shaped), a 2-dimensional flake, a disc, and/or a 1-dimentional morphology (e.g., a nanorod). The MNDs 110 may be formed by reducing hematite nanodiscs. MND cores 110 can be synthesized via reduction of hexagonal nanodisc hematite (Fe₂O₃), starting with heating FeCl₃, sodium acetate, ethanol, and water in an autoclave reactor to generate hematite nanodiscs. FIGS. 5A-5B illustrate transmission electron microscopy (TEM) images of hexagonal hematite (Fe₂O₃) prior to reduction. Hematite nanodiscs may be produced by heating a uniform mixture of 0.273 g of FeCl₃·6H₂O, 10 mL Ethanol, and 600 μL of deionized (DI) water in a sealed Teflon- lined steel vessel at 180° C. for 18 hours. After washing the hematite nanodiscs with DI water and ethanol about 3-5 times, the dried hematite may be dispersed in about 20 mL of trioctyl-amine and 1 g of oleic acid. For the reduction of hematite to magnetite, the mixture may be transferred into a three-neck flask connected to a Schlenk line, and evacuated for 20 minutes at room temperature, and then heated to 370° C. (at a rate of about 20° C./min) in an H₂ (5%) and N₂ (95%) atmosphere for 30 minutes resulting in the MND cores 110.

Tuning the amount of water in the solution enabled aspect ratio tuning of the hematite nanodiscs, with 6-9% water producing hexagonal hematite nanodiscs. Hematite nanodiscs prepared with higher water concentrations (e.g., greater than 6-9%) may be more isotropic in shape. Hematite nanodiscs prepared with less than 6% water may have a larger diameter and/or a non-uniform morphology. The hematite nanodiscs are washed in deionized water, dried, resuspended in trioctylamine and oleic acid, and heated in a hydrogen atmosphere to be converted to magnetite. Reducing the hematite nanodiscs in a hydrogen atmosphere should yield complete conversion of nonmagnetic hematite into magnetite while maintaining the nanodiscs' anisotropic (e.g., hexagonal geometry).

The MNDs cores 110 may have a maximum lateral dimension of about 170 nm to about 550 nm, for example approximately 231+29 nm in diameter. The MNDs cores 110 may range in thickness from 10 nm to 50 nm, for example approximately 31+9 nm thick.

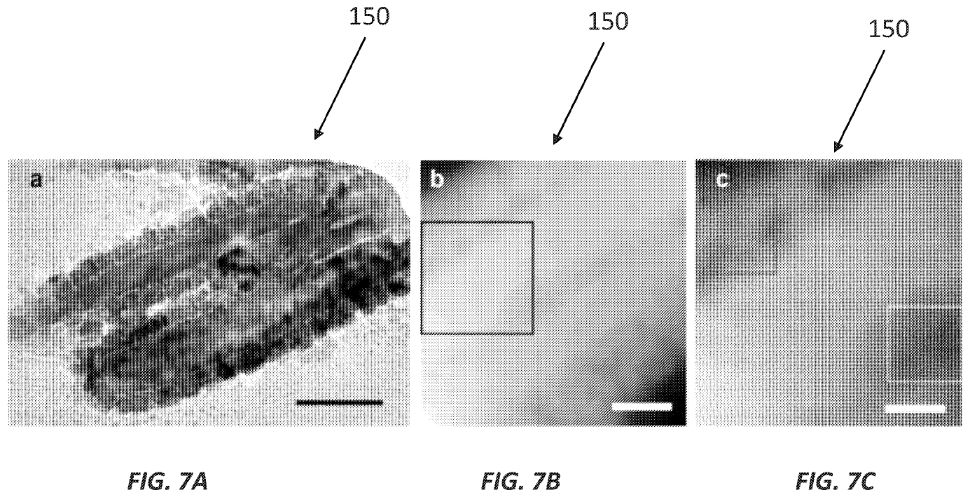

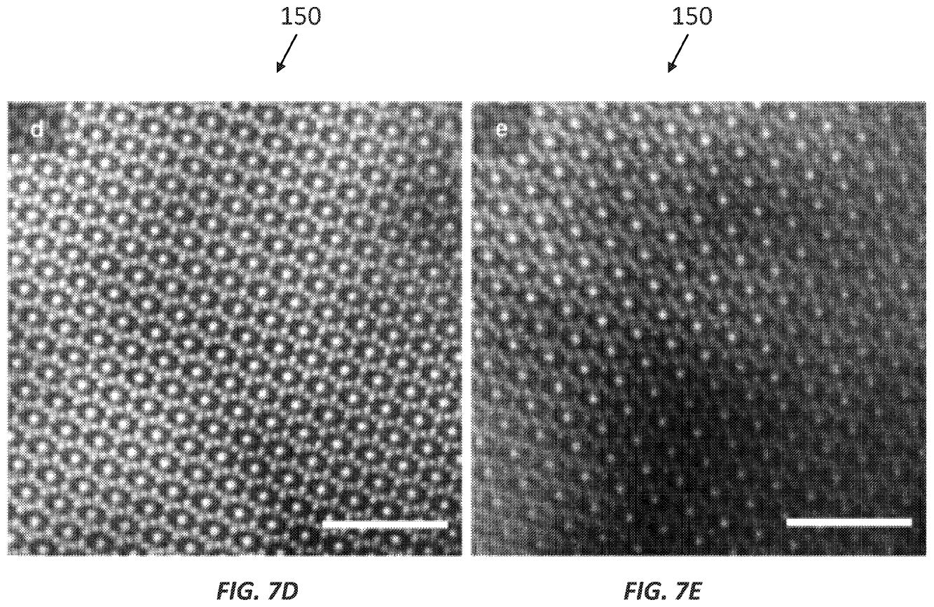

FIG. 1C illustrates a core-shell nanodisc 150 (also referred to as a core-shell nanodiscs (CFONDs) including a MND core 110 and an inner shell 120 surrounding the MND core 110. The MND cores 110 may be coated with an inner shell 120 (e.g., a first shell). A magnetostrictive inner shell 120 may add crystalline anisotropy to an anisotropic MND 110 and transforms the MND 110 into a CoFe₂O₄ on nanodisc (CFOND) 150. Preferably the inner shell 120 is a magnetostrictive inner shell 120. The inner shell 120 can include AₓFe₃₋ₓO₄, where A is one of Co, Mn, Ni, Fe, Zn, Mg, or Cu. For example, the inner shell 120 may be a CoFe₃O₄ shell. CoFe₂O₄ is particularly well suited for use as the magnetostrictive inner shell because it has a higher magnetostrictive effect (250 ppm) due to its crystalline anisotropy than Fe₃O₄ (25 ppm). (Mn, Ni, Fe, Zn, Mg, or Cu can be substituted for Co in the magnetostrictive shell.) The inner shell 120 may be applied using an organo-metallic method resulting in a CFOND 150. For example, when the inner shell 120 is a CoFe₃O₄ the resulting CFOND 150 may be a Fe₃O₄—CoFe₃O₄ CFOND 150. The CFOND 150 may be approximately 240±42 nm in diameter. FIGS. 7A-7E illustrate cross-sectional TEM images of the CFOND 150.

The CFONDs 150 may be formed by nucleation and growth of a CoFe₂O₄ layer on the surface of the MNDs 110. The magnetostrictive inner shells 120 surrounding the MND cores 110 can be formed by hetero nucleation and growth of CoFe₂O₄ using heat-up colloidal synthesis based on acetylacetone (acac) precursors. The composition of the magnetostrictive inner shell 120 may depend on the ratio of the precursors. For example, CoₓFe₃₋ₓO₄ can be synthesized with x mol of Co(acac)₂ and (3−x) mol of Fe(acac)₃, MnₓFe₃₋ₓO₄ can be synthesized with x mol of Mn(acac)₂ and (3−x) mol of Fe(acac)₃, NiₓFe₃₋ₓO₄ can be synthesized with x mol of Ni(acac)₂ and (3−x) mol of Fe(acac)₃, FeₓFe₃₋ₓO₄ can be synthesized with x mol of Fe(acac)₂ and (3−x) mol of Fe(acac)₃, ZnₓFe₃₋ₓO₄ can be synthesized with x mol of Zn(acac)₂ and (3−x) mol of Fe(acac)₃, MgₓFe₃₋ₓO₄ can be synthesized with x mol of Mg(acac)₂ and (3−x) mol of Fe(acac)₃, and CuₓFe₃₋ₓO₄ can be synthesized with x mol of Cu(acac)₂ and (3−x) mol of Fe(acac)₃.

The solution for the heat-up colloidal synthesis may include Diphenyl ether 20 mL, Oleylamine 1.97 mL, Oleic Acid 1.90 mL, and the MND cores. Using a condenser, N₂ inlet, and magnet stirring at 800 rpm, the heat-up colloidal synthesis process is as follows: approximately 120 mg of MNDs cores 110 may be dispersed uniformly in a precursor solution of 20 mL diphenyl ether, 1.90 mL oleic acid, 1.97 mL oleylamine, 257 mg cobalt acetylacetonate (Co(acac)₂), and 706 mg iron acetyl acetonate (Fe(acac)₃). A three-neck flask including the solution of MND cores 110 and the shell precursors may be connected to a Schlenk line. The solution may be evacuated and then heated to 100° C. (at a rate of about 7° C./min) for 30 minutes in N₂ atmosphere while magnetically stirring at 400 rpm. After closing the N₂ line, the temperature may be increased to 200° C. (at a rate of about 7° C./min) and maintained for 30 minutes, and then increased to 230° C. (at a rate of about 7° C./min) and maintained for 30 minutes to 2 hours. The solution may be cooled to room temperature (approximately 30 minutes), and the resulting CFONDs 150 may be washed with ethanol and n-hexane and subjected to centrifugation at 8000 rpm for 8 minutes; the washing process may be repeated 2-3 times. The thickness of the inner shell 120 (e.g., the CoFe₂O₄ layer) may be controlled by repeating the organometallic synthesis and washing steps described above. For example, a 5 nm CoFe₂O₄ layer may be prepared by repeating the synthesis three times.

In the colloidal synthesis process, the speed of the temperature increase may vary depending on the type of temperature controller. Additionally, stirring for 30 minutes at 200° C. under N₂ flow may be optional in the synthesis process. Furthermore, the time at 230° C. may vary from 30 minutes to 2 hours depending on the thickness of the inner shell 120.

FIG. 1K illustrates a cross-section of the CFOND 150 including a MND core 110 and inner shell 120 surrounding the MND core 110. As shown in FIG. 1K, the inner shell 120 may completely surround the MND core 110. The inner shell 120 may include a plurality of layers.

FIGS. 1L and 1M illustrate inner shells 120 and 120′, each with a plurality of layers 121. The thickness of the magnetic inner shell 120 can be controlled by repeating the heat-up colloidal synthesis process described immediately above, with each time through the process yielding another layer 121 of AₓFe₃₋ₓO₄. FIG. 1L illustrates an inner shell 120 with multiple layers 121 (e.g., multiple layers of AₓFe₃₋ₓO₄). For example, repeating the heat-up colloidal synthesis process three times yields a (inverse) spinel-crystal structured ferrite inner shell 120 that is about 5 nm thick (e.g., 1.67 nm per layer). The inner shell 120 may be about 3 nm to about 10 nm thick. There is no upper limit in the thickness of the inner shell 120, though thicker inner shells 120 may increase the magnetic crystalline anisotropy and decrease the shape anisotropy. Increasing the thickness of the inner shell 120 beyond 0 nm may decrease the shape anisotropy of the inner shell 120. The magnetic crystalline anisotropy may increase according to the enhancement of the mass of the materials with the larger intrinsic magnetic crystalline anisotropy.

If desired, the composition of the inner shell's layers 121 can change, e.g., to have a stoichiometry that varies with radius from the center of the MND core 110. For example, the inner shell 120′ in FIG. 1M includes a first layer 121a with a first stoichiometry and a second layer 121b with a second stoichiometry that is different from the first stoichiometry. Alternatively, the composition of the inner shell's layers 121 may also change to have different compositions. For example, the inner shell 120″ in FIG. 1N includes a first layer 121c with a first composition (e.g., AₓFe₃₋ₓO₄ (A=Co, Mn, Ni, Fe, Zn, Mg, or Cu)) and a second layer 121d with a second composition (e.g., AₓFe₃₋ₓO₄ (where A=Co, Mn, Ni, Fe, Zn, Mg, or Cu and)) that is different from the first composition (e.g., where A in the first layer 121c and A in the second layer 121d are different).

Further examples of the preparation and use of CFONDs may be found in U.S. application Ser. No. 18/635,476, which is hereby incorporated by reference in its entirety.

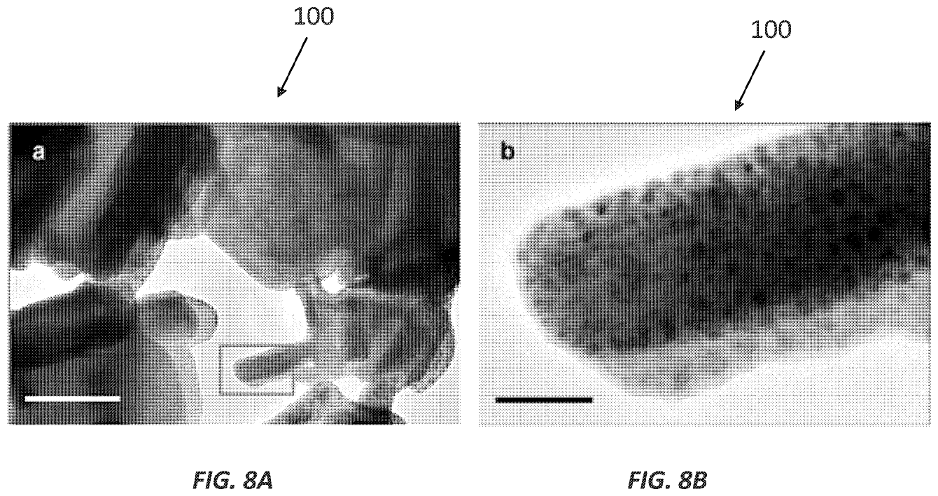

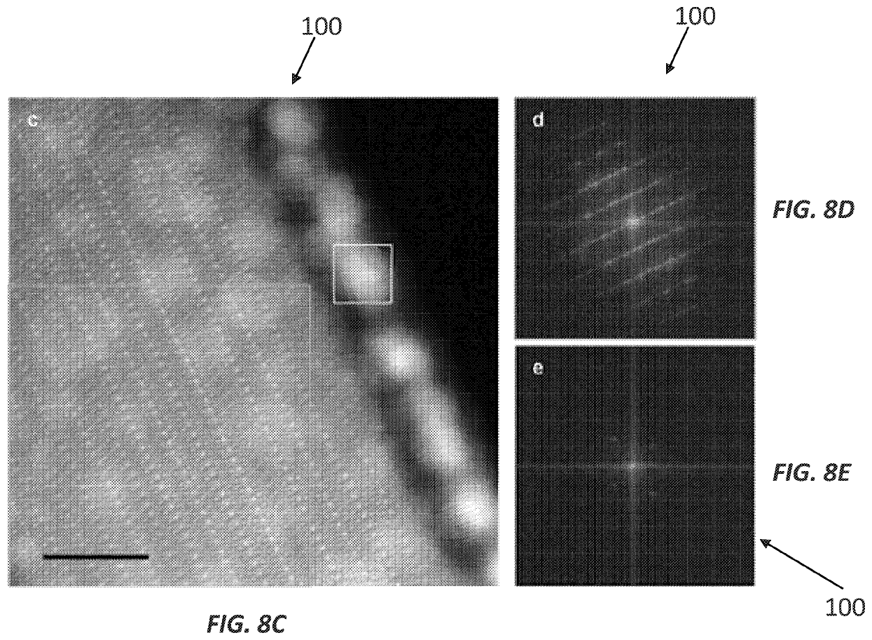

FIG. 1D illustrates core-double shell nanodiscs (also referred to as magnetoelectric nanodiscs (MEND) 100). To form a MEND 100 an outer shell 130 (e.g., a second shell) may be grown on the CFOND 150. Preferably the outer shell 130 is a piezoelectric shell. The outer shell 130 may be made of any suitable piezoelectric material. For example, the outer shell 130 may include, but is not limited to, barium titanate (BaTiO₃), lead zirconate titanate (PZT) (Pb[ZrₓTi₁₋ₓ]O₃ (0≤x≤1)), lead titanate (PbTiO₃), or barium-calcium zirconium titanate (BCZT). The outer shell 130 may be grown onto the CFONDs 150 via a combination of sol-gel synthesis and calcination resulting in a MEND 100. For example, the MEND 100 may be a core-double shell Fe₃O₄—CoFe₂O₄—BaTiO₃ MEND. The MEND 100 may be approximately 250+41 nm in diameter. FIGS. 1D, 6, 8A-8E illustrate MENDs 100. FIGS. 8A-8E illustrate cross sectional TEM images of MENDs 100.

The MEND 100 may be prepared by formation of the outer shell 130 (e.g., a BaTiO₃ shell) on the surface of the CFOND 150 via a sol-gel method. A mixture comprising 16 mg of CFONDs 150 dispersed in n-hexane, 30 mL of DI water, 6 mL of ethanol, and 2 g of poly(vinylpyrrolidone) may be sonicated for 20 minutes, to lead to a segregation of the oil phase. The oil phase and other insoluble solids may then be removed with a spatula. The hydrophilic CFOND 150 dispersions may then be transferred to a three-neck flask connected to a Schlenk line, and then dried in vacuum at 80° C. until an amber-colored gel was formed on the bottom of the flask. The gel may then be re-dispersed in a BaTiO₃ shell precursor solution, which was prepared by mixing 0.5 g citric acid, 24 μL titanium isopropoxide dissolved in 15 mL of ethanol and 0.1 g citric acid, and 0.0158 g barium carbonate dissolved in DI water. The solution of CFONDs 150 and BaTiO₃ precursors may then be moved to a three-neck flask connected to a vacuum line and kept at 80° C. for 12-14 hours. The powders may then be moved to a clean ceramic container and heated at 600° C. for approximately 2 hours, 700° C. for approximately 2 hours, then 800° C. for approximately 1 hour, sequentially. To prevent breaking the outer shell 130, the furnace door may be kept closed until the temperature slowly cooled down to room temperature. The MENDs 100 may then be dispersed in Tyrode and/or phosphate buffered saline (PBS) for in-vitro and in-vivo experiments described below.

FIG. 1E illustrates the electrochemical measurement apparatus that employs surface charge variation of MENDs 100 in response to an applied MF that was used to determine the magnetoelectric coefficient (α_{ME}) of the MENDS 100. The addition of the inner shell 120 (e.g., a CoFe₂O₄ shell) may allow for an increased magnetocrystalline anisotropy and magnetostrictive response compared to the MND core 110 (e.g., Fe₃O₄) alone. In addition to a greater bulk magnetostriction coefficient (250 ppm for CoFe₂O₄ vs. 25 ppm for Fe₃O₄), the interfacial spring exchange effect may increase the anisotropy of the resulting CFOND 150 and thus the magnetostrictive response of the CFOND. In addition, the direct interfacing of materials with different magnetostrictive constants may yield an enhanced magnetically-induced strain. The illustration in FIG. 1E was used to numerically predict the magnetoelectric coupling coefficient (α_{ME}) of the MENDs 100.

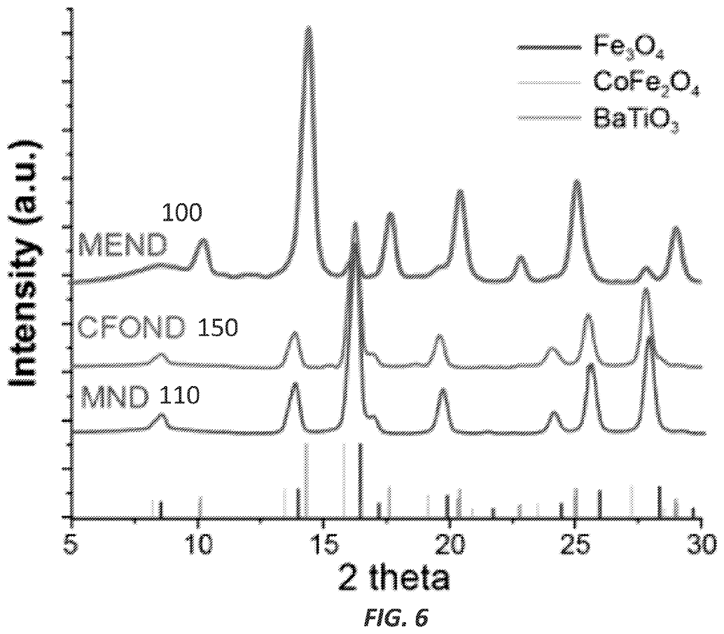

FIG. 6 illustrates X-ray diffraction spectra for MND core 110, CFOND 150, and MEND 100.

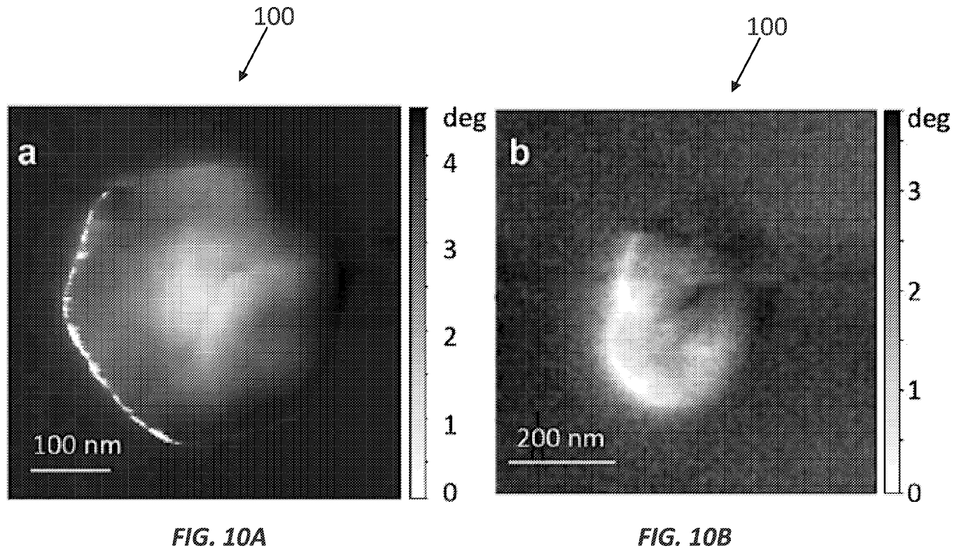

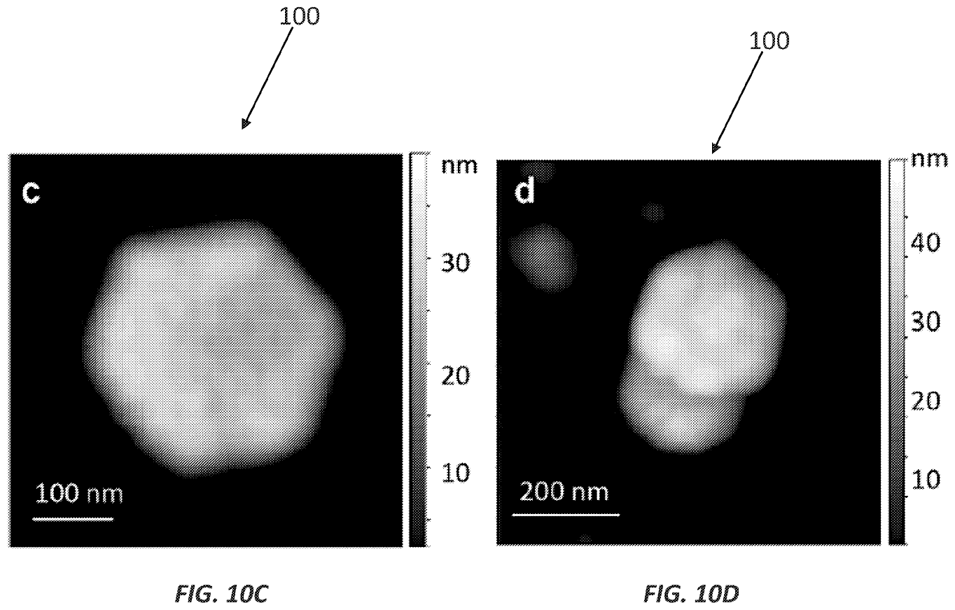

Using micromagnetic simulation to quantify the effective deformation of nanoparticles due to magnetostriction, CFONDs 150 were compared with spherical Fe₃O₄—CoFe₂O₄ core-shell nanoparticles (also referred to as isotropic MENPs or MENPs 140) of the same volume and volumetric ratio of CoFe₂O₄ to Fe₃O₄. The increase in shape anisotropy in CFONDs 150 resulted in approximately three orders of magnitude enhancement in effective deformation over the isotropic MENPs 140 (0.83026 ppm for CFOND 150 vs. 0.000808 ppm for isotropic MENPs 140) (see FIG. 1F). For the accuracy of the micromagnetic simulation, the remnant state of CFONDs 150 was found to be either a magnetic vortex or an in-plane macrospin as observed with magnetic force microscopy (see FIGS. 10A-10D).

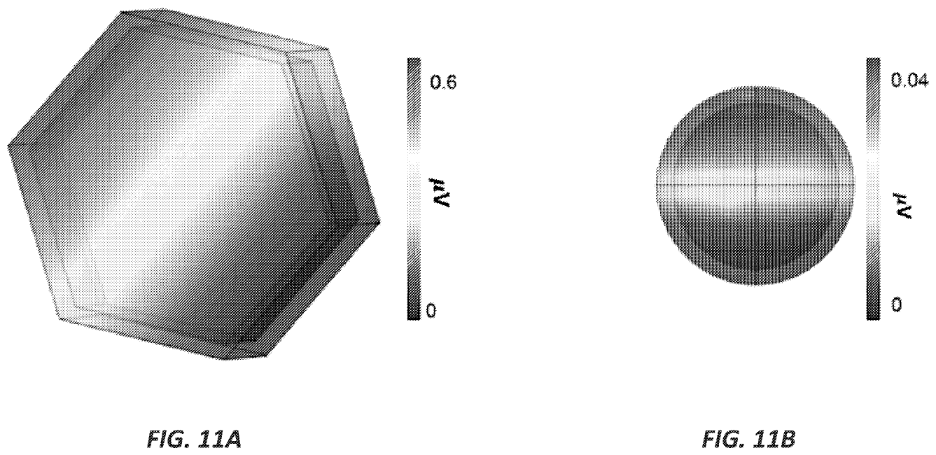

The interaction between Fe₃O₄ and CoFe₂O₄ in CFONDs 150 yields almost 40 times enhancement in effective deformation as compared to Fe₃O₄ MNDs cores 110 with the same volume (0.01898 ppm for the MND core 110; see FIG. 1F) due to the interfacial spring exchange coupling and surface magnetostriction. The results of the micromagnetic simulation were then introduced into a finite element model of the piezoelectric response of the BaTiO₃ shell, to demonstrate the scaling of the magnetoelectric coefficient (α_{ME}) with the magnetostriction effect. The numerical simulations also predicted an improved ME coupling in MENDs 100 as compared to MNDs 110 or isotropic MENPs 140 of the same volume (see FIGS. 1G, 11A, and 11B). These simulations were then experimentally confirmed.

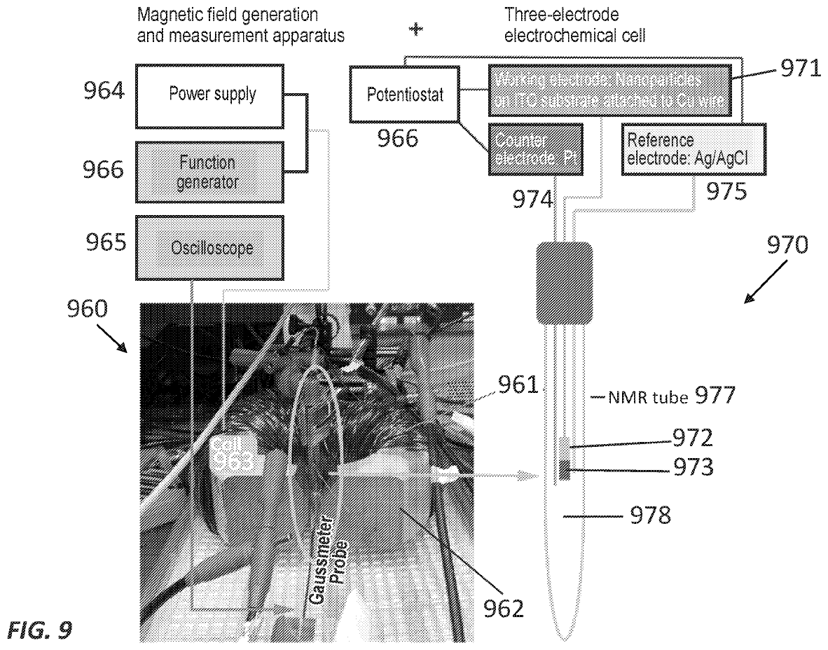

FIG. 9 illustrates the experimental α_{ME} measurement apparatus 960. To experimentally evaluate the α_{ME} of the MENDs 100, electrochemical means were leveraged to determine ME coupling from nanoparticles dispersed in an electrolyte. This approach was to MEND films. Tyrode's solution was used an electrolyte and a three-electrode electrochemical cell 970 was constructed. The electrochemical cell 970 may include a working electrode 971 made up of a MEND layer 972 on a conductive indium tin oxide substrate 973 with a counter electrode 974 (e.g., a platinum (Pt) electrode) and a reference electrode 975 (e.g., a silver and/or silver chloride (Ag/AgCl) electrode) (see FIGS. 1E and 9). The electrochemical cell 970 may be operably connected to a potentiostat 976 to measure and control the potential difference between the working electrode 971, the counter electrode 974, and the reference electrode 975 The electrochemical cell 970 may be contained in a nuclear magnetic resonance (NMR)glass tube 977 (e.g., about 8 mm diameter). The glass tube 967 may be filled with an electrolyte 978 (e.g., Tyrode's solution, phosphate buffered saline (PBS), Tris-acetate-EDTA (TAE)) and the electrochemical cell 970 may be immersed in the Tyrode's solution.

The measurement apparatus 960 may include a copper magnet wire 961 (e.g., TEMCo 14 AWG)wound around a horseshoe-shaped magnetic core 962 with approximately a 0.5-inch gap to form a coil 963. The coil 963 may generate a magnetic field. The coil 963 may be connected to a power supply 964 (e.g., a CROWN DC-300A Series II) and signal generator or oscilloscope 965 (e.g., a PICOSCOPE 2204A) to generate a compound magnetic field combining an offset magnetic field (OMF, 0-320 mT) with an alternating magnetic field (AMF, 0-1 kHz, 0-14 mT). The measurement apparatus 960 may also include a function generator 966. The glass tube 977 and electrochemical cell 970 may then be placed into a gap of in the coil 963 as shown in FIG. 9.

To observe ME behavior, the nanoparticles of the MEND layer 972 were simultaneously exposed to a constant offset magnetic field (OMF), which magnetized them to near saturation where the change in slope of the magnetization as a function of field amplitude is maximized, and an alternating magnetic field (AMF) which drove magnetization change to induce magnetostriction. The potential change under the different amplitudes and frequencies of MFs was recorded at a constant current of approximately 10 nA by a potentiostat 976, allowing the electric double layer capacitance to be measured, while avoiding faradaic reactions. The potential changes were normalized to the AMF amplitude and the MEND layer 972 thickness to obtain the α_{ME} values.

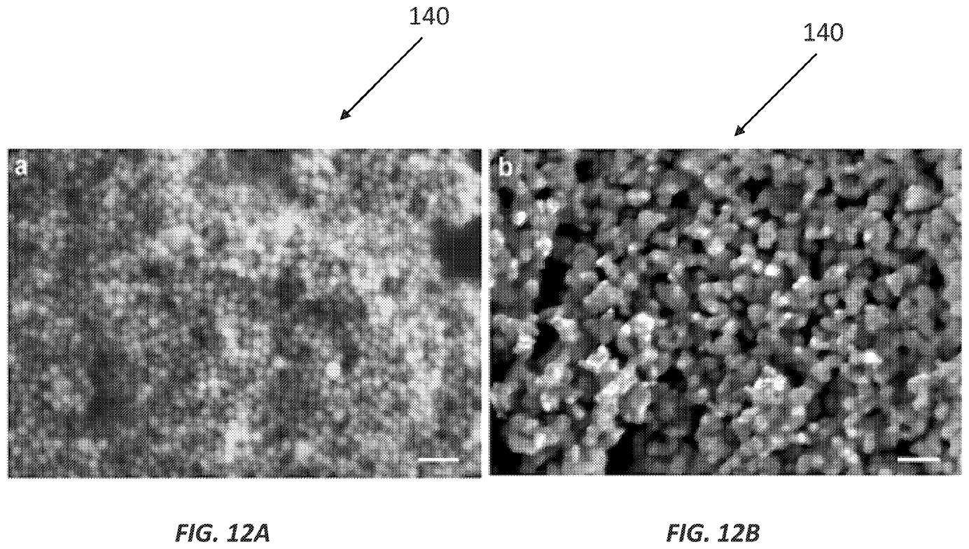

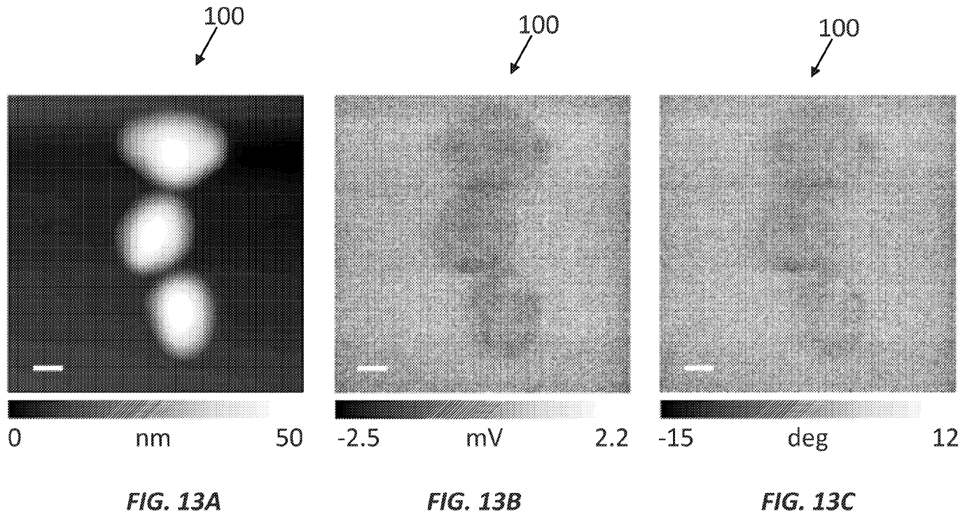

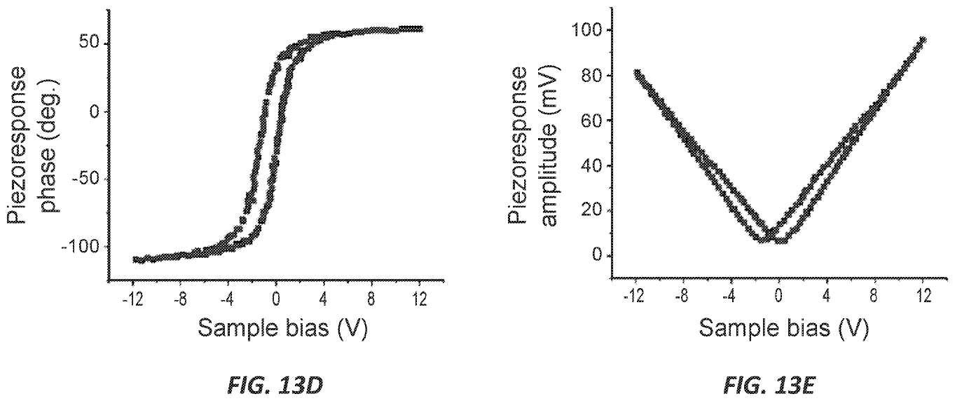

The α_{ME} of MENDs 100 was measured to be approximately 150 mV mT⁻¹ cm⁻¹, which is about 4 times higher than the peak value for isotropic MENPs 140 (as shown in FIGS. 1H, 12A and 12B). The discrepancy between the simulated >1000 times increase in the strain response (fee FIG. 1F) and the experimentally observed 4 times increase in α_{ME} may be attributed to the compensation of the electric polarization in a multi-domain piezoelectric outer shell 130 (see FIGS. 13A-13E) and the lattice mismatch between the inner shell 120 (e.g., cubic CoFe₂O₄) and the outer shell 130 (e.g., tetragonal BaTiO₃), which may result in strain relaxation at the interface between the inner shell 120 and the outer shell 130. The lattice mismatch between the inner shell 120 and the outer shell 130 may vary based on the composition of the inner shell 120 and the outer shell 130. A larger mismatch between the inner shell 120 and the outer shell 130 may result in a larger strain transportation from the inner shell 120 to the outer shell 130 due to a harder-to-form epitaxial interface.

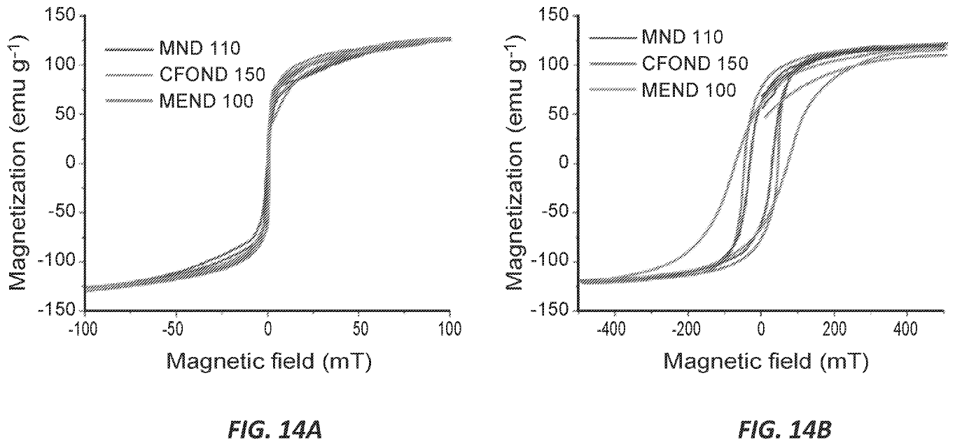

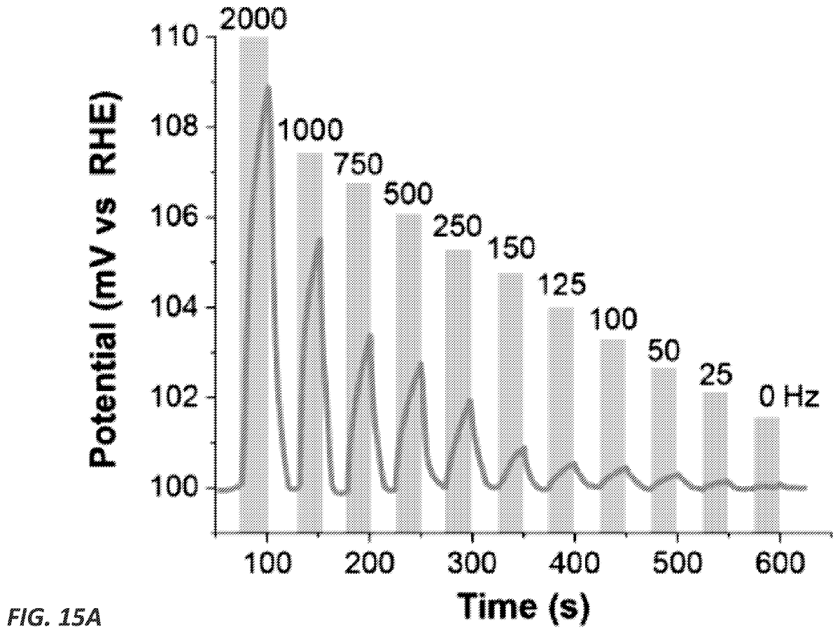

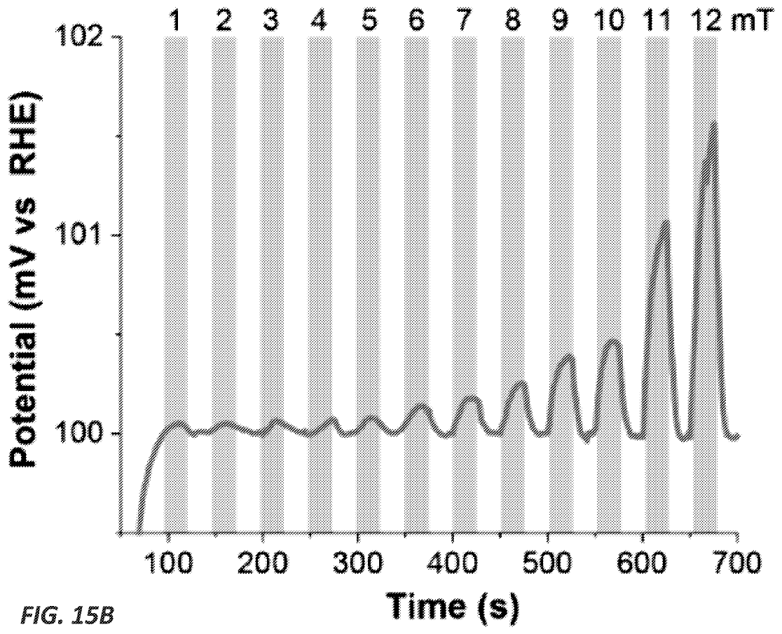

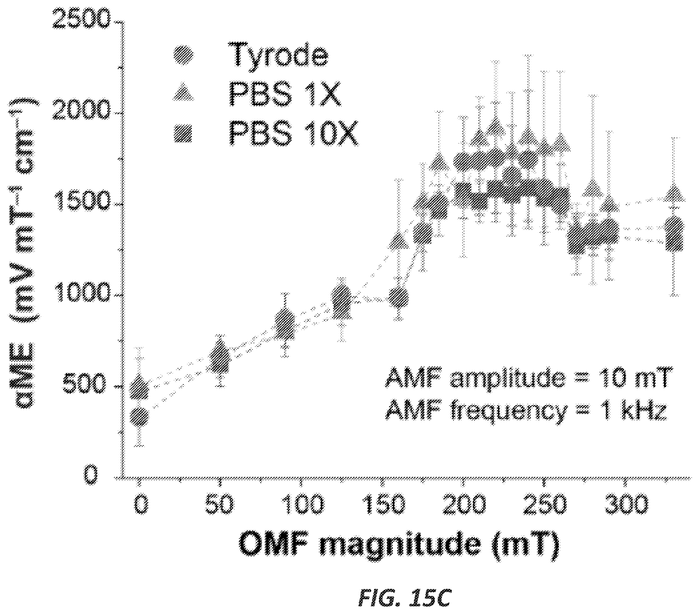

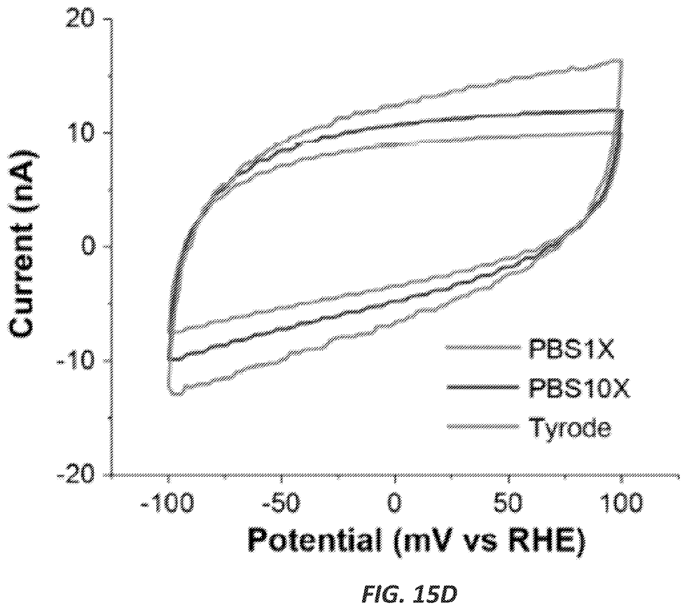

The ME coefficient peaked at approximately 220 mT of OMF, which is near the saturation field of the MENDs 100 recorded via vibrating sample magnetometry shown in FIGS. 14A and 14B. Fixing the amplitude of the OMF at 220 mT, the α_{ME} was then found to increase with increasing AMF frequency (see FIGS. 11 and 15A), which may be expected for frequencies below the piezoelectric resonance in the MHz range. The AC magnetic field amplitude did not have an impact on the α_{ME} (see FIGS. 1J and 15B). The ME measurement approach was independent of the identity or concentration of ions in the electrolyte 978, as the α_{ME} was found to be the same in normal and 10-times concentrated phosphate buffered saline (PBS) as in Tyrode's solution (see FIG. 15C) despite the differences in voltammograms expected from the variation in double-layer capacitance (see FIG. 15D).

### MEND-Mediated Neuromodulation In Vitro

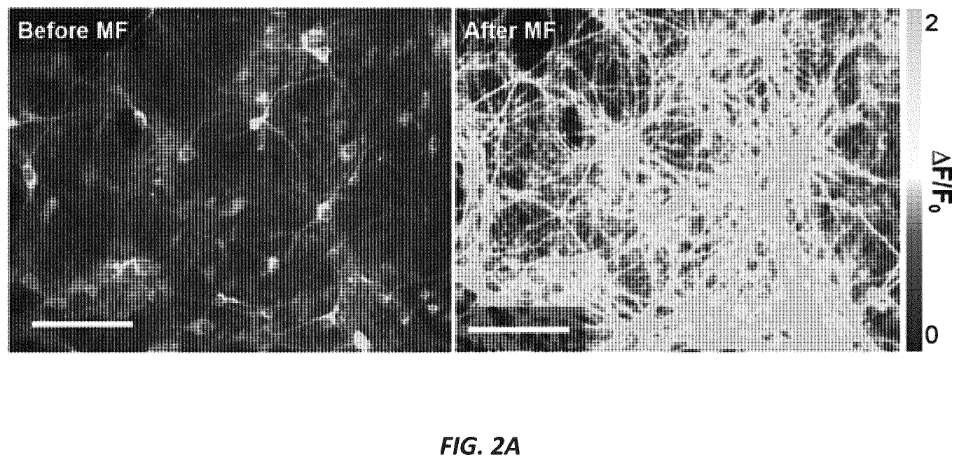

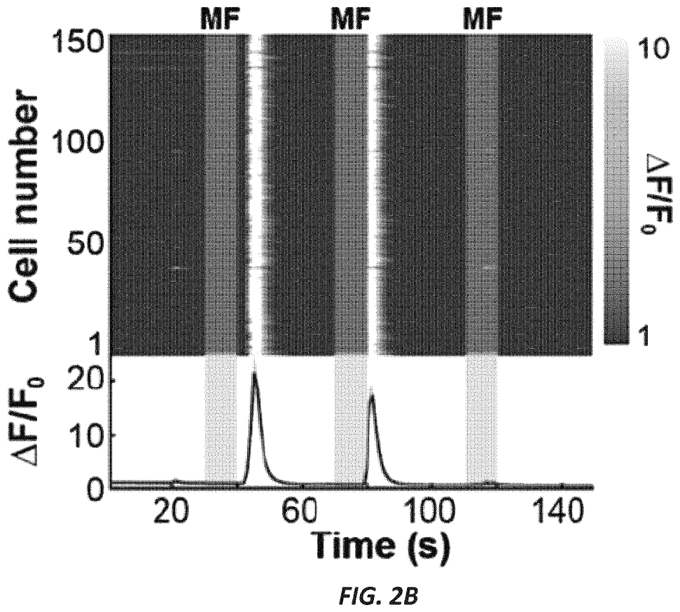

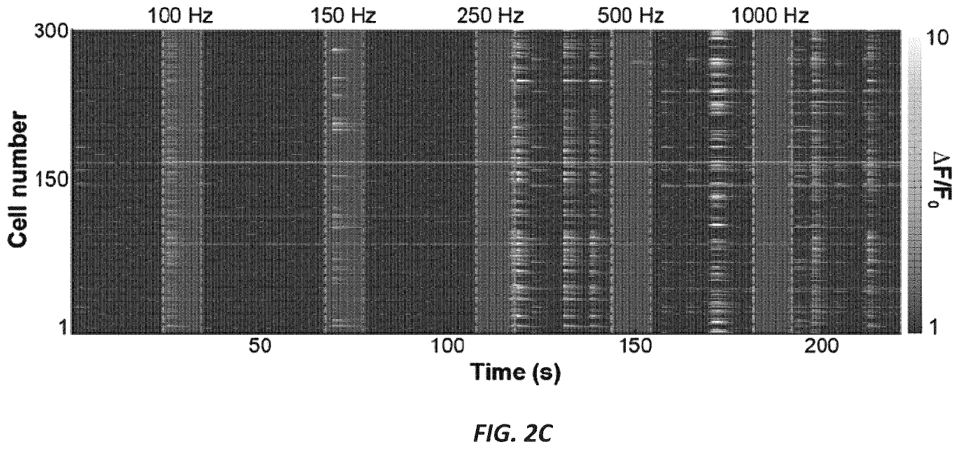

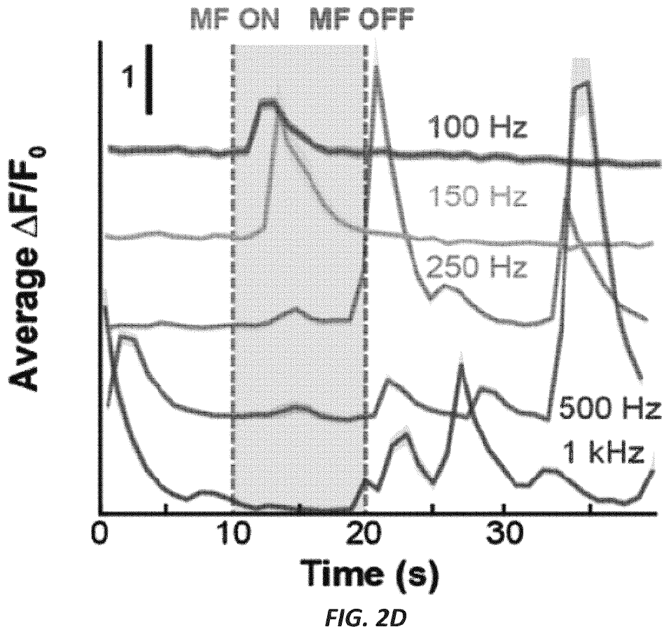

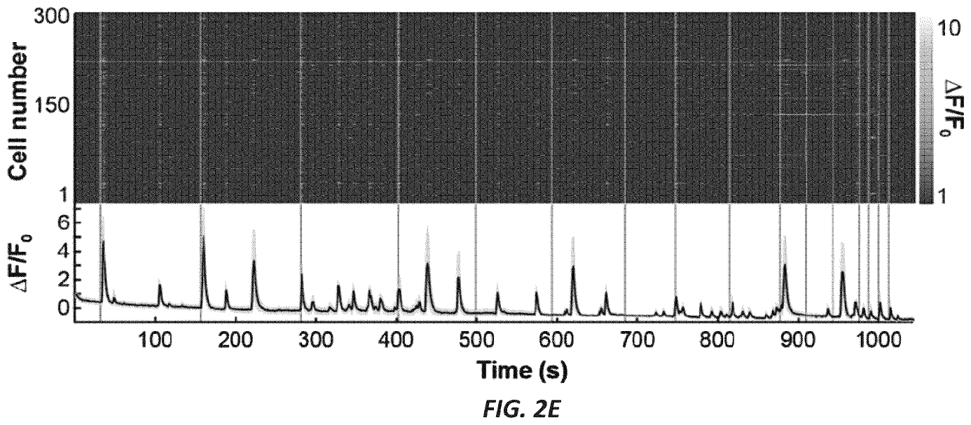

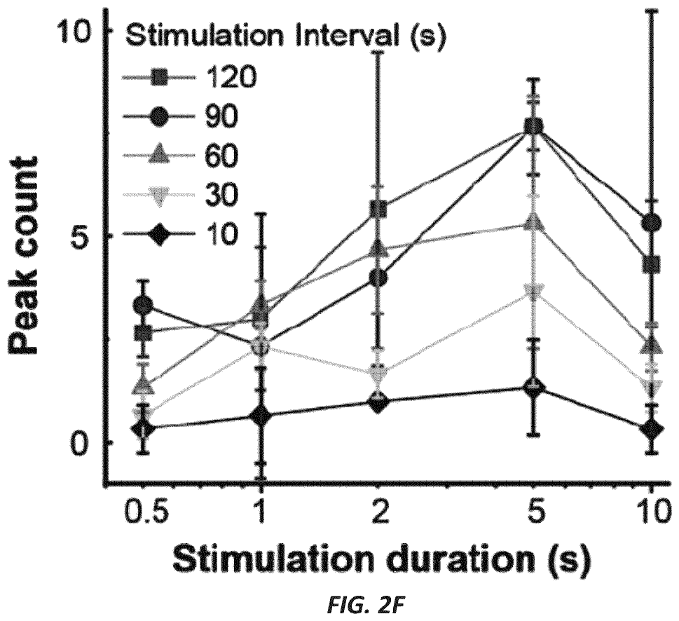

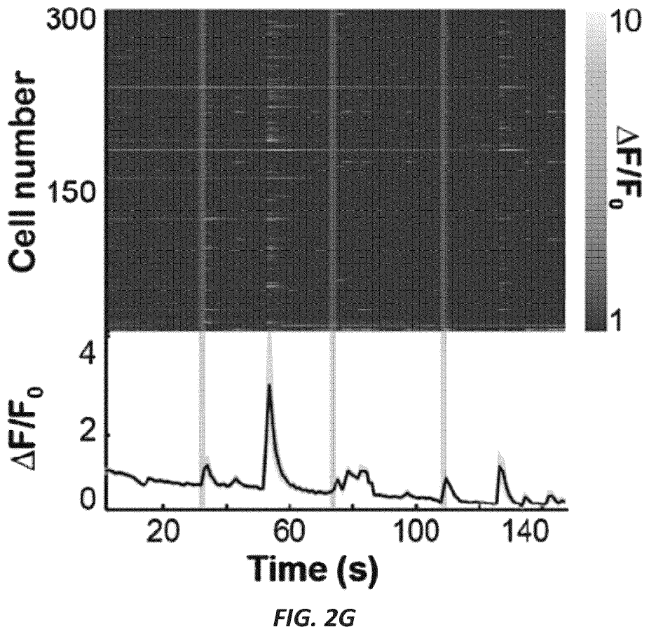

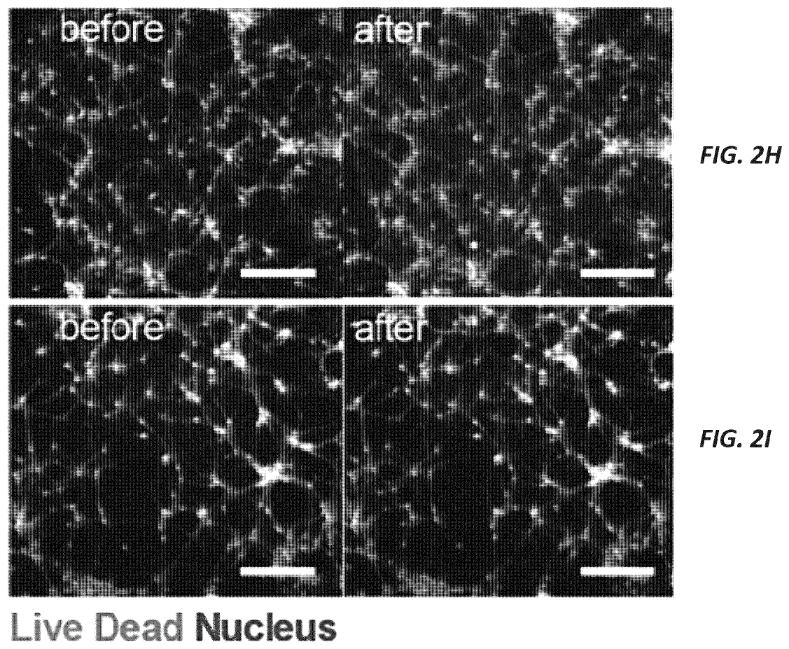

The efficacy of ME modulation mediated by MENDs 100 in cultured primary hippocampal neurons virally transduced to express a fluorescent calcium (Ca²⁺) indicator GCaMP6s was evaluated as a proxy for neuronal activity. ME neuromodulation with MENDs 100 was expected to be most effective when the particles were in direct contact with neuronal membranes (e.g., membrane 106). Consequently, all experiments in vitro were conducted following a 1-hour incubation period to allow the MENDs 100 to precipitate onto the cells (e.g., cell 104). The MEND 100 density on the cell membranes was measured by quantifying their mass on each sample and calculating the area occupied by the neuronal network. Robust increases in GCaMP6s fluorescence were observed in neurons upon exposure to 10 seconds of combined 220 mT OMF and 10 mT, 1 kHz AMF in the presence of MENDs delivered at a density of 1 μg/mm² as shown in FIGS. 2A and 2B. This excitation could be repeated two times. However, a decreased response was observed following the third MF epoch potentially due to excitotoxicity. The MEND particle amount, duration and/or interval of the magnetic field epochs, and/or the frequency and/or amplitude of the magnetic field may be modified to achieve an optimal stimulation.

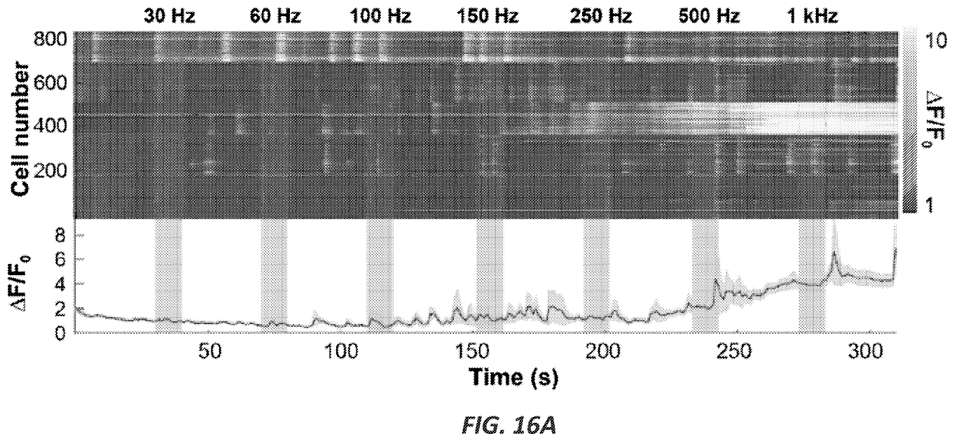

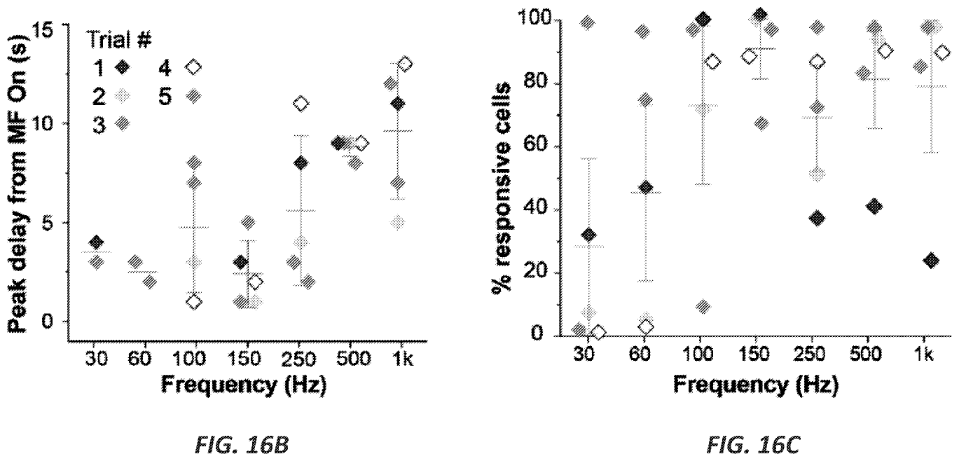

Although α_{ME} scales with the AMF frequency, resulting in larger fluctuations of the membrane potential, high frequency (e.g., >150 Hz) stimulation may yield a neuronal conduction block. This suggests the existence of a preferred frequency range for neuromodulation mediated by MENDs 100. Indeed, for AMF frequencies >150 Hz, the neurons appeared silenced during MF (220 mT OMF and 10 mT AMF) exposure and rebound activity was observed upon MF removal as shown in FIGS. 2C, 2D, and 16A-16C (. In contrast, at frequencies of 100 Hz and 150 Hz, GCaMP6s fluorescence transients emerged during the MF as shown in FIGS. 16A-16C. Furthermore, at frequencies <100 Hz, probability of observing GCaMP6s transients in response to MF stimuli was also reduced as shown in 16A-16C, which may in part be due to the reduced α_{ME} (see FIG. 1I). Therefore, approximately 150 Hz may be a preferred frequency range for neuromodulation mediated by MENDs 100. A frequency of approximately 150 Hz was used the AMF frequency for the following experiments in vitro and in vivo. Frequencies lower than about 150 Hz may result in excitation of the cells and frequencies higher than about 150 Hz may result in inhibition of the cells.

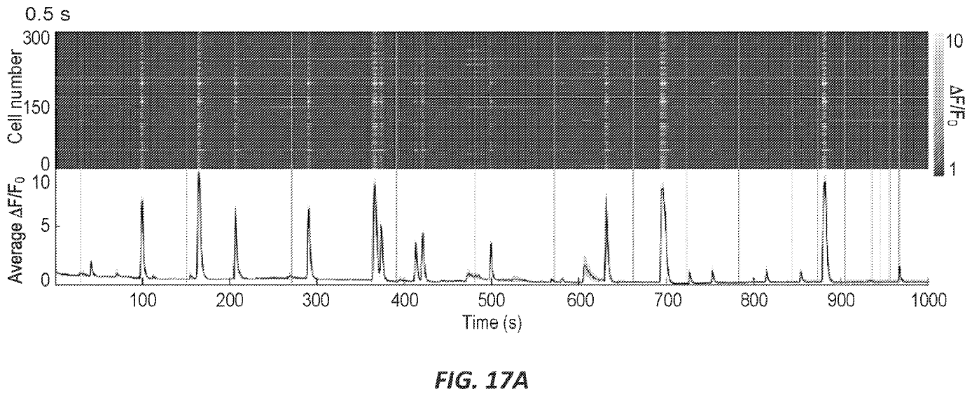

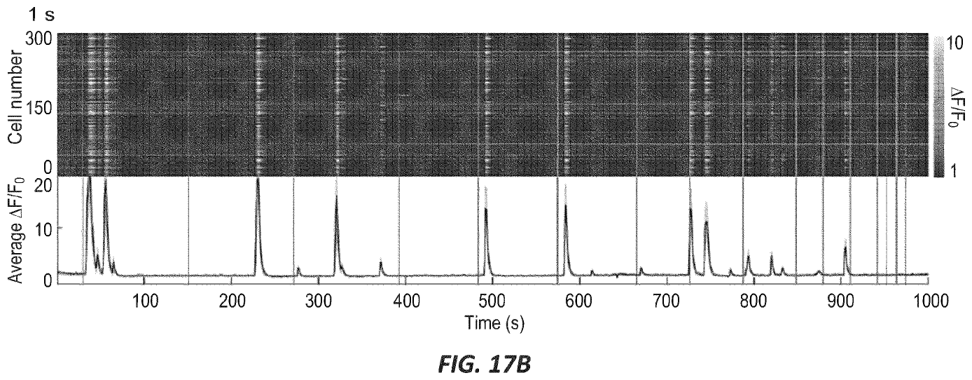

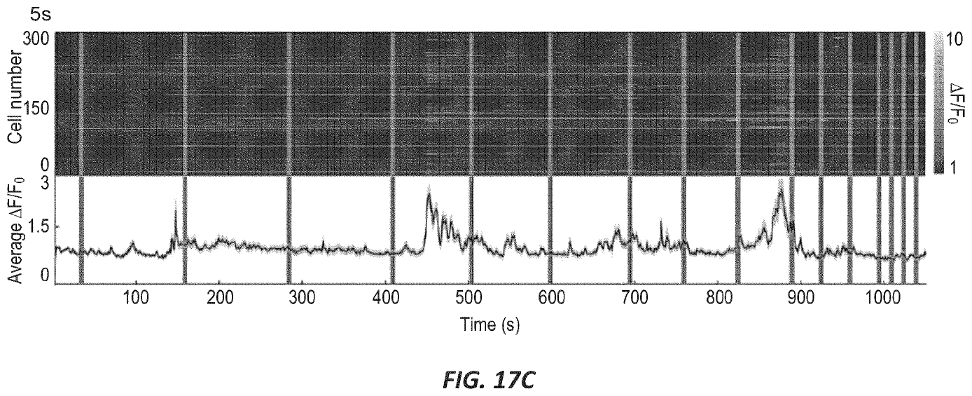

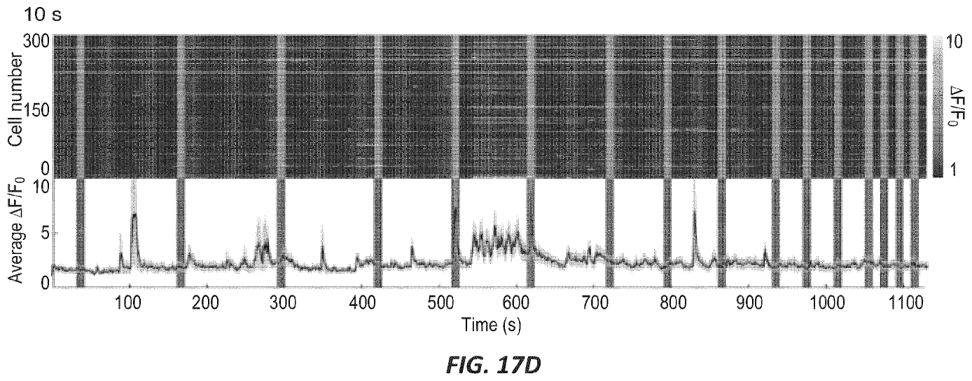

The neuronal excitation as a function of stimulation duration and interval between field epochs was further explored. The number and latency of GCaMP6s fluorescence transients for field epochs (e.g., 220 mT OMF; 10 mT, 150 Hz AMF) ranging between 0.5-10 seconds separated by rest epochs between 10-120 seconds was quantified. Stimulation epochs longer than 2 seconds produced a greater number of GCaMP6s transients than 1 second and 0.5 second stimulation epochs regardless of the rest epoch length as shown in FIGS. 2E, 2F, and 17A-17D. However, unlike short stimulation epochs that produced approximately 2.0±1.6 GCaMP6s fluorescence transients per epoch, longer stimulation epochs yielded unsynchronized responses (e.g., approximately 3.9±3.09 GCaMP6s transients as shown in FIG. 2F). A decrease in the number of magnetically evoked GCaMP6s transients for stimulation epochs separated by ≤90 seconds was observed. This is consistent with activity suppression observed in mature hippocampal cultures between bursts occurring spontaneously every 25-83 seconds.

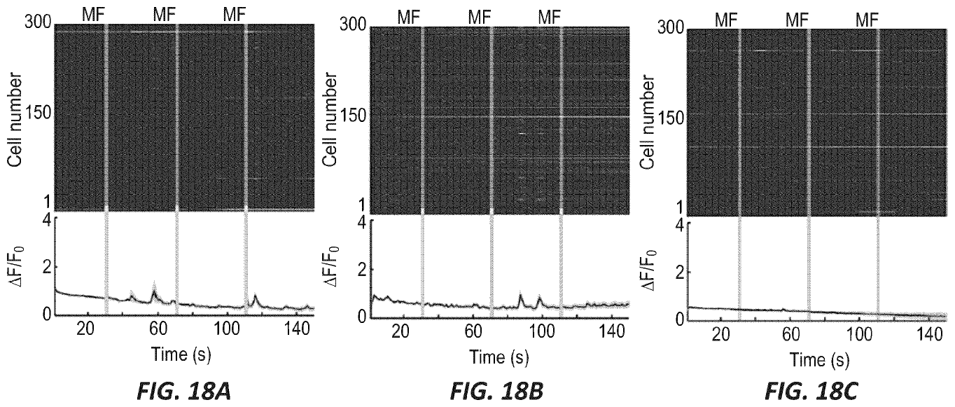

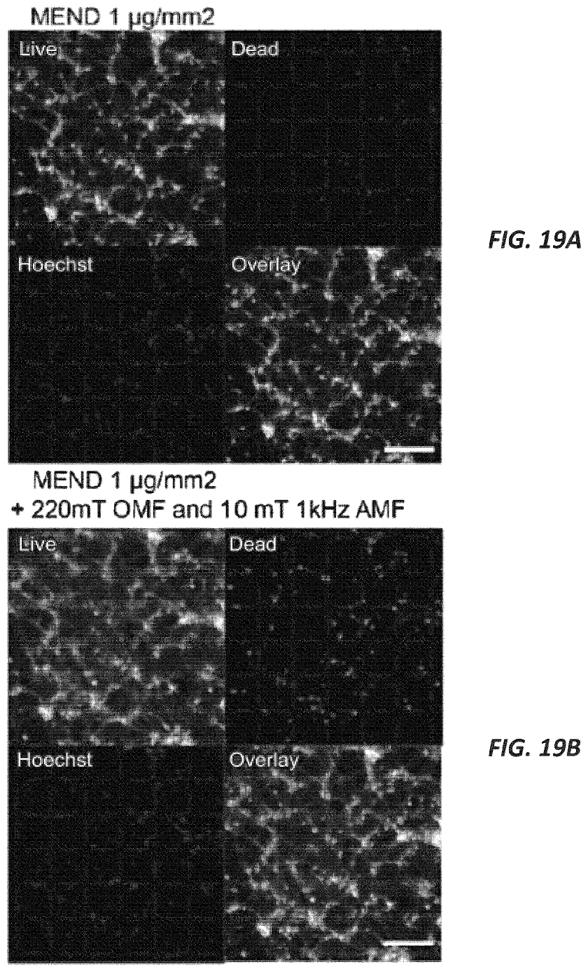

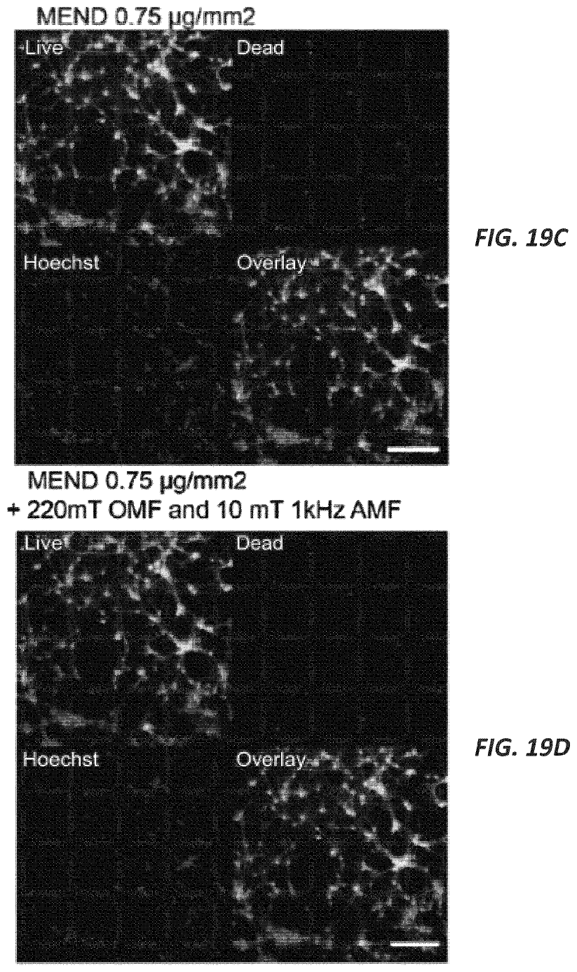

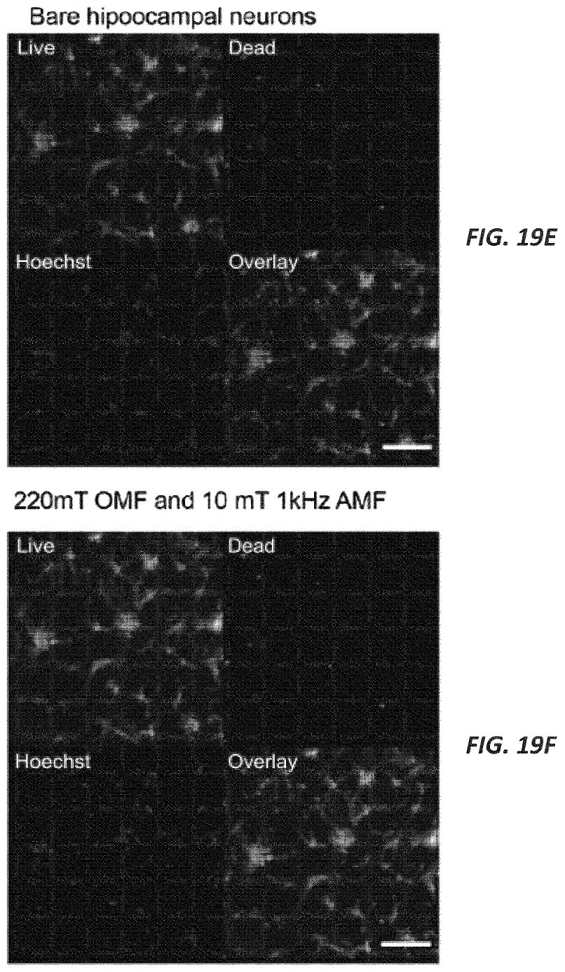

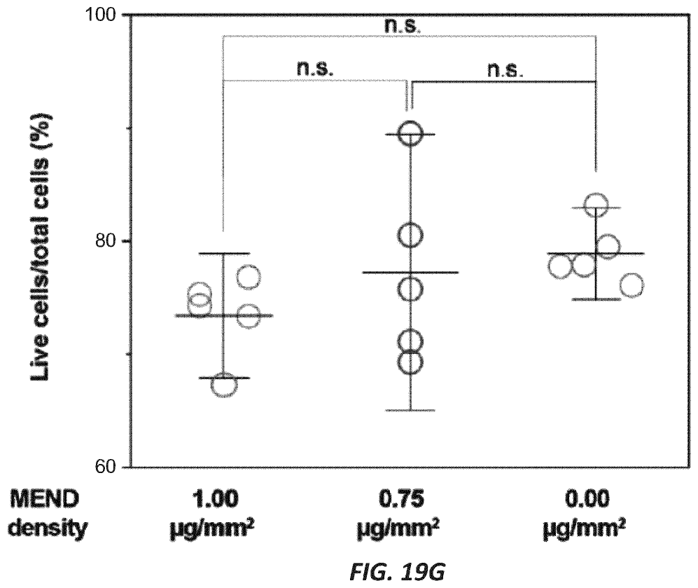

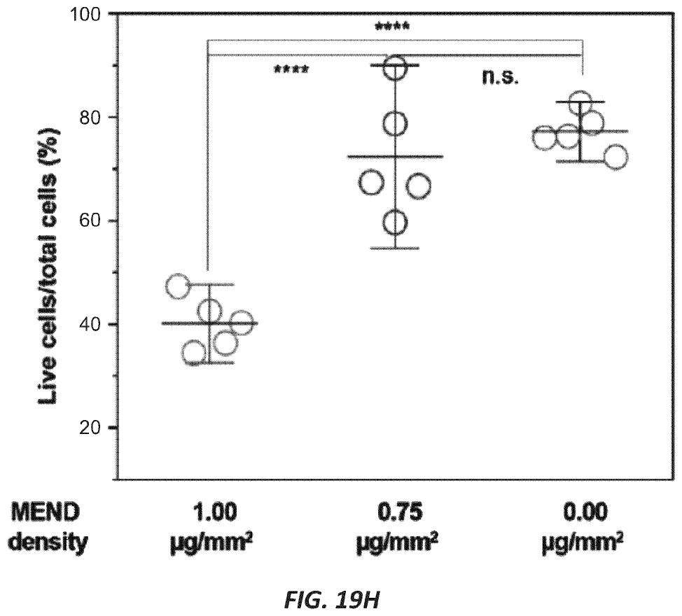

As expected, the extent of neuronal response to the MF stimuli (e.g., 220 mT OMF; 10 mT, 150 Hz AMF) scaled with the MEND density. For example, at a MEND density of approximately 1 μg/mm², approximately 73.9+15.2% of neurons were responsive to MF; at a MEND density of approximately 0.75 μg/mm², approximately 74.1±23.6% of neurons were responsive to MF; at a MEND density of approximately 0.5 μg/mm², approximately 46.3±27.6% of neurons were responsive to MF; and at a MEND density of approximately 0.25 μg/mm², approximately 27.6±23.3% of neurons were responsive to MF as shown in FIGS. 2G, 18A, and 18B. In the absence of MENDs 100, no significant response to identical magnetic field conditions was observed as illustrated in FIG. 18C. The excitotoxity of the ME modulation was similarly evaluated at different MEND 100 densities. The viability of neurons prior to ME modulation was not affected by the presence of MENDs 100 alone, at 1 μg/mm², or 0.75 μg/mm² densities as compared to cultures without MENDs 100 as illustrated in FIGS. 19A-19H. At a MEND 100 density of approximately 1 μg/mm², three 10 second repetitions of combined MF stimulus led to a decrease in neuronal viability in vitro from approximately 73.4±3.65% to approximately 40.1±5.09%, while no significant decrease in viability was observed at a MEND 100 density of approximately 0.75 μg/mm² as shown in FIGS. 2H-2J and 19A-19H. The observed excitotoxicity of ME neuromodulation at a MEND density of approximately 1 μg/mm² may explain the reduced cell responsiveness to the third stimulation epoch at a MEND density of approximately 1 μg/mm² (see FIGS. 2A and 2B). Additionally, these findings may support the use of lower MEND 100 concentrations and AMF frequencies to achieve repeatable and safe ME neuromodulation. For example, the MEND concentration may range from about 0.5 mg/ml to about 1.5 mg/ml. The AMF frequency may be about 150 Hz.

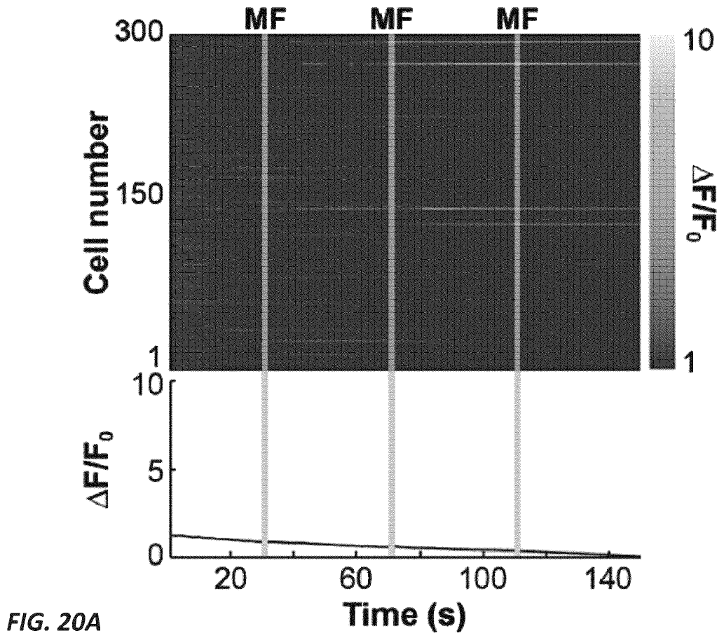

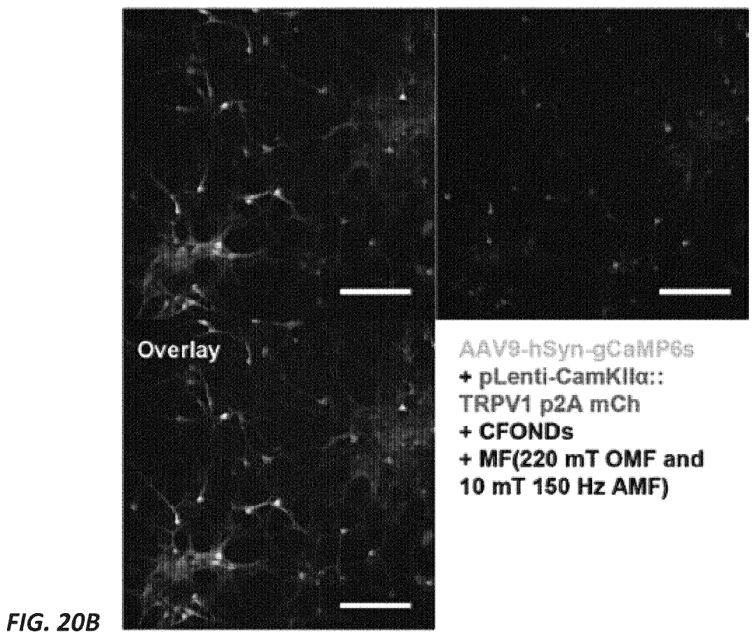

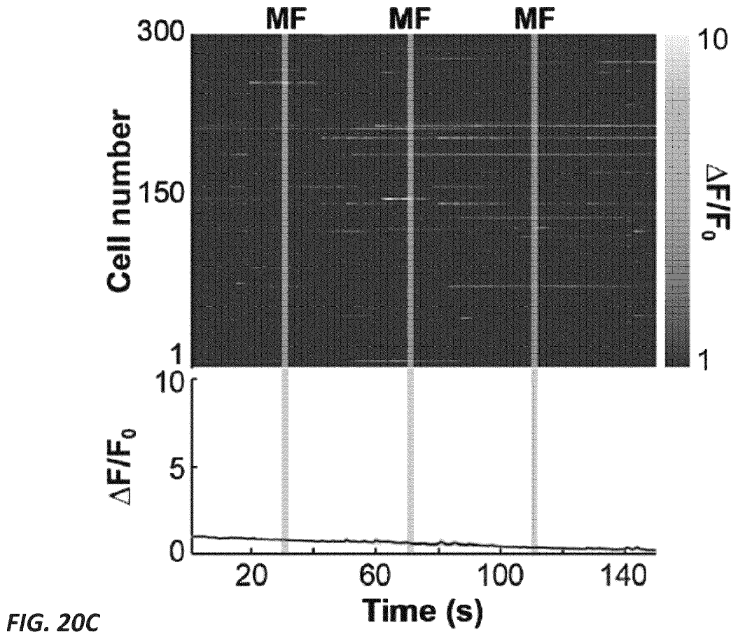

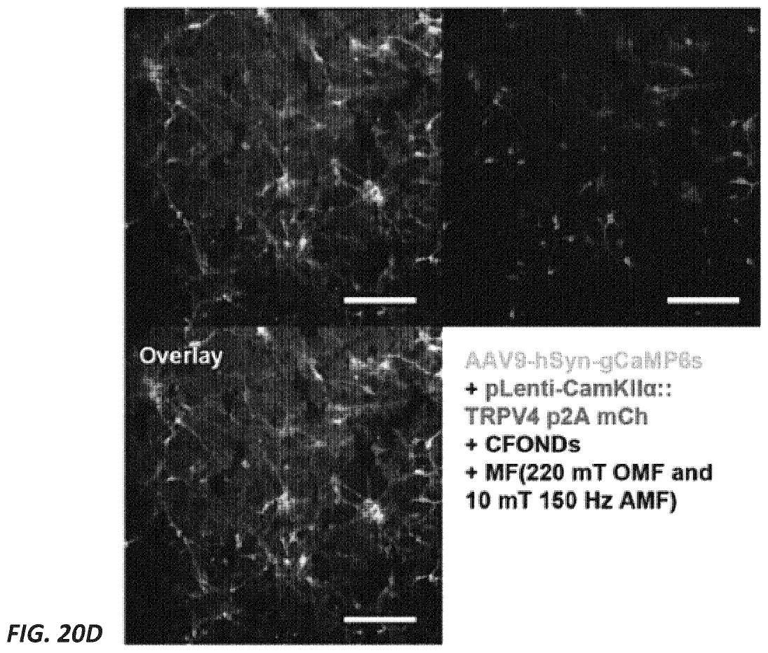

Combined MF conditions employed for ME neuromodulation (e.g., 220 mT OMF; 10 mT, 150 Hz AMF) may not evoke mechanical or thermal effects on neurons due to the inability of the MENDs 100 to oscillate in the presence of a near-saturation OMF and/or dissipate observable heat at AMF frequencies below the kilohertz range. Additionally, the combined MF conditions did not evoke excitation in neurons decorated with CFONDs 150 (that constitute magnetic core 110 and inner shell 120 of MENDs 100) even in the presence of virally delivered thermosensitive ion channels (e.g., transient receptor potential vanilloid receptor 1, TRPV1) as shown in FIGS. 20A and 20B or mechanosensitive ion channels (e.g., TRPV4,) as shown in FIGS. 20C and 20D.

### Mechanistic Investigation of MEND-Mediated Neuromodulation

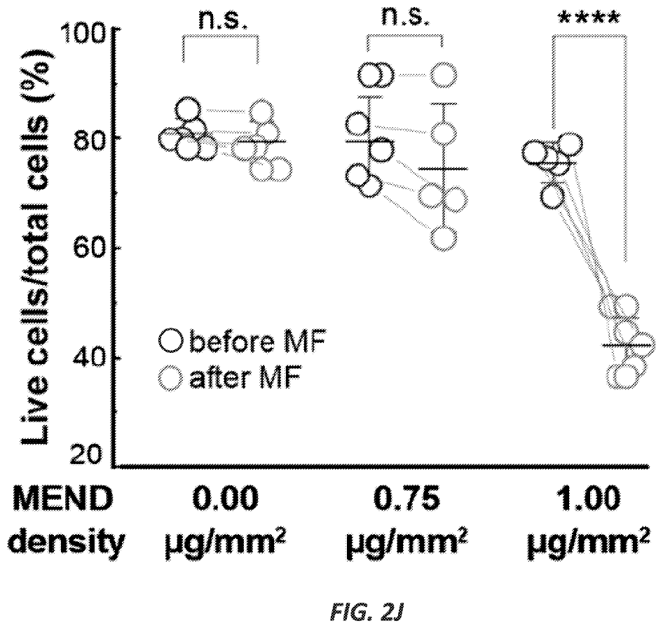

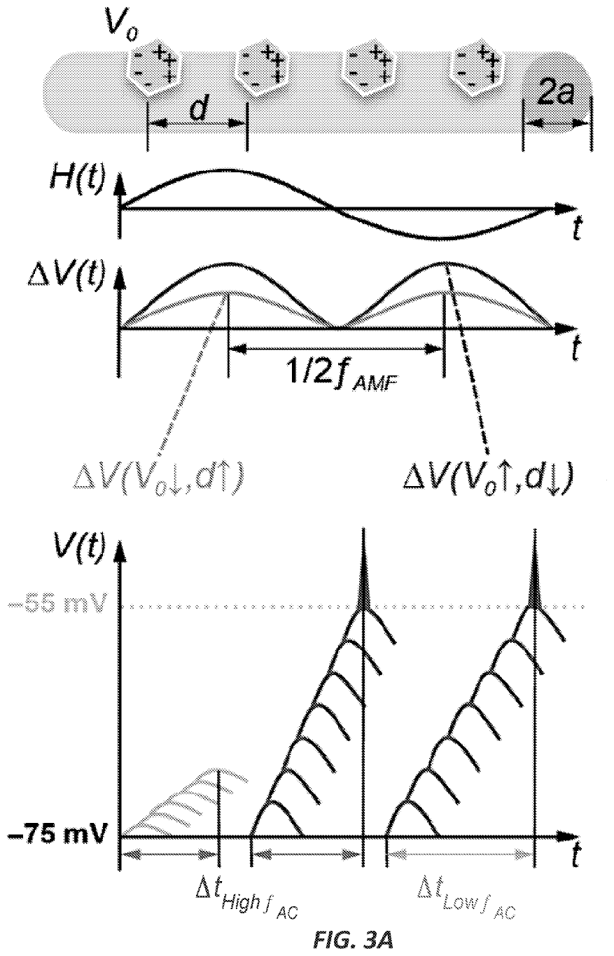

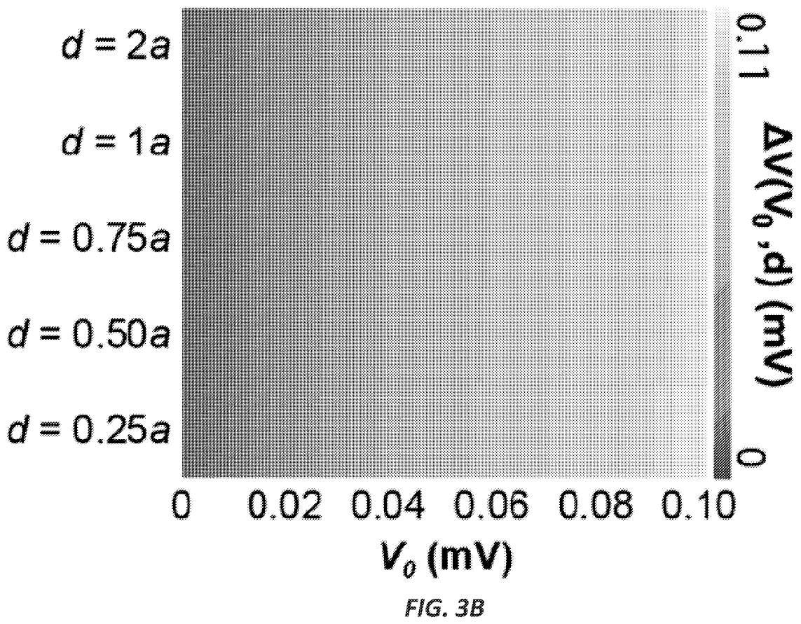

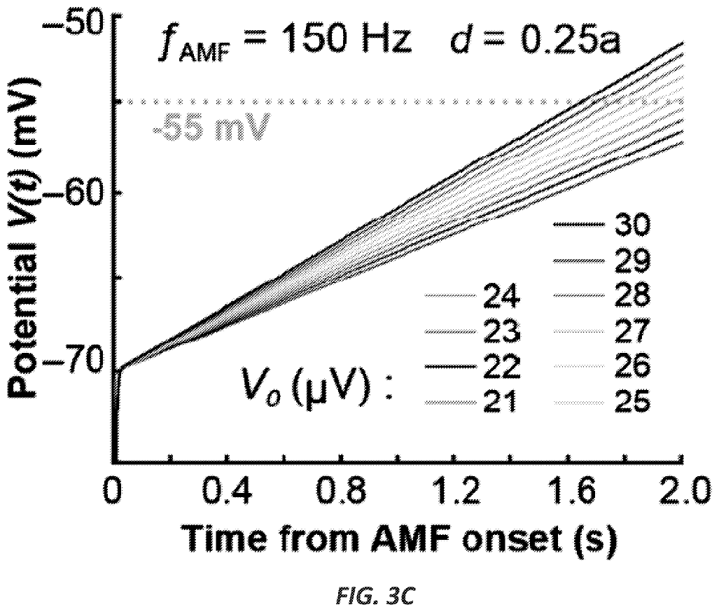

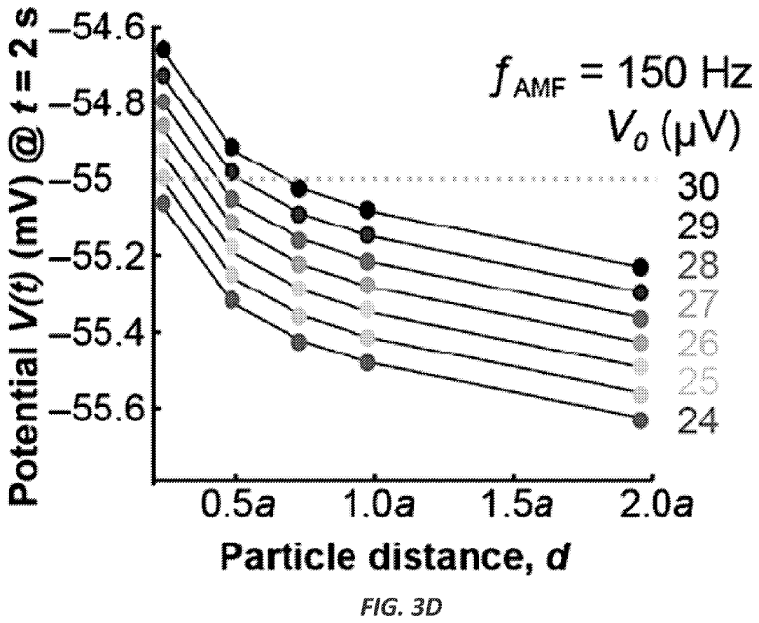

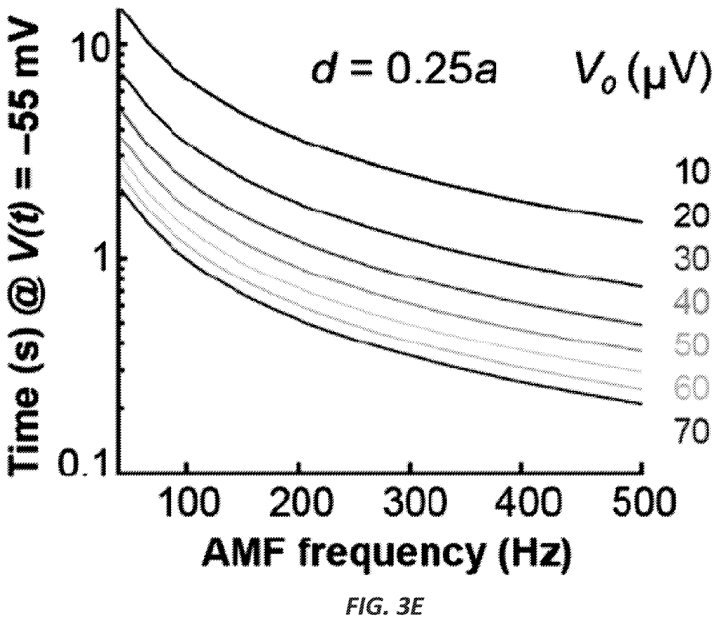

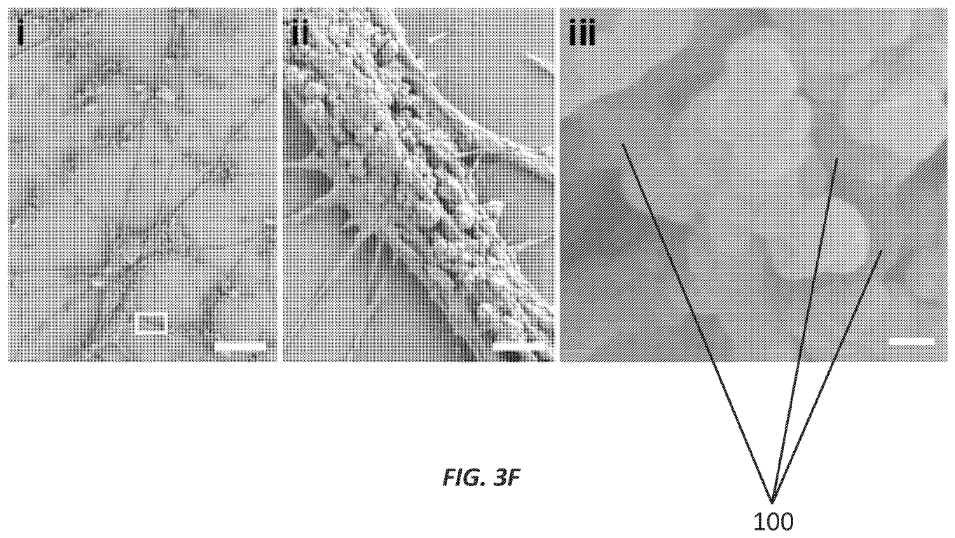

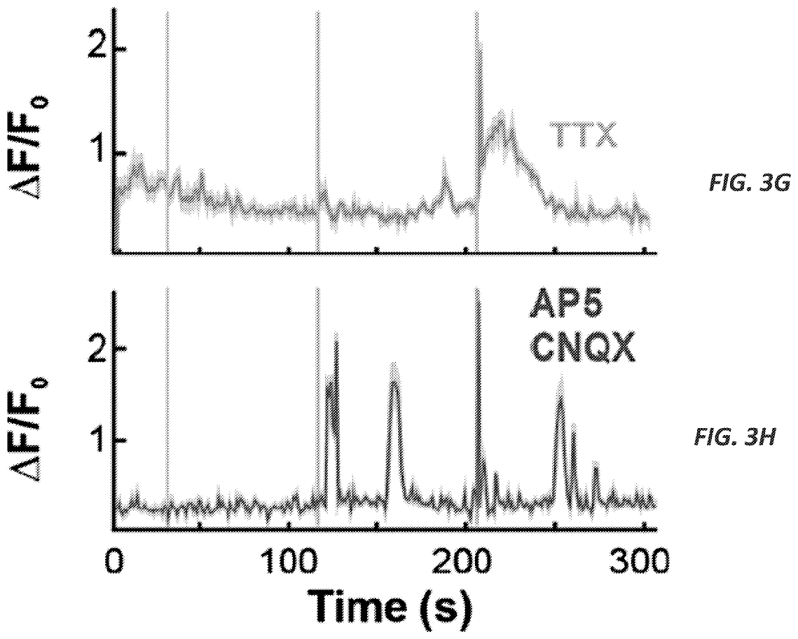

The potential generated by individual MENDs 100 estimated from α_{ME} measurements at the field conditions employed in the in vitro studies was approximately 35 μV, which is below the neuronal activation threshold of approximately 15-30 mV. Consequently, the mechanisms underlying the observed neuronal responses were investigated. MENDs 100 may exert repetitive subthreshold stimulation on neuronal membranes, which is then integrated across multiple periods of the AMF resulting in neuronal depolarization facilitation as illustrated in FIG. 3A and described in the section below entitled Magnetoelectric Coefficient).

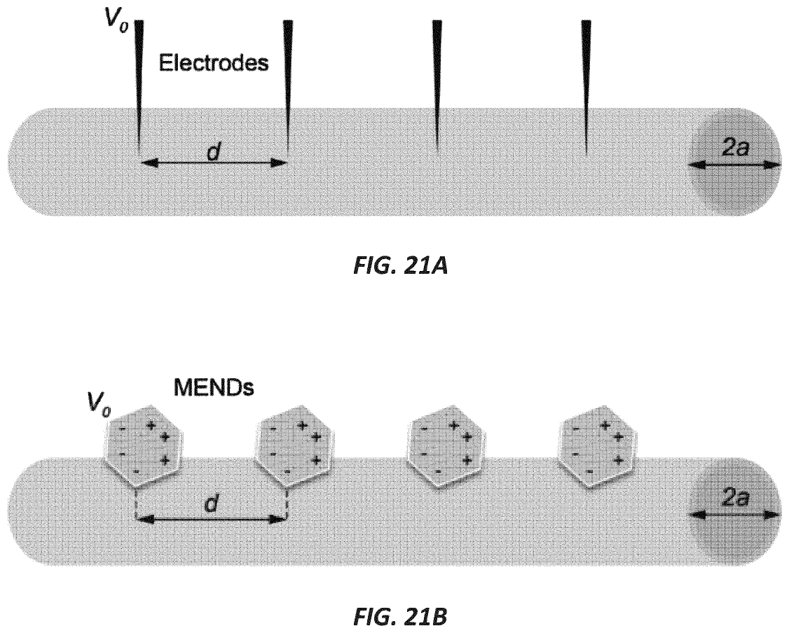

To simulate neuronal facilitation during ME stimulation mediated by MENDs 100, a model of repetitive neuronal excitation was combined with the steady-state solution for the three- dimensional (3D) cable theory (Magnetoelectric Coefficient section below, FIGS. 21A and 21). For a single period of AMF, the integrated change in membrane voltage ΔV was calculated using cable theory as a function of a single MEND 100 potential V₀ and the average intra-particle distance, d (see FIG. 3B). ΔV may depend more strongly on V₀ than on the inter-particle distance. An approximately 10% increase in ΔV was achieved by an 8-times decrease in the particle spacing. The change in membrane potential was then superimposed onto the prior state of the membrane potential, which depended on the stimulation history, stochastic fluctuations, and the duration of the refractory period and hyperpolarization delay for a given neuron (see section entitled Magnetoelectric Coefficient).

FIG. 3C illustrates the change in membrane potential over time produced by the AMF oscillating at 150 Hz for MENDs 100 with d=0.25a (a is axonal radius) and various values of Vo. These findings indicate that neuronal potential (initially set to −70 mV) may reach threshold values of −55 mV in 2 seconds if the MENDs 100 are spaced by 0.25a and generate V₀>24 μV on the membrane, which is consistent with the observed results shown in FIGS. 2D-2F.

The model disclosed herein employs the steady-state solution for the cable equation and temporal summation of the membrane potential fluctuations. This approximation was motivated by the same order of magnitude of the duration of AMF pulses (e.g., about 6.67 milliseconds at 150 Hz) and the neuronal time constant (e.g., about 1-20 milliseconds). At lower frequencies (e.g., <100 Hz), AMF pulse length may preclude the superposition of generated potentials ΔV, which may further explain lower rates of neuronal responses at these frequencies (see FIGS. 16A-16C). As the model does not recapitulate the neuronal conduction block, the latency to reach the threshold potential monotonically may decrease with increasing V₀ (see FIG. 3D) and AMF frequency (see FIG. 3E). This decrease may contrast with the experimental observations at AMFs with frequencies >150 Hz.

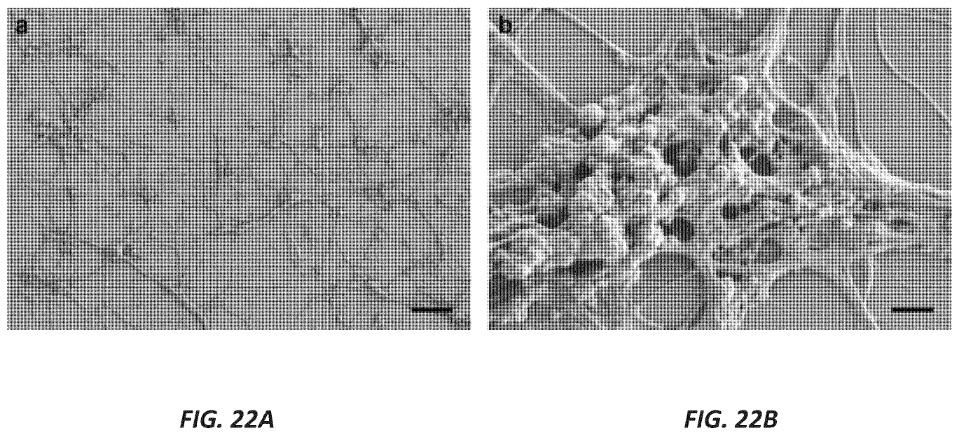

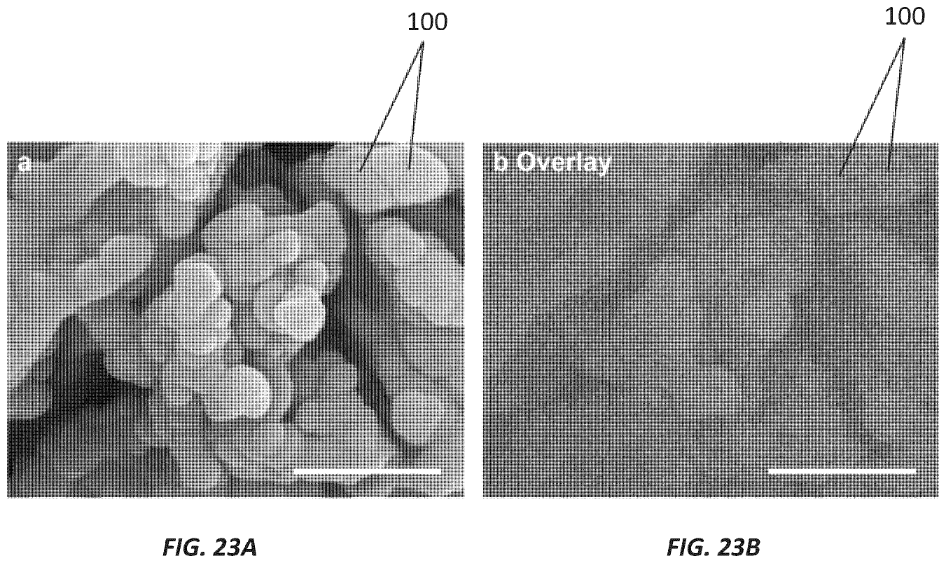

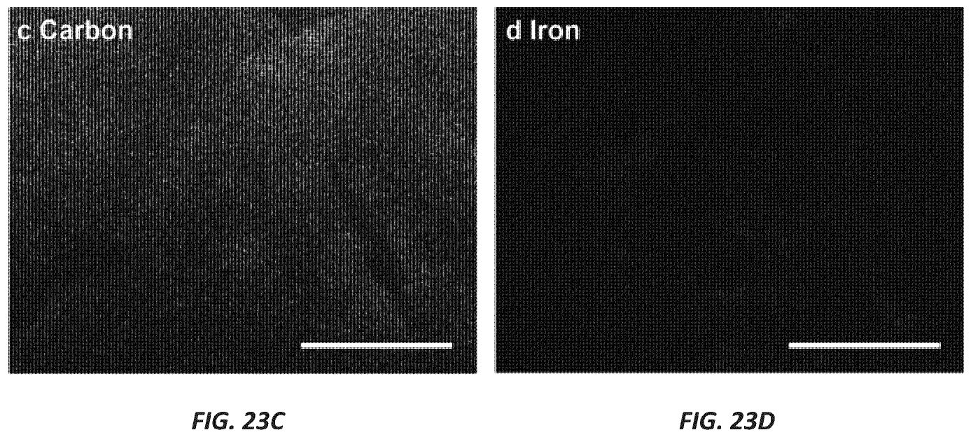

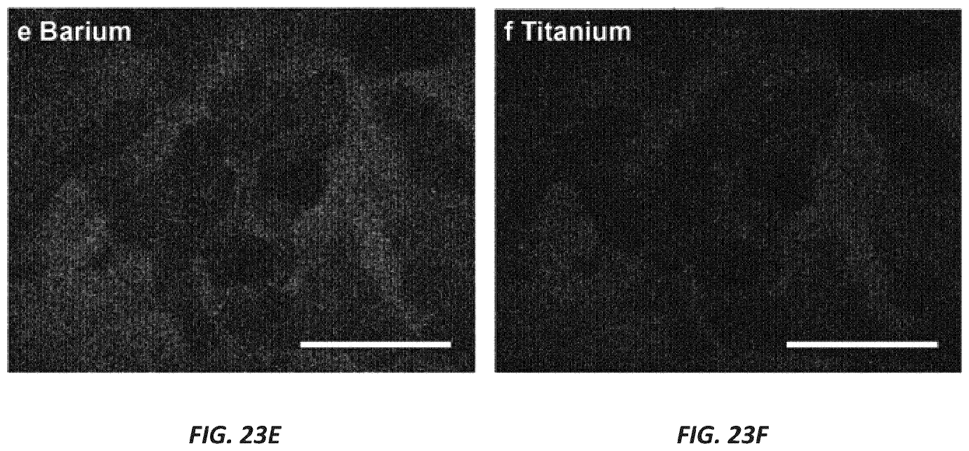

Another assumption of the model was the uniform distribution of MENDs 100 on neuronal membranes (e.g., membrane 106). To test the validity of this assumption, scanning electron microscopy (SEM) was applied to the MEND-decorated neurons and is illustrated in FIG. 3F. Since synaptic boutons in intact neurons (shown in FIGS. 22A and 22B) exhibit dimensions comparable to MENDs 100, energy- dispersive X-ray spectroscopy mapping was employed to distinguish the particles from neuronal structures by their element content and is shown in FIGS. 23A-23F, revealing the presence of individual MENDs 100 as well as few-particle aggregates on the cell membranes.

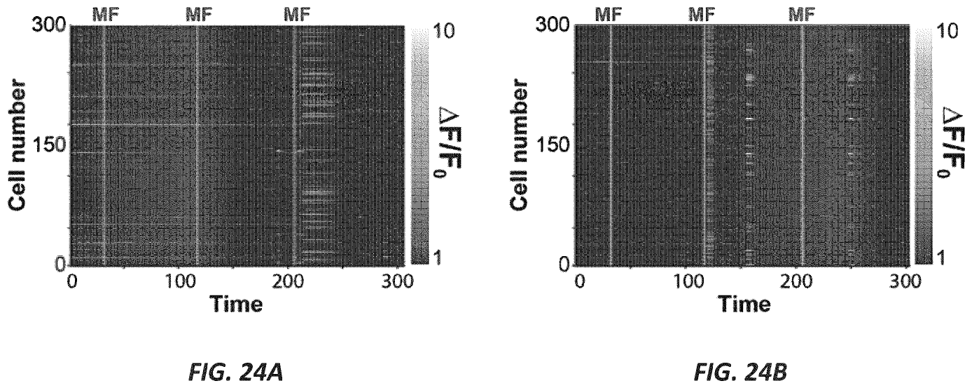

Consistent with the model disclosed herein, neuronal depolarization, may be observed even in the presence of the voltage-gated sodium channel blocker tetrodotoxin (e.g., TTX at 1 μM), as the threshold potential could be reached regardless of the state of the sodium channels (see FIGS. 3G and 24A) at the density of approximately 0.75 μg/mm² MENDs 100. Moreover, MEND-mediated excitation may not rely on synaptic transmission within the neuronal network, as GCaMP6s transients may be observed in response to a MF (e.g., 220 mT OMF; 10 mT, 150 Hz AMF) in the presence of a cocktail of the α-amino-3-hydroxy-5-methyl-4-isoxazolepropionic acid (AMPA) receptor antagonist ((2R)-amino-5-phosphonovaleric acid, AP5, 100 μM) and the non-N-methyl- D-aspartate (non-NMDA) receptor antagonist (6-cyano-7-nitroquinoxaline-2,3-dione, CNQX, 20 μM) as shown in FIGS. 3H and 24B.

Without being bound by any particular theory or hypothesis, the model offers qualitative description of possible mechanisms underlying MEND-mediated neural excitation and provides a framework for MEND-mediated modulation in vivo.

### MEND-Mediated Neuromodulation In Vivo

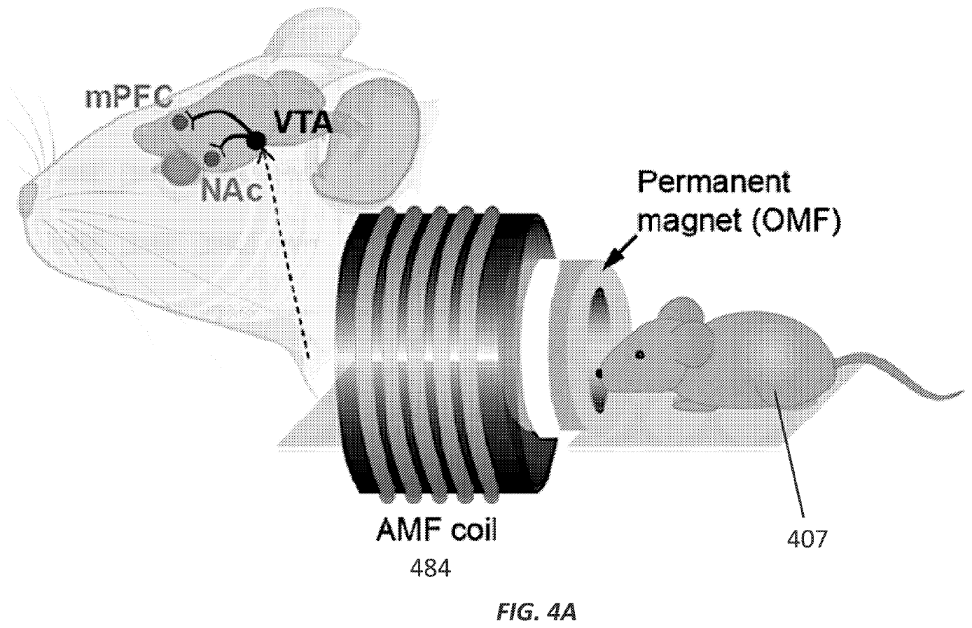

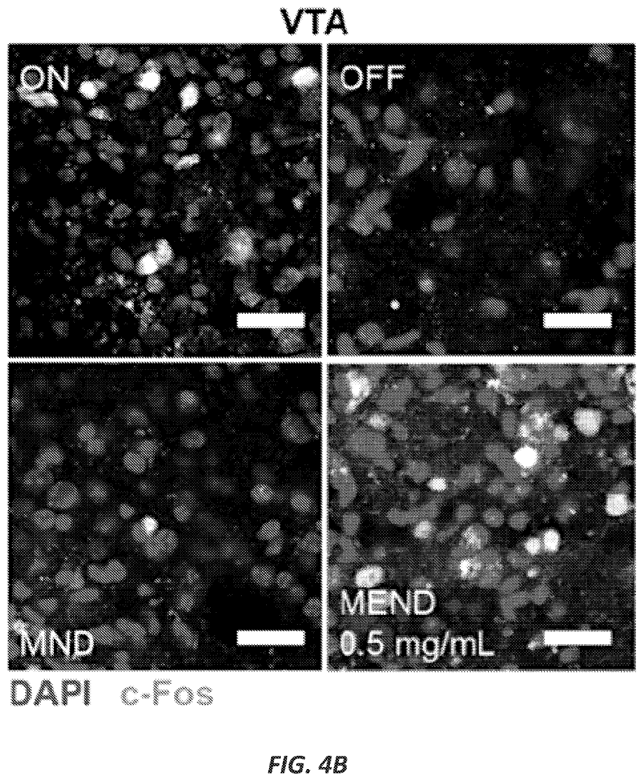

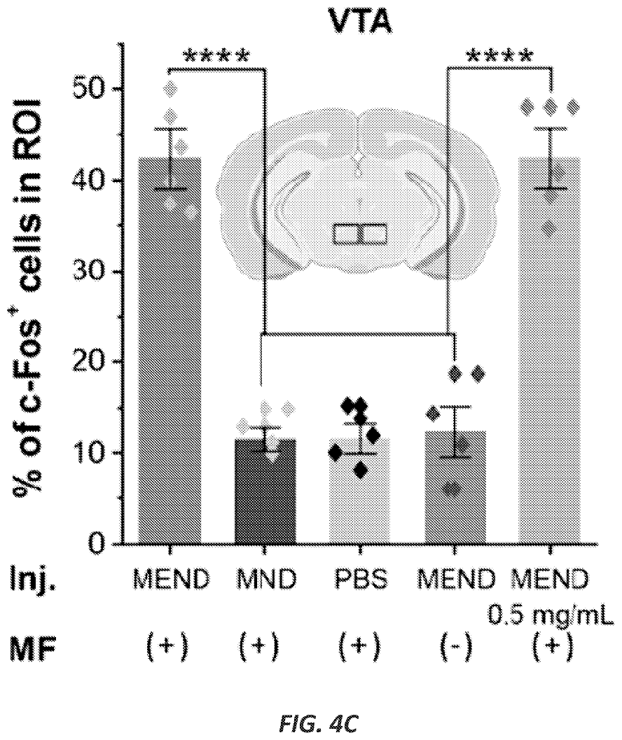

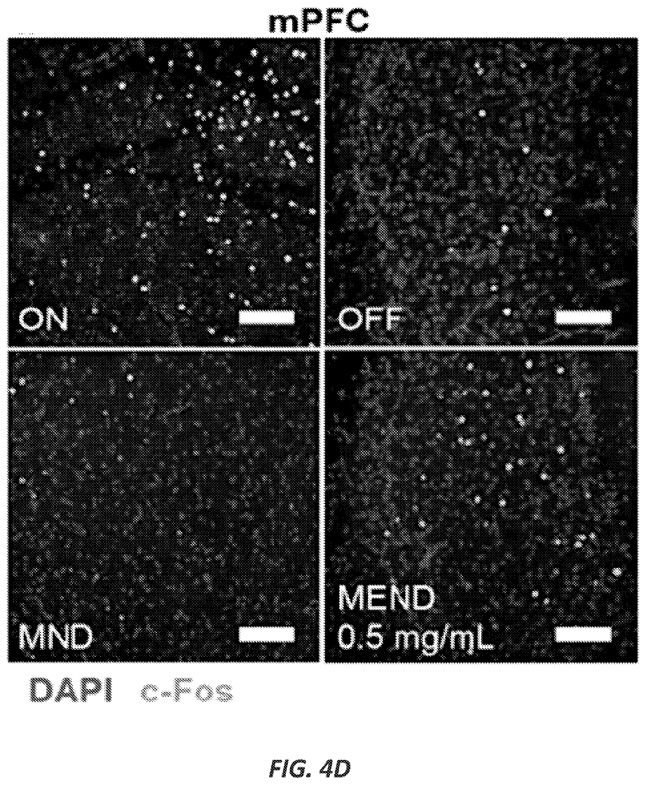

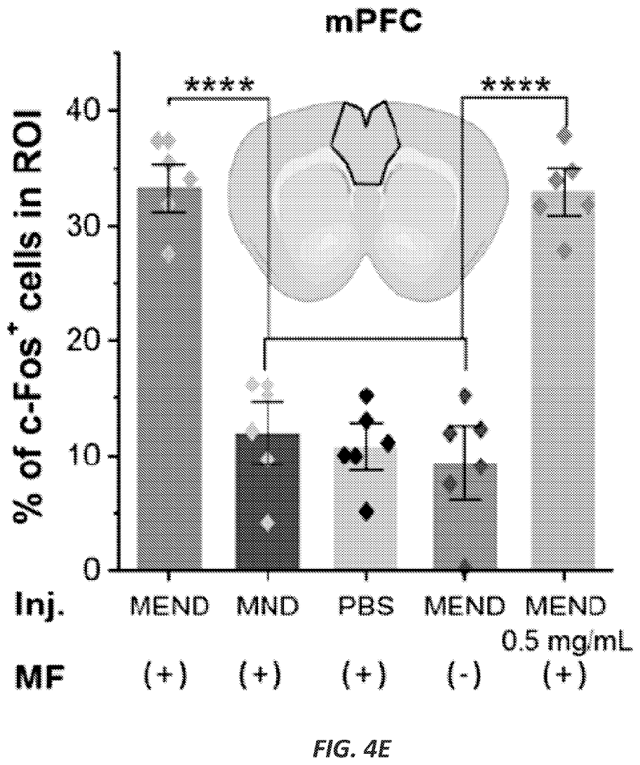

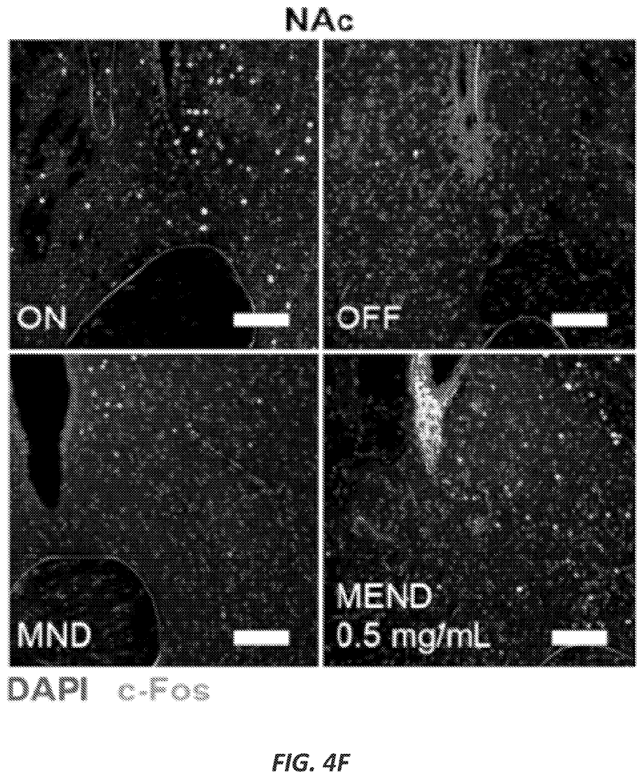

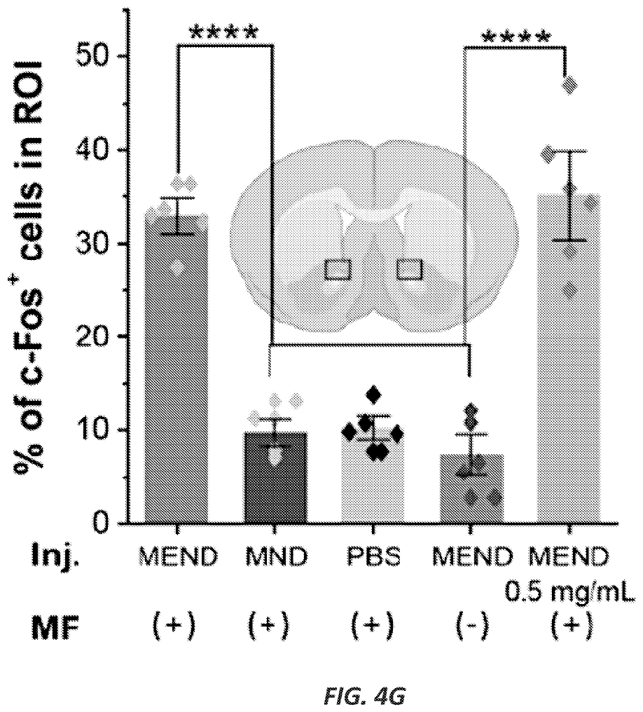

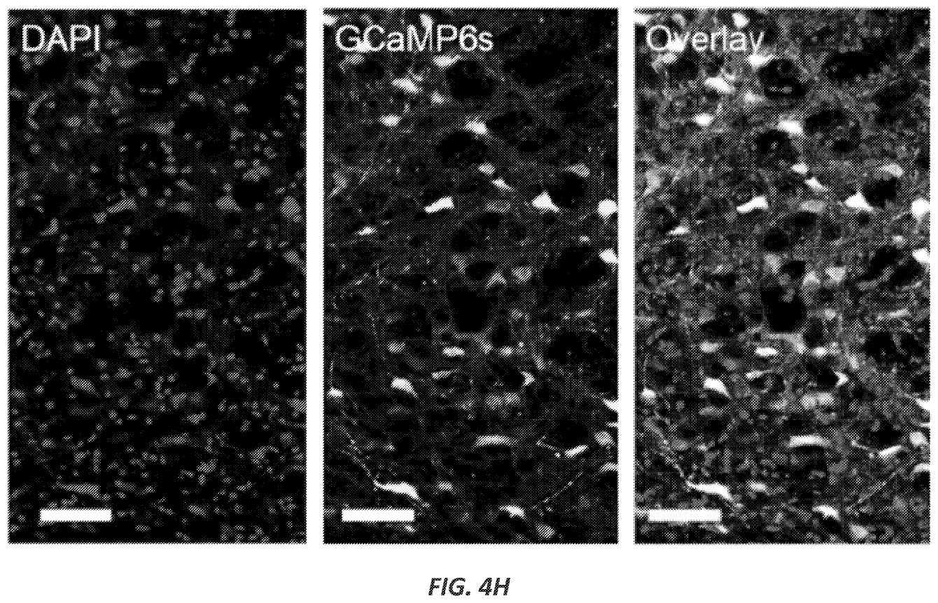

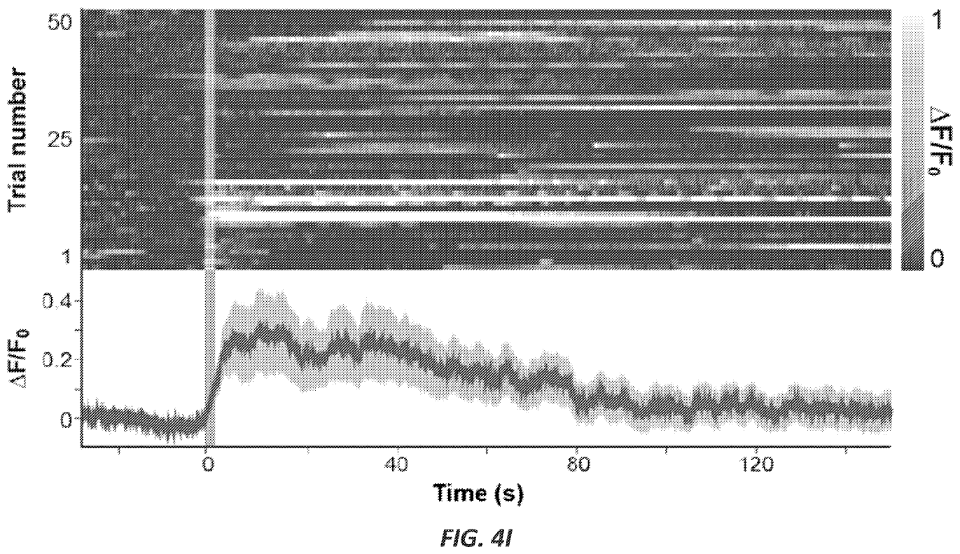

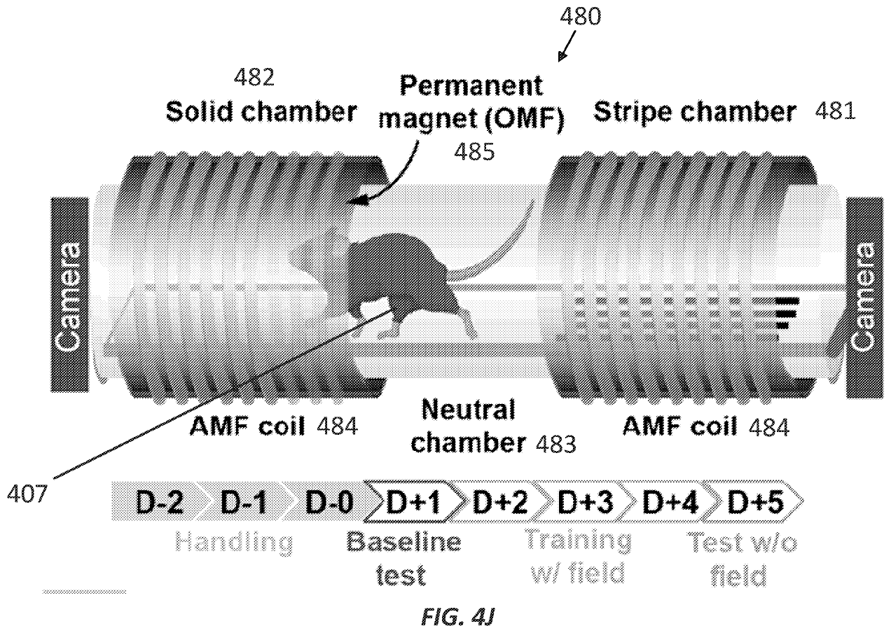

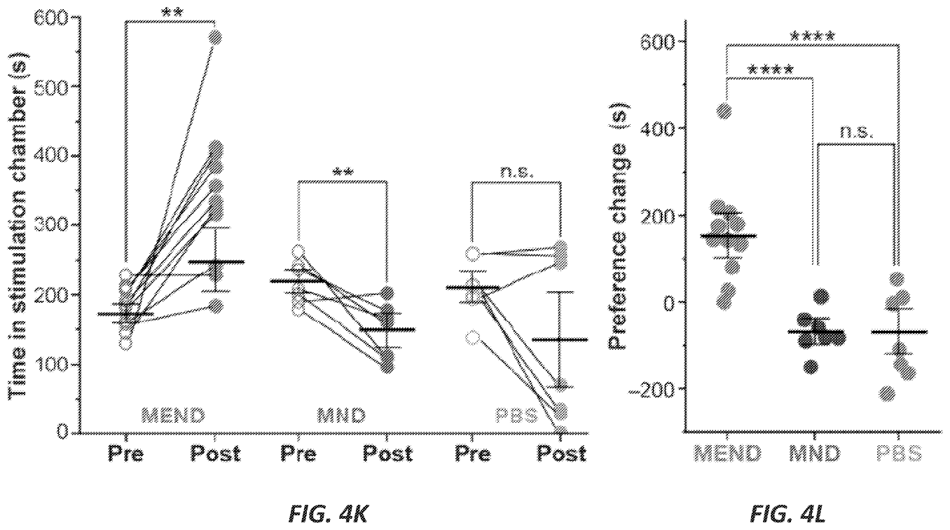

FIG. 4A illustrates a schematic of MEND-mediated neuromodulation in deep-brain structures in mice 407. MENDs 100 (1.5 μL volume, 1.5 mg/mL or a control solution) were injected into the left ventral tegmental area (VTA), a predominantly dopaminergic structure critical to reward processing, in wild-type BL6/57 mice 407 (n=6 for every sample group). Following a 1-week recovery period, the mice were subjected to 3 cycles of MF stimulation (220 mT OMF, 10 mT, 150 Hz AMF; 2-second epochs separated by 90-second rest periods) via an AMF coil 484, and their brain tissue was analyzed with respect to expression of an immediate early gene c-Fos, a marker of neuronal activity (described further below).

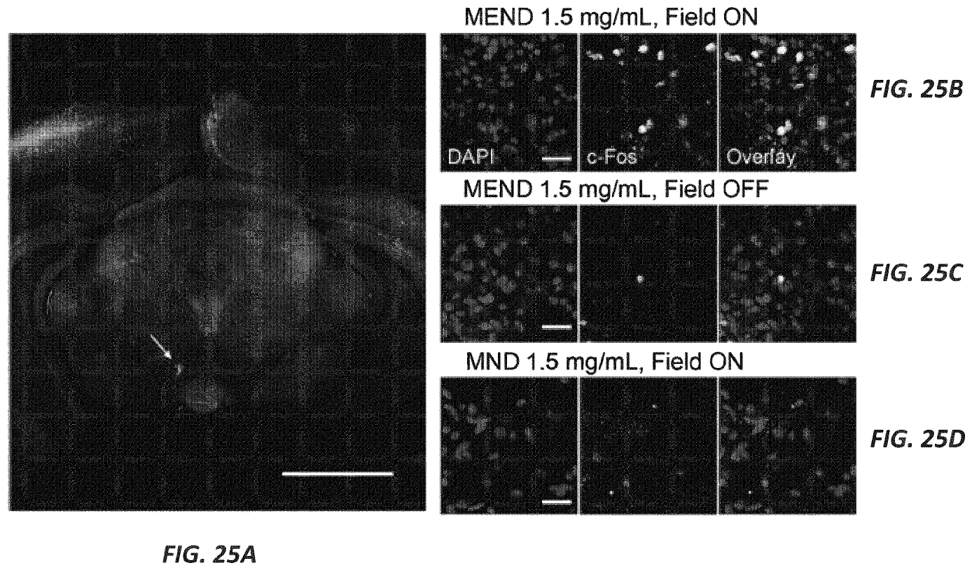

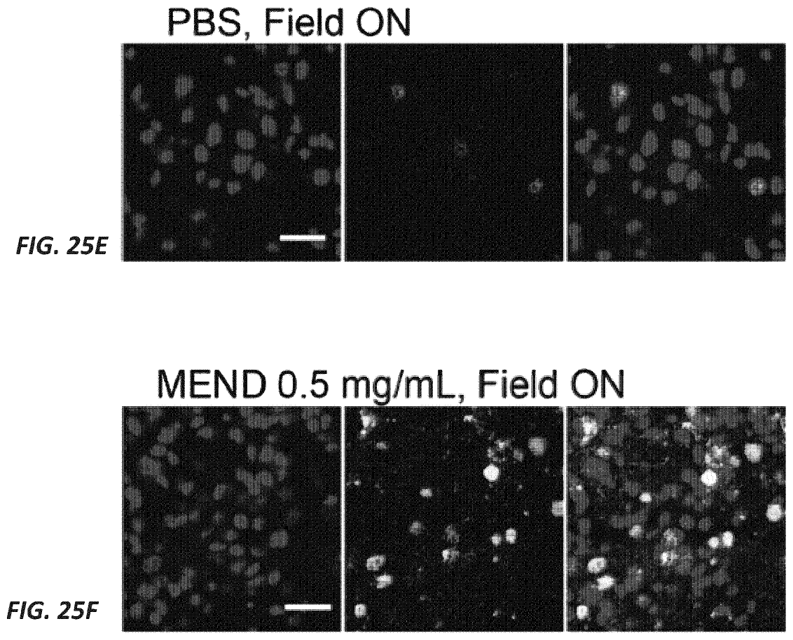

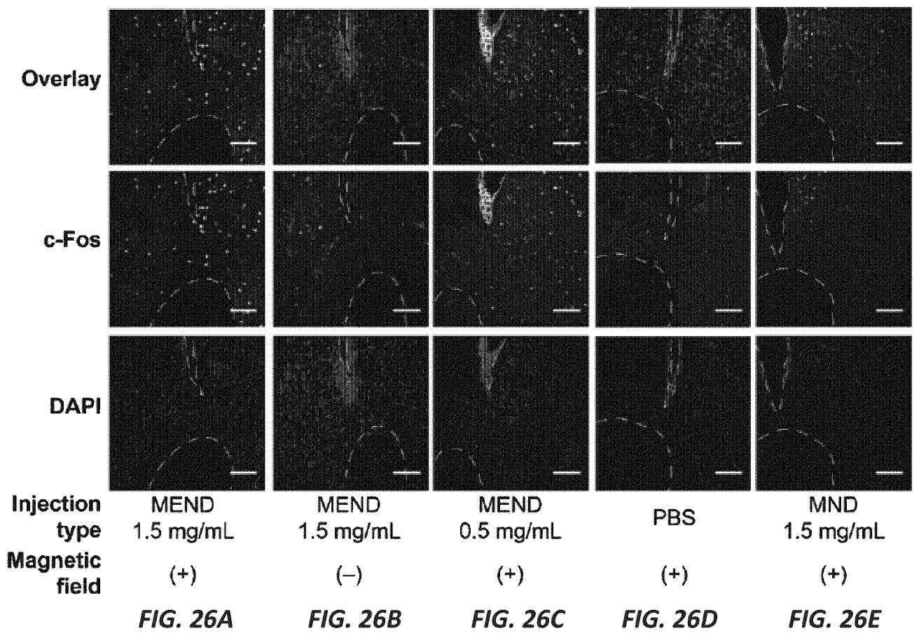

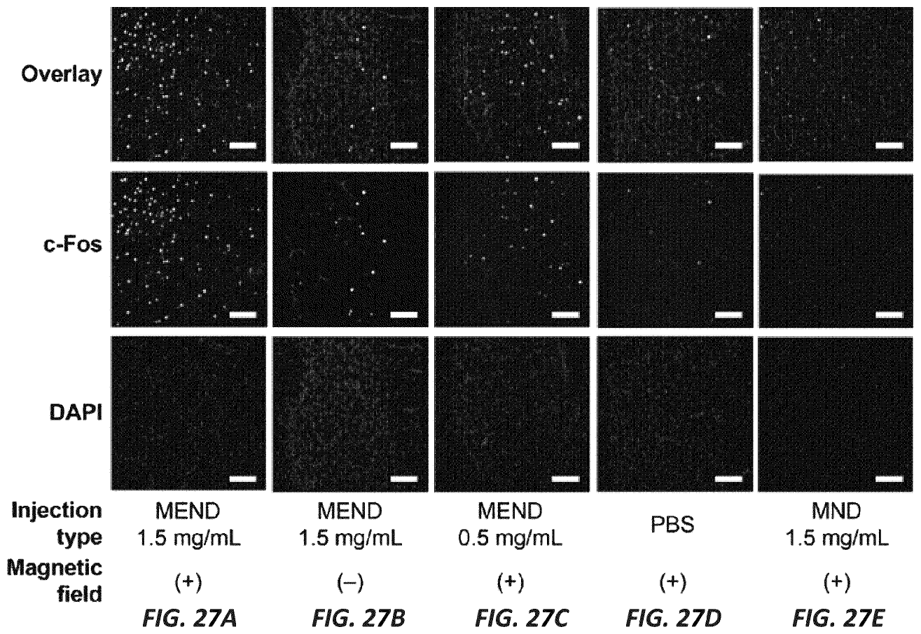

Consistent with the analyses in vitro disclosed above, the percentage of c-Fos expressing cells relative to all cells in a field of view marked by nuclear stain 4′,6-diamidino-2-phenylindole (DAPI) was significantly higher in the VTA of mice 407 injected with MENDs 100 and exposed to the MF in comparison to controls injected with phosphate buffered saline (PBS), MND cores 110 without the piezoelectric BaTiO₃ shell (1.5 μL at 1.5 mg/mL), or mice 407 injected with MENDs 100 and not exposed to the MF as shown in FIGS. 4B, 4C, 25A-25F. The expression of c-Fos in response to MF stimulation was also significantly higher in the nucleus accumbens (NAc) and the medial prefrontal cortex (mPFC), the excitatory targets of the VTA, in mice 407 injected with MENDs 100 compared to the controls as shown in FIGS. 4D-4G, 26A-26E, and 27A-27E. Reducing the MEND 100 concentration to values comparable to the experiments in vitro (e.g., 1.5 μL at 0.5 mg/mL) resulted in c-Fos expression levels in the VTA, NAc, and mPFC similar to those found with higher particle concentrations as shown in FIGS. 4B-4G, 25A-25F, 26A-26E, and 27A-27E.

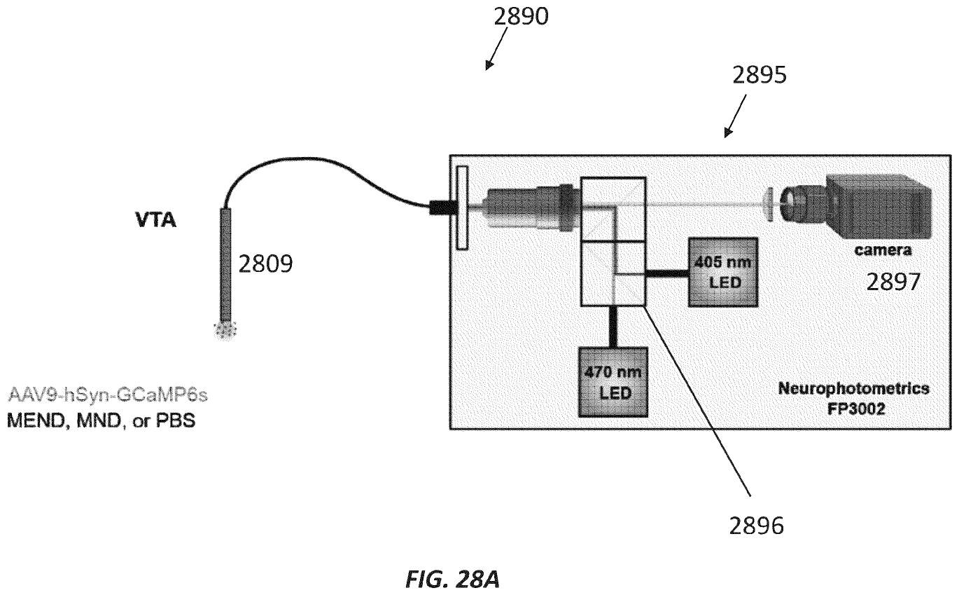

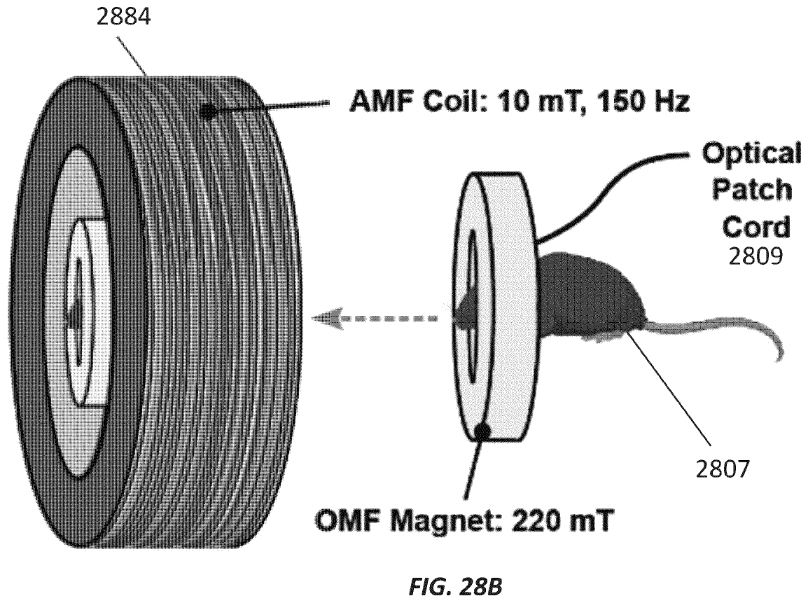

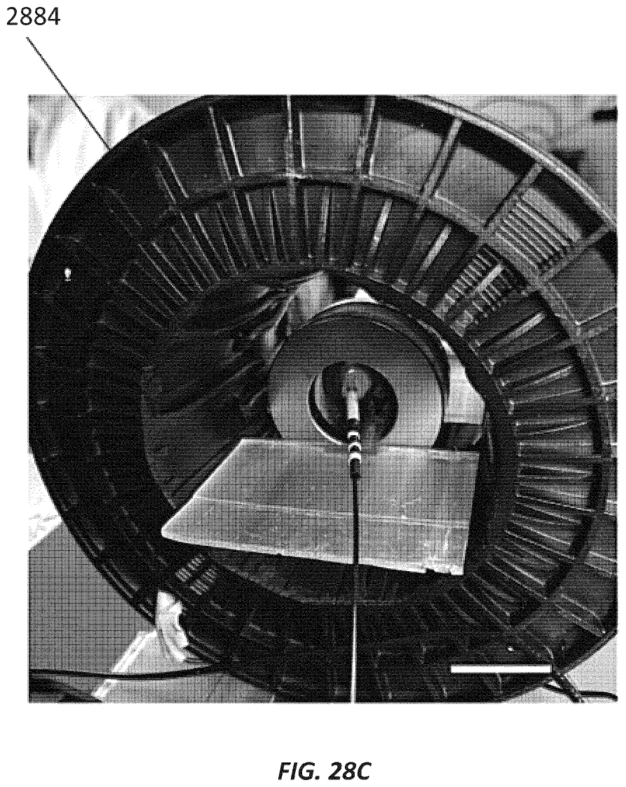

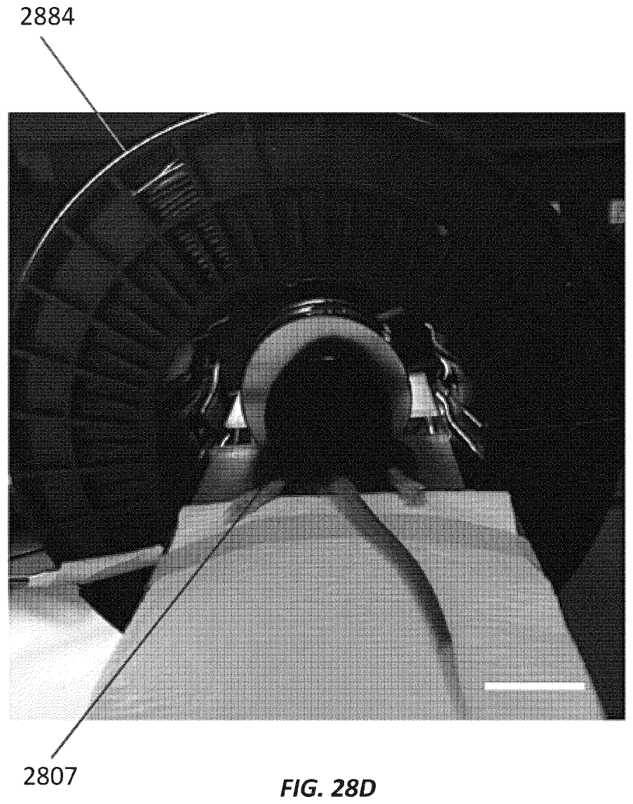

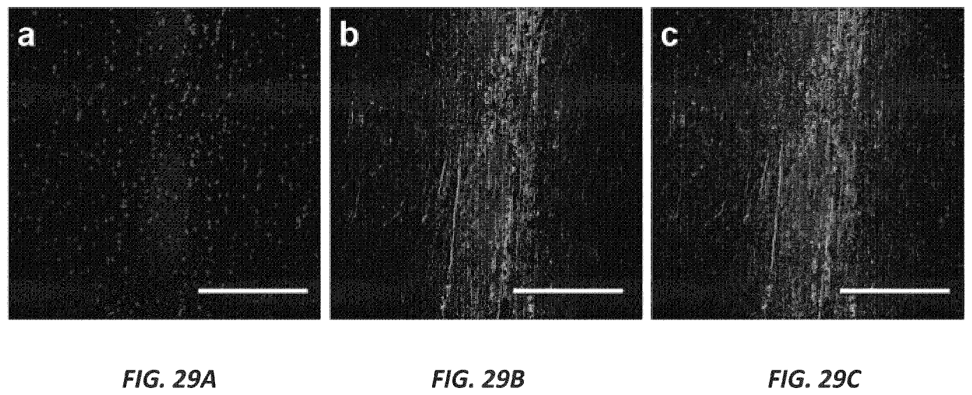

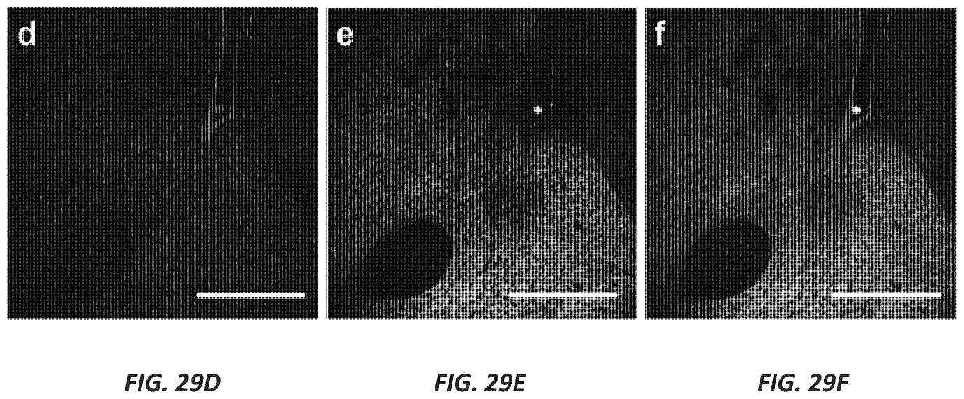

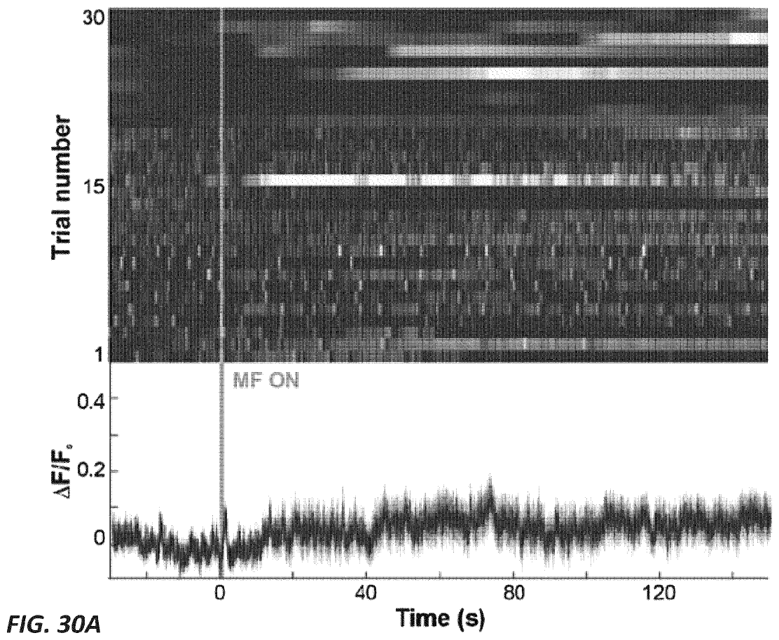

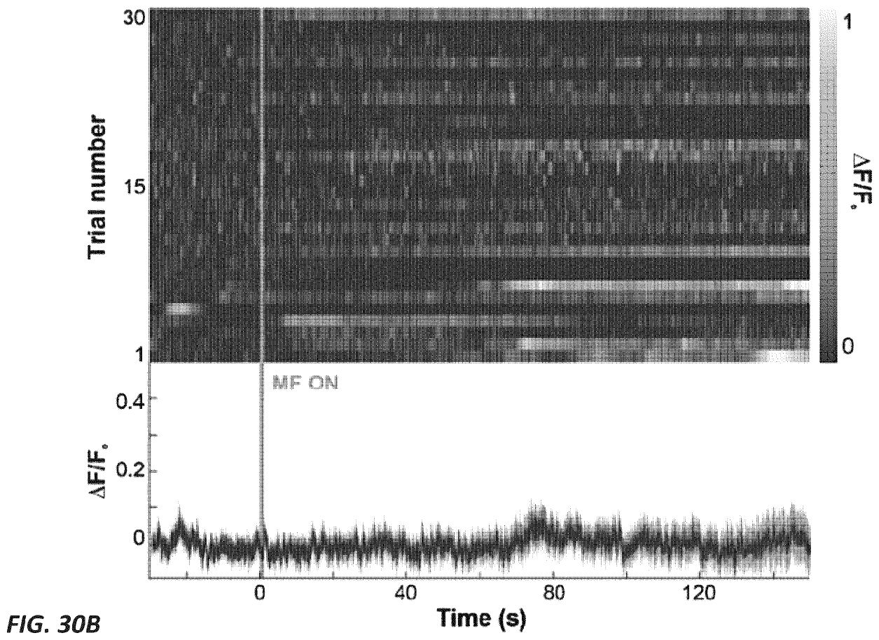

FIGS. 28A-28D illustrate the fiber photometry system 2890. The fiber photometry system 2890 may include a light-emitting device (LED) 2895 and a silica optical fiber 2809. fiber photometry was employed in the VTA to record neural activity during ME modulation in mice. A MEND 100 (e.g., 1.3 μL, 1.5 mg/mL) solution (or control MNDs cores 110 or PBS) were injected into the VTA of wild type mice 2807, along with the adeno-associated virus carrying GCaMP6s under a pan-neuronal human synapsin promoter (AAV9-hSyn::GCaMP6s, 0.3 μL), which was followed by an implantation of a 200 μm silica optical fiber 2809 into the same location. Fluorescent imaging confirmed GCaMP6s expression in cell bodies in the VTA, and in axons projecting to NAc and mPFC as shown in FIGS. 4H and 29A-29F. Following a 2-week incubation period, GCaMP6s fluorescence increase was observed in response to 2-second MF stimulation (e.g., 220 mT OMF, 10 mT, 150 Hz AMF) via a AMF coil 2884 in mice 2807 injected with MENDs 100 but not in those injected with control particles or PBS as shown in FIG. 4I, 30A, and 30B.

Based on dopamine-dependent reward circuits, it was tested whether ME stimulation in the VTA could induce place preference in a three-chamber assay in mice. FIG. 4J illustrates a schematic of the place preference behavioral apparatus 480 (top) and experimental timeline (bottom). To enable neural excitation mediated by MENDs 100, a custom place preference arena comprised of two stimulation chambers with different floor patterns (stripes 481 vs. solid 482) equipped with identical electromagnets was designed to supply the AMF (e.g., via a AMF coil 484) and permanent magnets 485 supplying the OMF separated by a neutral chamber 483. A mouse 407 may be placed in the behavior apparatus 480.

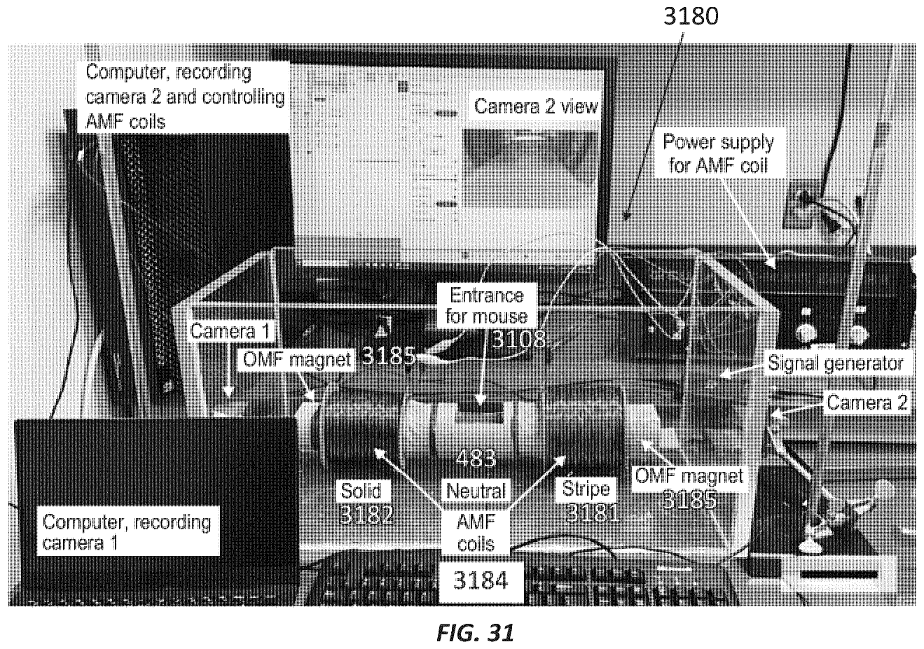

FIG. 31 is a photograph of the behavioral apparatus or arena 3180 employed for place-preference assays enabling application of magnetic fields necessary for magnetoelectric neuromodulation. The arena 3180 may include a stripe stimulation chamber 3181, a solid stimulation chamber 3182, and a neutral chamber 3183 separating the stripe stimulation chamber 3181 and the solid stimulation chamber 3182. The arena 3180 may also include an AMF coil 3184 and an OMF magnet 3185. The area 3180 may also include an entrance 3108 for a mouse.

Following a 1-week recovery period, mice injected with MENDs 100 (e.g., 1.5 μL, 1.5 mg/mL), control MNDs 110, or PBS into the VTA were habituated to the arena 3180 for 3 days, and their baseline preference was then tested for 10 minutes on day 1 based on the experimental timeline shown in FIG. 4J bottom panel. During learning days 2-4, the mice were allowed to explore the entire arena 3180 for 10 minutes while an MF (220 mT OMF, 10 mT, 150 Hz AMF, 2-second epochs separated by 90-second rest periods) was applied in their baseline non-preferred chamber.

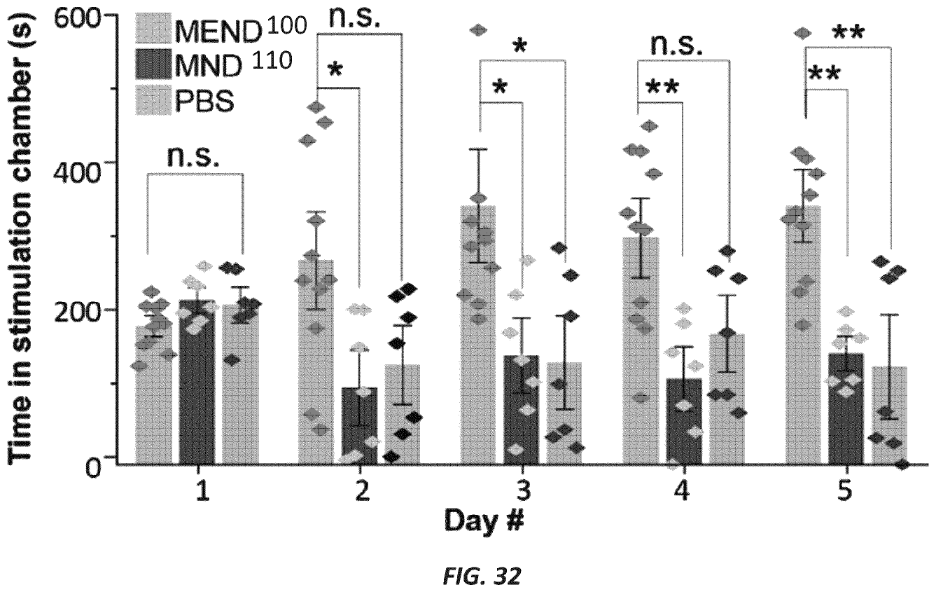

On test day 5, the mice explored the entire arena 3180 in the absence of MF stimuli in either chamber. On days 2-4 learning period, mice injected with MENDs 100 in the VTA exhibited an increased tendency toward the magnetic stimulation chamber 3181 or 3182, as compared to the mice injected with control solutions as shown in FIG. 32. On day 5 (Test), mice injected with MENDs 100 demonstrated a conditioned preference to the stimulation chamber(s) 3181 or 3182 (as compared to Day 1, Baseline), while mice injected with MND 110 and PBS, respectively, exhibited or trended toward aversion for the stimulation chamber(s) 3181 or 3182 as shown in FIG. 4K. The average preference change for the stimulation chamber(s) 3181 or 3182 in MEND-injected mice was significantly greater and positive, while it was slightly negative in mice injected with control solutions as shown in FIG. 4L. This aversion may be attributed to the minor vibrations and noise associated with electromagnetic operating at 150 Hz.

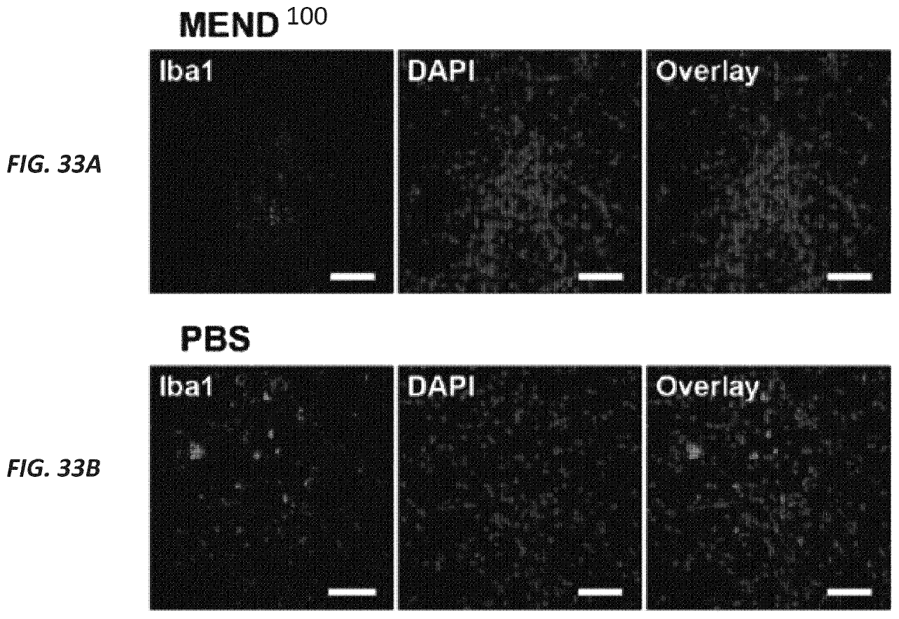

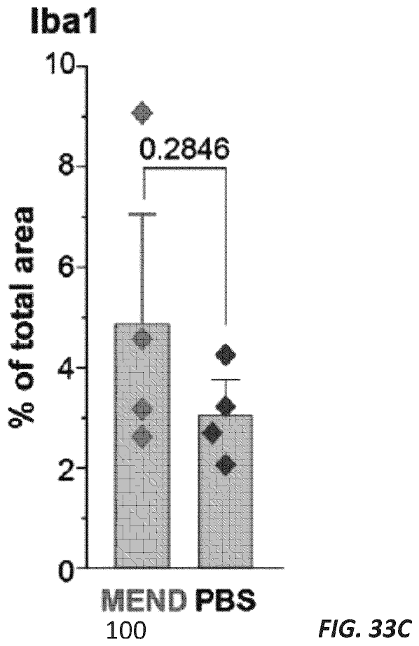

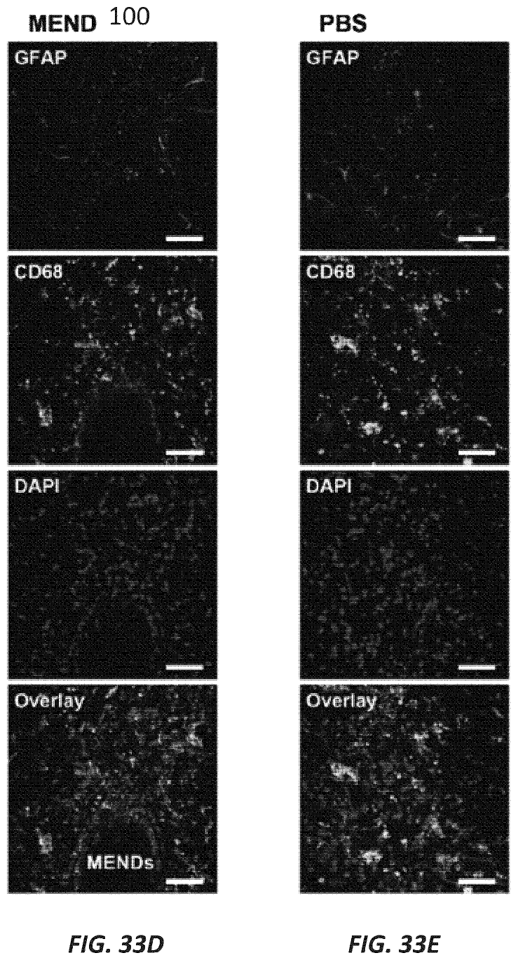

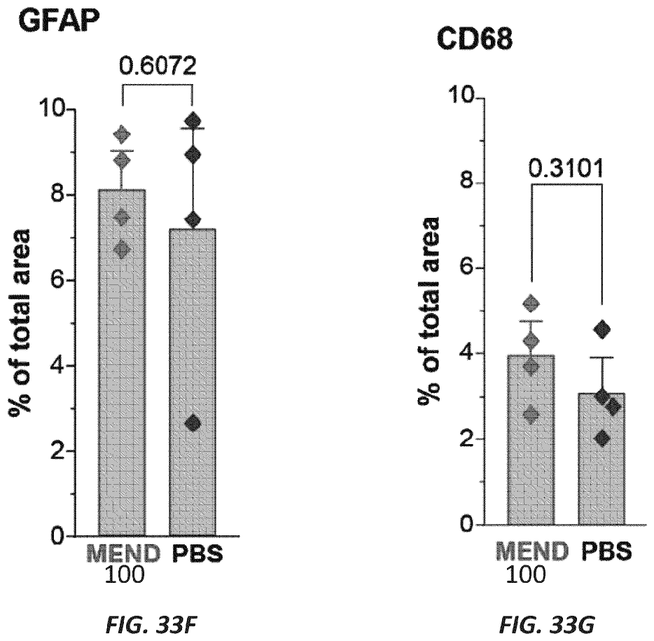

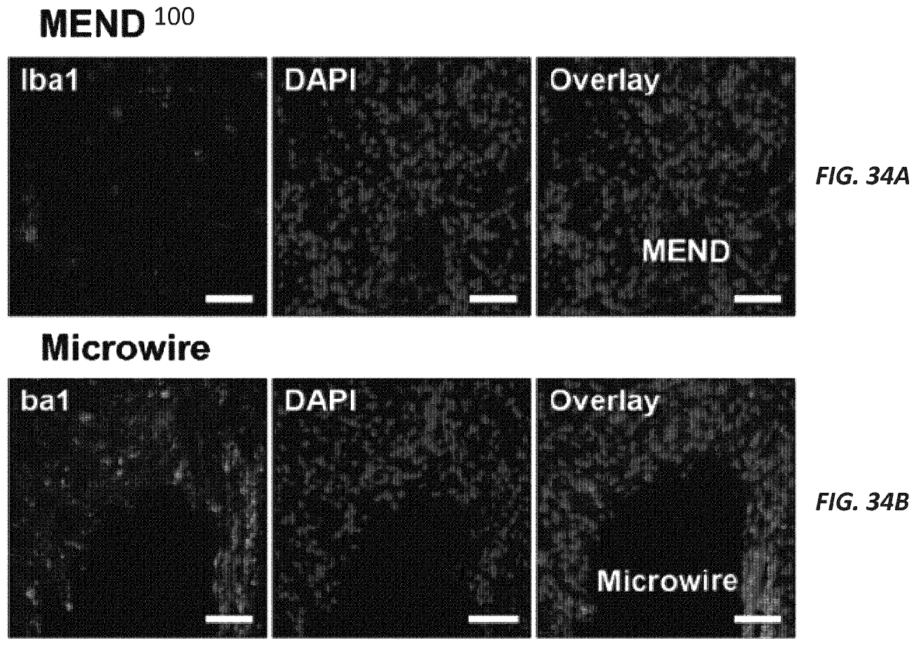

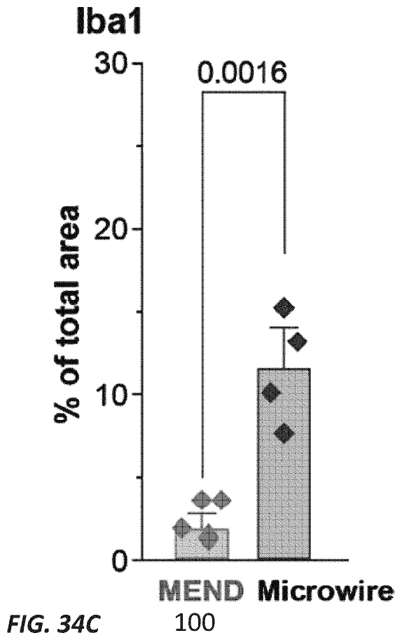

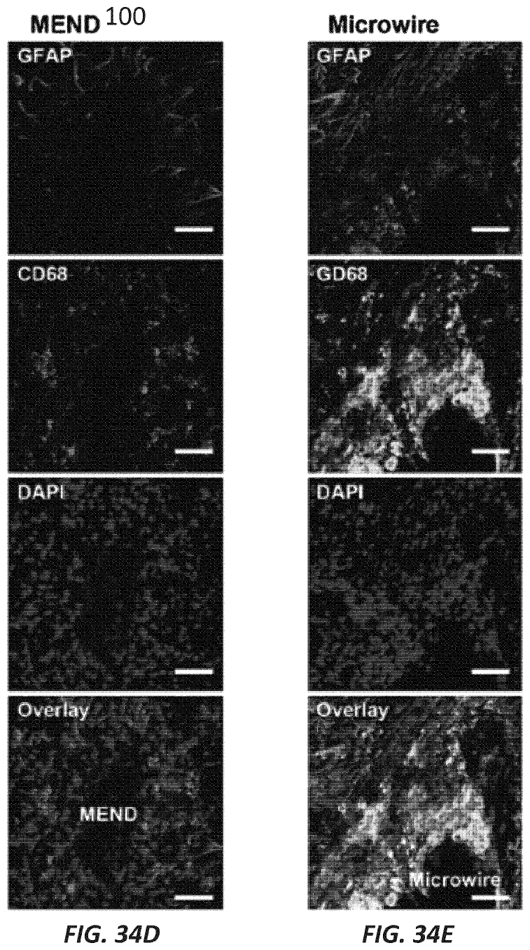

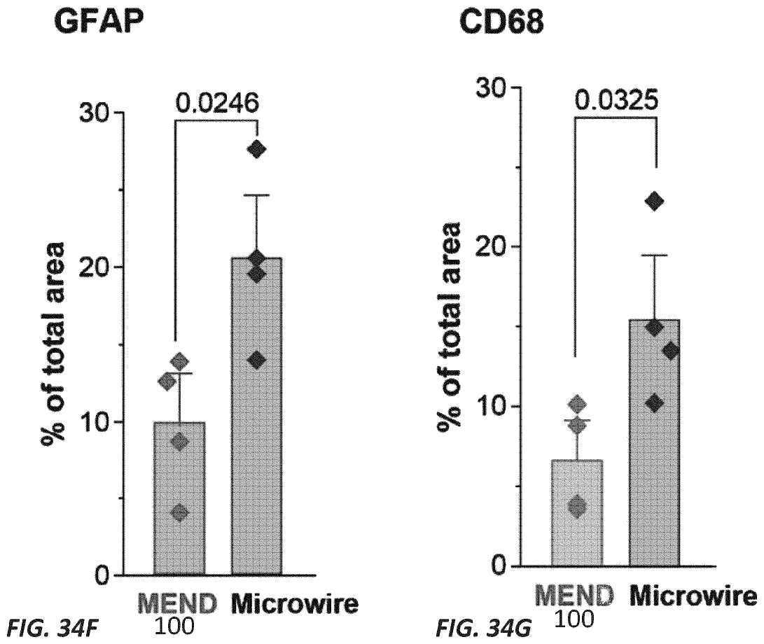

The biocompatibility of MEND-mediated neuromodulation was assessed. It was hypothesized that wireless neuromodulation with MENDs 100 may reduce tissue damage when compared to electrode implantation. A microglial activation marker (e.g., ionized calcium-binding adaptor molecule1, Iba1), astrocytic glial fibrillary acidic protein (GFAP), and macrophage activation marker (e.g., cluster of differentiation 68, CD68) surrounding MENDs 100 relative to PBS injection controls and to 300 μm stainless steel microwire implants commonly used for electrical neuromodulation in mice were quantified. There were negligible increases in Iba1, GFAP, and CD68 expression in mice injected with MENDs 100 compared to those injected with PBS. By contrast, all markers were significantly upregulated near the microwire implants relative to MEND 100 injections as shown in FIGS. 33A-33G and 34A-34G.

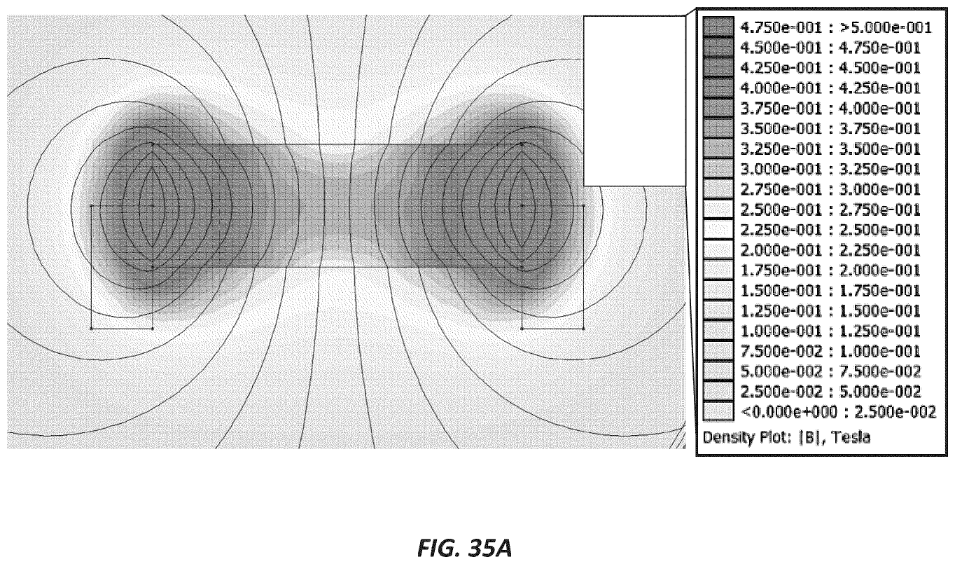

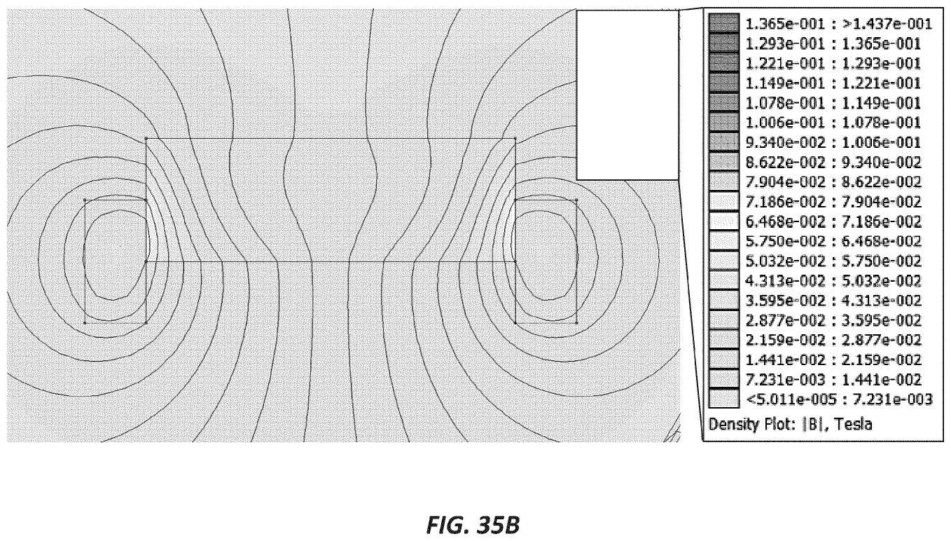

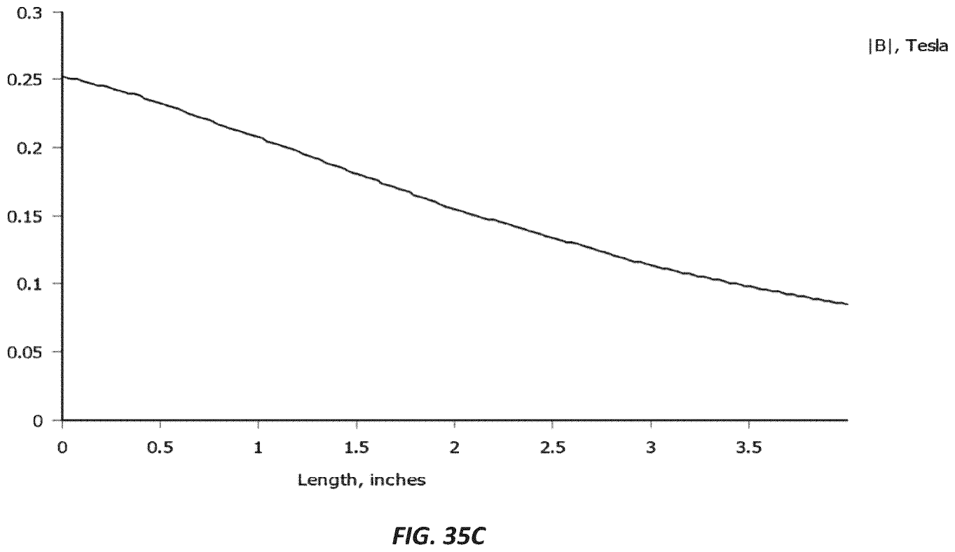

FIGS. 35A and 35B illustrate stimulations for application of a magnetic field from a helmet for clinical application in a mammalian subject (e.g., a human). A static magnetic field of 220 mT offset and an AMF (shown in FIG. 35A) may be applied. Alternatively, only an AMF may be applied (i.e., the static magnetic field may be zero) (shown in FIG. 35B). FIG. 35C is a plot of the magnetic field along the middle line of FIG. 35A.

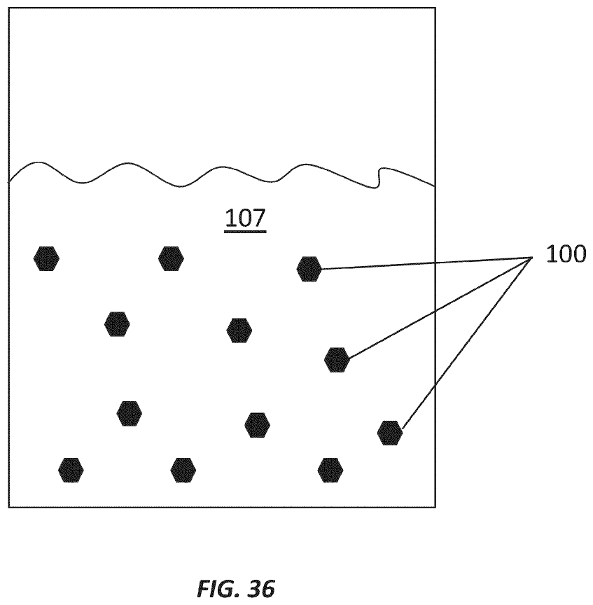

FIG. 36 illustrates a plurality of MENDs 100 dispersed in a solvent 107. The solvent 107 may be a biocompatible solvent, such as PBS. The MENDs 100 may each include an MND core, an inner shell 120, and an outer shell 130 as described above.

### Structural and Magnetic Characterization of Magnetic Nanomaterials

Structural imaging of MNDs 110, CFONDs 150, and MENDs 100 and energy-dispersive X-ray spectroscopy mapping on MEND-including neurons was performed via Scanning Electron Microscopy (SEM). Transmission electron microscopy (TEM) imaging and single-particle electron diffraction analysis was performed using a FEI Tecnai G2 Spirit TWIN TEM. The diameter and thickness of MNDs 110, CFONDs 150, and MENDs 100 were estimated from the ensemble averages of particles in TEM and SEM images. Powder X-ray diffraction patterns of as-synthesized MNDs 110, CFONDs 150, and MENDs 100 were collected by a three-circle diffractometer coupled to a Bruker-AXS Smart Apex charged-coupled device (CCD) detector with graphite-monochromated Mo K_{α} radiation (λ=0.71073 Å), and the data were processed with PANalytical HighScore Plus software. Room-temperature hysteresis curves were generated using the combined superconducting quantum interference device (SQUID) and vibrating sample magnetometer (VSM) mode of a Quantum Design MPMS-3 at 300 K. An Agilent 5100 Inductively Coupled Plasma-Optical Emission Spectrometer (ICP-OES) was used to quantify the elemental concentration for the calculation of saturation magnetization. For ICP-OES analysis, nanoparticles were digested in 37% v/v HCL overnight and diluted in 2 wt % HNO₃.

### Micromagnetic Simulations

Full magnetoelastic simulations were performed with MuMax3 magnetoelastic extension using literature values of magnetic and elastic constants for the materials, which are shown in Table 1 below, and a cell size of 2×2×2 nm³. The simulation was performed at discrete magnetic fields corresponding to the maxima of an oscillating field with an amplitude of 10 mT around an offset at 220 mT. Increasing the strength of the elastic damping constant, a steady-state elastic displacement could be achieved within a simulation time of approximately 72-168 hours running on a GeForce RTX 3060 GPU. The steady state emerged after approximately 35 ns following the initial field application, and 10 ns was required for subsequent field steps. Hence, all simulations were performed with the initial field value for 40 ns, and each subsequent field value for 15 ns.

To quantify the normalized displacement of each particle at the maxima of an oscillating field with an amplitude of 10 mT around an offset at 220 mT, the root-mean-square displacement normalized to the solid diagonal of each simulation cell was averaged. To calculate the deformation of each particle under the oscillating field with an amplitude of 10 mT around an offset at 220 mT, the difference in normalized displacements at two sequent maxima was averaged. Due to limits of MuMax3 Magnetoelastic extension the same sign in the anisotropy of the Fe₃O₄ and CoFe₂O₄ for the core shell particles was used. This may be an acceptable approximation as a lower bound of the strain in the composite particle. With a difference in anisotropy sign, the very large magnetostriction may not be simulated with the existing computational infrastructure in as the resultant pressure waves took microseconds to dissipate.

TABLE 1
Materials parameters employed in micromagnetic simulations of
magnetostriction.

| Constant | Fe₃O₄ | | CoFe₂O₄ | |
|---|---|---|---|---|
| Saturation Magnetization | 480 × 10³ | A/m | 445 × 10³ | A/m |
| Exchange Coupling Strength | 1.3 × 10⁻¹² | J/m | 1.7 × 10⁻¹¹ | J/m |
| Interlayer exchange coupling | 1 × 10⁻¹² J/m to Co | | 1 × 10⁻¹² J/m to Fe | |
| Crystalline Anisotropy Axis | (1, 1, 1) | | (1, 1, 1) | |
| Crystalline Anisotropy constant | 13500 | J/m³ | 240000 | J/m³ |
| Density | 5110 | kg/m³ | 5230 | kg/m³ |
| Magnetoelastic coupling constant | 1.6 × 10⁷ | J/m³ | 3.2 × 10⁷ | J/m³ |

| | | |
|---|---|---|
| C11, C12, C44 | 240 × 10⁹, 135 × 10⁹, | 244 × 10⁹, 142 × 10⁹, |
| | 87 × 10⁹ N/m² | 75 × 10⁹ N/m² |

### Design and Fabrication of Electromagnets

For magnetoelectric coupling coefficient measurements and Ca imaging in vitro, TEMCo 14 AWG copper magnet wire was wound around a C-shaped magnetic core with an 8 mm gap. The coil was connected to a power supply (e.g., a Crown DC-300A Series II) and signal generator (e.g., a Picoscope 2204A) to generate a magnetic field combining a static magnetic field offset (OMF, magnitude: 0-320 mT) and an alternating magnetic field (AMF, frequency: 0-1000 Hz, amplitude: 0-14 mT). For in-vivo experiments, separate apparatuses generated the OMF and AMF to generate a uniform, time varying, magnetic field over a larger volume. To generate the AMF (10 mT, 150 Hz) for c-Fos expression and fiber photometry experiments, a TEMCo 14 AWG copper magnet wire solenoid with a 10 cm inner diameter was connected to the power supply and the signal generator. To generate the DC magnetic field (220 mT), a ring-shaped permanent magnet (3.81 cm od×1.905 cm id×0.3175 cm thick, K&J Magnetics, Inc. RX8C2) was used and it was positioned in the center of the AMF coil.

FIGS. 4J and 31 show custom arena or behavior apparatus 480 or 3180 for applying magnetic fields during behavior experiments. The custom arena or behavior apparatus 480 or 3180 may be comprised of two-connected cylindrical chambers (e.g., a FixtureDisplays® clear acrylic tube, 75 mm diameter, 2 mm wall thickness, and 205 mm long) that was divided into two stimulation chambers 481, 482, 3181, and/or 3182 (11 cm) separated by a neutral chamber (19 cm) that contained the animal entry port 3108. The entry port 3108 may be covered with 3D printed acrylic curved plate after the entrance of mouse. To generate OMF during behavioral experiments, two rectangular permanent magnets 485 or 3185 (7.62 cm×7.62 cm×1.27 cm thick, K&J Magnetics, Inc. BZ0Z08-N52) were placed at the top and bottom of each stimulation chamber 481, 482, 3181, and/or 3182. To generate AMFs during behavioral experiments, solenoids 484 or 3184 (e.g., a TEMCo 14 AWG copper magnet wire) were wound around each stimulation chamber 481, 482, 3181, and/or 3182 and connected to the power supply and the signal generator.

### Magnetoelectric Coupling Coefficient Measurements

A three-electrode electrochemical cell was used to measure the potential used to maintain the surface charge, which fluctuated according to the electric polarization of the MENDs in the presence of a magnetic field. To fit within the 8 mm gap of the C-shaped electromagnet described above, a nuclear magnetic resonance (NMR) tube (8 mm outer diameter 0.5 mm thickness) was used as the electrochemical cell. Within the cell, the Ag/AgCl reference electrode, Pt counter electrode, and working electrode were immersed in Tyrode's solution. The working electrode was prepared by drop-casting MEND solution onto a 0.3×1.2 cm² indium tin oxide (ITO) glass substrate and connecting it to a copper wire using conductive silver epoxy (e.g., MG Chemicals 842AR-15ML). The electrical connection was then sealed with epoxy (H.B. Fuller, 10010217) for insulation. The thickness of the MEND film on the ITO substrate was measured via a profilometer (e.g., a Bruker Dektak DXT-A Stylus). All electrochemical measurements were performed using a potentiostat (e.g., a Gamry Interface 1010E).

### Fluorescent Imaging in Cultured Hippocampal Neurons

All animal procedures were approved by the Massachusetts Institute of Technology Committee on Animal Care (MIT CAC). Hippocampal neurons were extracted from neonatal rat (Sprague Dawley, 001) pups (P1) and dissociated with Papain (Worthington Biochemical). The cells were then seeded on glass slides (5 mm diameter, Bellco Glass 1943-00005) in 24 well plates at a density of 112500 cells/mL. Prior to seeding, the glass slides were cleaned by evaporating ethanol with an alcohol lamp and then coated with Matrigel® (Corning). The cells were maintained in 1 mL Neurobasal media (Invitrogen). Glial inhibition was conducted with 5-fluoro-2′-deoxyuridine (FUDR, F0503 Sigma) 3 days after seeding. Four days following seeding, the neurons were transduced with 1 μL of an adeno-associated virus serotype 9 (AAV9) carrying a fluorescent calcium ion indicator GCaMP6s under a pan-neuronal human synapsin (hSyn) promoter (AAV9-hSyn::GCaMP6s, Addgene viral prep #100843-AAV9, >1×10¹³ IU/μL). After 5 days of incubation, calcium (Ca²⁺) imaging with GCaMP6s experiments were performed.

For Ca²⁺ imaging, the hippocampal neurons were washed once with Tyrode's solution and then immersed into 0.1 mg/mL MEND solution within a well of 24-well plate for 1 hour to obtain ~1 g_{MEND}/mm² density of the particles on the neuronal surfaces. To change the MEND density on neuronal surfaces, the concentration and volume of the MEND incubation solution were varied. The hippocampal neurons on the glass coverslips were then transferred into a custom sample holder containing 200 μL Tyrode's solution, which was introduced into an electromagnet described above. The fluorescence changes in GCaMP6s were recorded at a rate of 1 frame per second (fps). The fluorescence intensity of each cell was analyzed with ImageJ software, and F₀ values were determined as the average fluorescence intensity during 30 seconds prior to the initial application of the AMF. Averaged ΔF/F₀ was estimated from 300 randomly selected neurons from 3 plates. The number of the peaks in ΔF/F₀ was counted when the peak height is larger than the half of mean standard deviation of ΔF/F₀ for the overall imaging time of each video. The cells were defined as responsive if ΔF/F₀ value exceeded three times the standard deviation (3σ) of the baseline (collected over 30 s prior to the initial application of the AMF) within 15 seconds from MF onset.

To examine the cell viability in the presence of MENDs following MF stimuli, a Live/Dead Viability/Cytotoxicity Kit (e.g., Invitrogen, L3224) analysis was performed. The live and dead cells were indicated with Calcein AM, green and ethidium homodimer-1, red. The proportion of viable cells calculated by normalizing the viable cell number to the total number of cell nuclei based on Hoechst staining (Thermo Scientific, Hoechst 33342 Solution (20 mM)).

The neurons were incubated with the assay reagents for 20 minutes in 37° C. and then imaged before and after three cycles of MF field via a fluorescent microscope (e.g., an Olympus IX73, 20× objective lens).

### Stereotactic Surgeries

All animal procedures were approved by the MIT CAC. Surgeries were conducted under aseptic conditions within a stereotaxic frame (David Kopf Instruments). Mice were anesthetized under isoflurane (0.5-2.5% in O₂) using an anesthesia machine (VET EQUIP). During the surgery, the eyes were covered with ophthalmic ointment, and a heat pad was used to maintain the animals' core temperature. Mice were provided with subcutaneous injections of 0.6 mL of sterile Ringer's solution for hydration and extended-release buprenorphine (1 mg kg⁻¹) for analgesia at the start of the procedure. The head was fixed into position using ear bars, then the fur on the top of the head was removed using depilatory cream. Following sterilization of the skin with betadine and ethanol, a midline incision was made along the scalp. Coordinates for the injection/implantation site were established based on the Mouse Brain Atlas. A small craniotomy was drilled through the skull using a rotary tool (e.g., a Dremel Micro 8050) and a carbon steel burr (e.g., a Heisinger, 19007-05), and the dura was gently removed. Particles were injected into the VTA (anterior-posterior, AP −2.9 mm, medial-lateral, ML −0.5 mm, dorsal-ventral, DV −4.5 mm) using a microinjection apparatus (10 μL Hamilton syringe #80308, UMP-3 syringe pump, and Micro4 controller; all from World Precision Instruments).

For c-Fos expression analysis and behavioral assays, 1.5 μL of MEND 100 particles, MND 110 control particles, or PBS were injected at a rate of 500 nl min⁻¹. For MENDs 100 and MND 110 nanoparticle solutions, a concentration of 1.5 mg mL⁻¹ was used for all experiments, except for an additional 0.5 mg mL⁻¹ low concentration of MENDs 100 tested in the c-Fos expression experiments. For PBS controls, the same volume of sterile phosphate buffered saline was injected instead of particles. After injection, the syringe was lifted up by 0.1 mm from the initial DV coordinate and left in place for 10 minutes before slowly withdrawing (0.5 mm min⁻¹), and then the skin was sutured (Ethicon, Ethilon 661H, polyamide 6, 19 mm needle length).

For the mice used for fiber photometry experiments, 300 nL of AAV9 hSyn::GCaMP6s, Addgene viral prep #100843-AAV9, >1×10¹³ IU/μL) was injected along with the 1.3 μL particles or PBS. The AAV solution was loaded into the syringe tip after the particles or control PBS, such that it was injected first and immediately followed by the particle or PBS injection into the brain. Following the injection, the syringes were lifted as described above. At 0.1 mm above the injection coordinates, a 200 μm-diameter silica fiber (Thorlabs FT200EMT) with a 2.5 mm diameter stainless steel ferrule (Thorlabs SF230-10) was implanted and cemented in place with adhesive acrylic (C&B-Metabond, Parkell) and followed with dental cement (Jet Set-4).

Following surgery, a subcutaneous injection of carprofen (5 mg kg⁻¹) was provided as an anti-inflammatory and analgesia agent, and the animals were placed in a clean recovery cage, part of which was positioned on a heating pad with ad libitum access to water and diet gel wet food.

### Fiber Photometry

Fiber photometry recordings were performed using a Neurophotometrics fiber photometry system (FP3002, Neurophotometrics LTD). The fiber photometry system 2890 is illustrated in FIG. 28A. This system utilized a blue (peak wavelength k=470 nm) light-emitting device (LED) 2895 to excite GCaMP6s and a violet (peak k=415 nm) LED as an isosbestic wavelength control, with a fluorescence light path that included a dichroic mirror 2896 to pass emitted green fluorescence (passband 495-530 nm) onto a complementary metal-oxide semiconductor camera 2897 (FLIR BlackFly). The 470 nm and 415 nm LEDs were each calibrated to provide 75 μW of optical power out of the tip of a 200 μm silica fiber 2809 matching the type implanted into the animals (Thorlabs FT200EMT). The system was coupled to a low-autofluorescence branching bundle patch cord (400 μm core, 0.57 NA, Doric), which was connected to the animal's implant using a ceramic split mating sleeve (Thorlabs ADAF1). All photometry recordings were performed under isoflurane anesthesia (0.5-2.5% in O₂, VET EQUIP) using a nose cone. After induction, the patch cord was connected to the animal's fiber implant, and the animal's head was placed in the center of the custom magnetic apparatus described above: 10 cm diameter solenoid AMF coil with a ring-shaped static magnet inside to provide OMF, whose centers are aligned.

Fiber photometry recordings were performed at 130 Hz sampling rate. After recording a 5-minute baseline to account for rapid photobleaching at the start of the experiment, 2-second pulses of magnetic field (OMF 220 mT, AMF 150 Hz, 10 mT) were applied every 180 seconds. Each animal received 10 total pulses of magnetic stimulation. Data were collected in Bonsai software and exported to MATLAB (MathworksR2022a) for analysis. Photometry data were analyzed as follows: data were low-pass filtered below 25 Hz (2^{nd} order Butterworth filter), both isosbestic (405 nm excitation wavelength) and Ca²⁺-sensitive (470 nm excitation wavelength) signals were fitted with MATLAB exp2 fitting function, and then the fitting functions were subtracted from the original signals to correct for photobleaching. To remove the motion artifacts, the baseline corrected isosbestic signal was subtracted from the baseline-corrected 470 nm signal. The fluorescence average of 30 seconds prior to the first stimulation epoch was taken as F₀, and ΔF/F₀ was segmented corresponding to each stimulation epoch (30 second pre-stimulation and 150 seconds post-stimulation).

### Immunohistochemical Quantification of Biomarkers

For the c-Fos expression experiments, mice were anesthetized one week after the surgery via an intraperitoneal injection of ketamine (100 mg/kg) and xylazine (10 mg/kg) mixture. The heads of the anesthetized mice were placed into the center of a custom-built apparatus with a 10 cm diameter solenoid AC coil outside and ring-shaped static magnet inside, whose centers are aligned. Each mouse received three 2-second pulses of magnetic stimulation (220 mT OMF; 150 Hz, 10 mT AMF) separated by 90-minute rest epochs prior to sacrificing. Following the 90-minute c-fos induction period, the anesthetized mice were euthanized by a lethal intraperitoneal injection of a sodium pentobarbital (Fatal-plus, 50 mg mL⁻¹, dose 100 mg/kg). The mice were then transcardially perfused with PBS and 4% paraformaldehyde (PFA), and their brains were extracted and kept in 4% PFA overnight at 4° C. After moving the fixed tissue to PBS and storing at 4° C. for 24 hours, the brains were sectioned into 50 μm coronal slices with vibrating blade microtome (Leica VT1000S). The permeabilization was done on the slices for 30 minutes in the dark at room temperature in a 0.3% v/v Triton X-100 solution in PBS, and then the blocking was done for 1 hour with 0.3% v/v Triton X-100 and normal donkey serum 5% v/v blocking serum solution in PBS with an orbital shaker. Following PBS washing three times, the brain slices were incubated in the first antibody solution overnight at 4° C. After three washes with PBS, the brain slices were immersed in a secondary antibody solution for 2 hours at room temperature on the orbital shaker in a dark room.

After another three washes with PBS, the brain slices were stained with DAPI, washed, and then transferred onto glass slides with mounting medium (Fluoromount G, Southern Biotech). Rabbit anti-cfos (1:500, Cell Signaling Technology, 2250 second) primary antibodies and Donkey anti-rabbit Alexa Fluor 488 (1:1000, Invitrogen, A-21206) secondary antibodies were used for the c-fos expression analysis. Three pairs of primary and secondary antibodies were used for the toxicity assessment and summarized in Table 2 below.

TABLE 2
Antibodies used for biocompatibility analysis.

| | Primary/Secondary | |
|---|---|---|
| Antibody | (Dilution) | Product Information |
| Goat anti-Iba1 antibody | Primary Antibody (1:500) | Abcam ab107159 |
| Goat anti-GFAP antibody | Primary Antibody (1:1000) | Abcam ab53554 |
| Rabbit anti-CD68 antibody | Primary Antibody (1:250) | Abcam ab125212 |
| Donkey anti Rabbit IgG | Secondary Antibody (1:1000) | Thermo Scientific A-21206 |
| (H + L), Alexa Fluor 488 | | |
| Donkey anti Goat IgG (H + L), | Secondary Antibody (1:1000) | Fisher Scientific A-21082 |
| Alexa Fluor 633 | | |

### Behavioral Assays

For behavioral experiments, the mice were tested during the light phase of the 12-hour light/dark cycle. For the place preference assay, 71 mm inner diameter acrylic cylinder was divided into three chambers—two 11 cm long stimulation chambers (solid 482 or 3182 and stripe-patterned 481 or 3181 bottom) separated by a 19 cm long neutral chamber 483 or 3183 (purple colored bottom). The stimulation chambers 481, 482, 3181, and 3182 were equipped with custom-made electromagnets (e.g., AMF coil 484 or 3184) and static magnets (e.g., 485 or 3185) outside the stimulation chambers 481, 482, 3181, and 3182, as shown in FIGS. 4J and 31. For three days (Day −2 to Day 0, FIGS. 4J and 31) before the baseline place preference test, the mice were habituated daily for 15 minutes in the setup and to the researchers. On Day 1, the mice were placed into the arena 480 or 3180 for 5-minute habituation during which they could explore the chambers 481, 482, 483, 3181, 3182, 3183 freely without any stimulation, and their place preference was tested for the following 10 minutes without any magnetic stimulation. From Day 2 to Day 4, following a 5-minute habituation in the setup, the mice were stimulated with the magnetic field when they entered the less-preferred chamber for 10 minutes daily. On Day 5, the place preference was tested again without any stimulation. The mouse location in the arena 480 or 3180 was recorded with two cameras (e.g., a Logitech HD Pro Webcam C920) positioned at the ends of each stimulation chamber 481, 482, 3181, or 3182 and Logitech Capture Bio Recording & Streaming Software (2.08.11) was used to record the videos. Analysis of the place preference assay was done manually, where the observer was blinded to the subject type and marked the time spent in each chamber. Mice that showed more than 500 seconds (total test time is 600 seconds) preference to a chamber during pre-test or stayed 0 seconds at the stimulated chamber on Day 2 were eliminated from the subsequent analyses.

### Statistical Analyses

OriginPro 2019 was used for assessing the statistical significance of all comparisons in this study. Although sample sizes were not determined with the power analysis, the group sizes for immunohistochemistry and behavior tests were decided to be similar with previous research performed in the same brain circuit. For c-Fos expression analysis, the data distribution was tested for normality, and then analyzed with ANOVA followed by Tukey's post-hoc comparison test (*P<0.05, **P<0.01, ***P<0.001). For the immunohistochemistry analyses relevant to the toxicity assessment, unpaired t-test was used to assess the differences between two groups, where significance threshold was indicated with n.s., P>0.05, *P<0.05, **P<0.01, ***P<0.001. For behavioral experiments, a Kruskal Wallis followed by Tukey's post-hoc comparison test was applied to compare three groups simultaneously with thresholds of n.s. P>0.05, *P<0.05, **P<0.01, ***P<0.001, ****P<0.0001. For the comparison of Pre- and Post-learning days within each group, paired t-test was performed when the data distribution was found normal. For non- normal distribution, Wilcoxon Signed Racks Test was performed. For the preference change data comparing all three groups, the data followed normality, and thus one-way ANOVA was used followed by Tukey's post-hoc comparison test.

### Magnetoelectric Coefficient

The magnetoelectric coefficient, α_{ME}, of the MENDs 100 was measured to be 150 mV mT⁻¹ cm⁻¹ at the combined magnetic field (MF) conditions: offset magnetic field (OMF) 220 mT and alternating magnetic field (AMF) with a frequency f_{AC}=150 Hz and amplitude 10 mT (FIGS. 1H-1J). Using the particle diameter of 231 nm and the AMF magnitude of 10 mT, the potential generated by an individual MEND 100 at these MF conditions may be calculated as approximately

$$35 μ V ( \frac{150 mV}{mT \cdot cm} \times 200 nm \times 10 mT = 34.7 μ V ) ,$$

which is significantly below the threshold for neuronal activation and action potential firing (15-30 mV). Without being bound by any particular theory or hypothesis the mechanism underlying the MEND-mediated neuronal modulation a model was developed based on: (1) The instantaneous change in membrane potential, ΔV, for each pulse of the AMF resulting from the integration of voltages generated by individual MENDs 100 distributed with a spacing d on the cell membrane and (2) the dynamic change in membrane potential V(t) resulting from the temporal summation of ΔV across subsequent periods of the AMF.

To model the system, solutions to the three-dimensional cable equation were leveraged, where neuron processes are approximated as cylindrical bodies, with radius a₂. This framework may enable the calculation of the change in membrane potential in the presence of multiple point sources of current or voltage (e.g., microelectrodes or MENDs 100) distributed across the membrane surface. As the classic cable model is limited to two microelectrodes, it was expanded it to include a larger number of sources for the membrane potential fluctuations. This allowed the change in membrane potential ΔV as a function of the distance between MENDs 100, d, and the potential generated by a single MEND 100, V₀, to be calculated.

A neuron receiving stimulation via MENDs 100 on its membrane as a cylindrical cell receiving current injection from a series of microelectrodes with spacing d and zero circumferential angle (θ=0) aligned along the cell surface was modeled (see FIGS. 20A-20D). For two microelectrodes separated by the distance d that supply current i₀, the change in membrane potential, ΔV, according to the steady state solution of the cable model is given by Equation 1:

$$\frac{150 mV}{mT \cdot cm} \times 200 nm \times 10 mT = 34.7 μ V \qquad Equation 1$$

where rᵢ is the resistance of a unit length of the interior of the cell against longitudinal current flow, a is the radius of the cell, and L and S represent the spatial decay of potential caused, respectively, by the one-dimensional and three-dimensional spread of current L(d) is defined using Equation 2:

$$L ( d ) = \frac{λ}{a} {e}^{- d / λ} , \qquad Equation 2$$

where λ is the neuron length constant, defined as the length over which the membrane potential decays to V₀/e. Values of S(d) have previously been empirically determined, and were tabulated for varying values of d, shown in Table 1.

MENDs 100 on the neuron membrane can be represented as multiple microelectrodes spaced at a distance d, which we assume to be much smaller than the overall length of the neuron, l. Thus, the change in membrane potential during one stimulation pulse integrated across all microelectrodes is given using Equation 3:

$$Δ V ( d , {i}_{0} ) = \vert {\int }_{d}^{l} \frac{d V}{d x} dx \vert = \vert V ( l ) - V ( d ) \vert = \vert 0.5 {r}_{i} {i}_{0} a ( L ( l ) - L ( d ) + S ( l ) - S ( d ) ) \vert . \qquad Equation 3$$

In the approximation that the potential change at a large distance from the point source approaches zero, we can assume that L(l)≈0 and S(l)≈0, which leads to a simplification of Equation 3 to Equation 4:

$$Δ V ( d , {i}_{0} ) \approx \vert 0.5 {r}_{i} {i}_{0} a ( - L ( d ) - S ( d ) ) \vert . \qquad Equation 4$$

Given that MEND-mediated modulation effectively relies on voltage application rather than current injection, we applied a steady-state solution from the cable model to calculate current io from the MEND 100 potential V₀⁴ given using Equation 5:

$${i}_{o} = \frac{2 {V}_{0}}{{r}_{i} λ} , \qquad Equation 5$$

where λ is the neuron length constant (defined above) determined by rᵢ. and the membrane resistance per unit length. Hence, the integrated membrane potential change for a single pulse of the AMF can be rewritten in terms of d and V₀ using Equation 6:

$$Δ V ( d , {V}_{0} ) = \vert 0.5 {r}_{i} {i}_{0} a ( - L ( d ) - S ( d ) ) \vert = \vert 0.5 {r}_{i} \frac{2 {V}_{0}}{{r}_{i} λ} a [ - \frac{λ}{a} {e}^{- \frac{d}{λ}} - S ( d ) ] \vert . \qquad Equation 6$$

The values for a, λ, r, and S(d), taken from the literature, are tabulated in Table 2. This equation implies that the total change in membrane potential ΔV(d, V_{0′}) may increase with decreasing spacing between the individual particles, d, and with increasing voltage generated from individual particles, V₀.

Using these insights about membrane potential change stemming from the integrated inputs from spatially distributed MENDs 100, a framework to determine how the membrane potential varies in time across multiple cycles of AMF was developed. Application of AMF to a neuron decorated with MENDs 100 is anticipated to yield temporal summation of the subthreshold potentials generated by the MENDs 100 during each half-period of the AMF. (Note that since the ME effect does not depend on the AMF sign, the frequency of voltage fluctuations in MENDs 100 may be 2×f_{AC}, where f_{AC} is the AMF frequency.) This temporal summation of a series of subthreshold potentials is known as neuronal facilitation. To simulate neural facilitation during magnetoelectric stimulation with MENDs 100, a stochastic model of the repetitive activity of neurons was adopted and combined with the three-dimensional cable model of the voltage distribution derived above in Equation 6.

When a neuron is subjected to a series of potential pulses with a given time interval between them, the membrane potential, V(t), is the sum of two components: N(t), a noise term that accounts for stochastic fluctuations in membrane potential, and D(t), a function describing the reestablishment of resting membrane potential following each applied potential pulse. Since N(t) is a noise term with a mean of 0 mV, on average, V(t) reaches the threshold for neuron spiking when D(t) does so. D(t) is dependent on the state of the membrane potential at the onset of the applied potential pulse, and it is defined using Equation 7:

$$D ( t ) = {D}_{F} + ( {D}_{I} - {D}_{F} ) {e}^{\frac{- ( t - R )}{k}} , \qquad Equation 7$$

where D₁ is the membrane potential at the onset of the applied potential, D_{F} is the membrane potential in the fully-recovered state dependent on the level of excitatory input, R is the absolute refractory period of the neuron spiking (R=0.7 s was used), and k is the time constant of decay of the after-potential (k=9 ms was fixed in this simulation). If the sub-threshold voltage delivered by MENDs 100 is considered to be akin to excitatory input to the neuron, then D_{F} may depend on ΔV(d, V₀), as defined in Equation 6. D₁ varies depending on history, and it is defined as one-half of the difference between −90 mV and the value of D(t) at the onset of the applied potential. Thus, for each period of the applied

$$AMF , {D}_{F}^{′} = {D}_{F , initial} + Δ V and {D}_{I}^{′} = ( {D}_{F}^{′} - 90 mV ) / 2$$

with these values getting updated for every cycle of the AMF.

According to this model, subthreshold potentials of the MENDs 100 give rise to a potential change ΔV(d, V₀) for each period of the AMF, which decays according to D(t) (Equation 7). With an applied AMF of 150 Hz, subsequent voltage impulses delivered by the MENDs 100 summate over time until the critical threshold is crossed and the neuron fires.

Using this model, it was found that neurons with a resting membrane potential of −70 mV decorated with MENDs 100 and exposed to 150 Hz AMF may be depolarized to the critical threshold for action potential firing, −55 mV, after ≥2 seconds, for a single MEND 100 potential V₀>24 μV when inter-MEND spacing d is 0.25a (FIGS. 3E-3G).

TABLE 3
S(d) at different values of MEND spacing
d in fractions of the axonal radius a.³

| d | S(d) |
|---|---|
| 0.25a | 3.202 |
| 0.5a | 1.212 |
| 0.75a | 0.598 |
| 1a | 0.327 |
| 2a | 0.042 |

TABLE 4
Parameters used to model MEND-mediated
neuronal excitation with AMF.

| Parameter | Value | |
|---|---|---|
| Axial resistivity, Rᵢ | 150 | Ωcm |
| Axial resistance, rᵢ | Rᵢ/πa² | |
| Axonal radius, a | 2 | μm |
| Length constant, λ | 258 | μm |

TABLE 5
Coercivity (H_{C}) and saturation magnetization
(Ms) of MND 110, CFOND 150, and MEND 100.

| | H_{C} (mT) | Ms (emu/g) |
|---|---|---|
| MND 110 | 38.97 ± 0.76 | 123.34 ± 8.68 |
| CFOND 150 | 37.27 ± 4.70 | 113.42 ± 8.97 |
| MEND 100 | 66.00 ± 7.37 | 116.64 ± 4.78 |

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of making an anisotropic magnetoelectric nanoparticle, the method comprising:
forming a magnetite nanodisc via reduction of hexagonal nanodisc hematite;
forming a magnetostrictive shell on the magnetite nanodisc; and
forming a piezoelectric shell on the magnetostrictive shell to yield the anisotropic magnetoelectric nanoparticle.

2. The method of claim 1, wherein the magnetite nanodisc has a maximum lateral dimension of about 170 nm to about 550 nm and a maximum thickness of up to about 50 nm.

3. The method of claim 1, wherein forming the magnetite nanodisc via reduction of hexagonal nanodisc hematite comprises controlling a percentage of water in the reduction.

4. The method of claim 3, wherein the percentage of water is 6-9%.

5. The method of claim 1, wherein forming the magnetostrictive shell on the magnetite nanodisc comprises growing a plurality of layers of magnetostrictive material on the magnetite nanodisc.

6. The method of claim 5, wherein the magnetostrictive shell comprises AₓFe₃₋ₓO₄, where A is one of Co, Mn, Ni, Fe, Zn, Mg, or Cu.

7. The method of claim 1, wherein forming the magnetostrictive shell on the magnetite nanodisc comprises growing magnetostrictive material to a thickness of up to about 5 nm on the magnetite nanodisc.

8. The method of claim 1, wherein forming the piezoelectric shell on the magnetostrictive shell comprises depositing piezoelectric material on the magnetostrictive shell to a thickness of up to about 5 nm.

9. The method of claim 8, wherein the piezoelectric shell comprises one of barium titanate (BaTiO₃), lead zirconate titanate (PZT) (Pb[ZrₓTi₁₋ₓ]O₃ (0≤x≤1)), lead titanate (PbTiO₃), or barium-calcium zirconium titanate (BCZT).

10. The method of claim 1, wherein the anisotropic magnetoelectric nanoparticle is one of a plurality of anisotropic magnetoelectric nanoparticles, and further comprising:
dispersing the plurality of anisotropic magnetoelectric nanoparticles in a biocompatible solvent.

11. An anisotropic magnetoelectric nanoparticle comprising:
a magnetite core having a maximum lateral dimension of about 170 nm to about 550 nm and a maximum thickness of up to 50 nm;
a magnetostrictive shell on the magnetite core; and
a piezoelectric shell on the magnetostrictive shell.

12. The anisotropic magnetoelectric nanoparticle of claim 11, wherein the magnetostrictive shell has a thickness of about 5 nm and the piezoelectric shell has a thickness of about 5 nm.

13. The anisotropic magnetoelectric nanoparticle of claim 11, wherein the magnetostrictive shell comprises an AₓFe₃₋ₓO₄, where A is one of Co, Mn, Ni, Fe, Zn, Mg, or Cu.

14. The anisotropic magnetoelectric nanoparticle of claim 13, wherein A is Co.

15. The anisotropic magnetoelectric nanoparticle of claim 11, wherein the piezoelectric shell comprises one of barium titanate (BaTiO₃), lead zirconate titanate (PZT) (Pb[ZrₓTi₁₋ₓ]O₃ (0≤x≤1)), lead titanate (PbTiO₃), or barium-calcium zirconium titanate (BCZT).

16. The anisotropic magnetoelectric nanoparticle of claim 15, wherein the piezoelectric shell is barium titanate (BaTiO₃).

17. The anisotropic magnetoelectric nanoparticle of claim 11, wherein the anisotropic magnetoelectric nanoparticle has a magnetoelectric coupling coefficient of about 130-180 mV/mT-cm under a 220 mT, 150 Hz alternating current magnetic field.

18. The anisotropic magnetoelectric nanoparticle of claim 11, wherein the magnetite core is hexagonal.

19. An anisotropic magnetoelectric nanoparticle comprising:
an anisotropic magnetite core;
a layer of AₓFe₃₋ₓO₄ disposed on the anisotropic magnetite core, where A is one of Co, Mn, Ni, Fe, Zn, Mg, or Cu; and
a layer of piezoelectric material disposed on the layer of AₓFe₃₋ₓO₄.

20. The anisotropic magnetoelectric nanoparticle of claim 19, wherein the anisotropic magnetoelectric nanoparticle has a magnetoelectric coupling coefficient of about 130-180 mV/mT-cm under a 220 mT, 150 Hz alternating current magnetic field.
